(19) United States
(12) Patent Application Publication
(10) Pub. No.: US 2016/0326540 A1
(43) Pub. Date: Nov. 10, 2016

(54) Methods and Compositions Employing a Sulfonylurea-Dependent Stabilization Domain

(71) Applicant: PIONEER HI-BRED INTERNATIONAL, INC., Johnston, IA (US)

(72) Inventor: Kevin E. McBride, Davis, CA (US)

(21) Appl. No.: 14/775,575

(22) PCT Filed: Mar. 11, 2014
(86) PCT No.: PCT/US2014/023573

§ 371 Date: Sep. 11, 2015

Related U.S. Application Data
(60) Provisional application No. 61/776,124, filed on Mar. 11, 2013.

(51) Int. Cl. C12N 15/82; C07K 2/00; C12P 21/00

(52) U.S. Cl. CPC C12N 15/8257; C12P 21/00; C07K 2/00; C07K 2319/31; C07K 2319/92

(57) ABSTRACT

Methods and compositions are provided which employ polypeptides having a SU-dependent stabilization domain, and nucleotide sequences encoding the same. Such SU-dependent stabilization domains can be employed a part of a fusion protein comprising a polypeptide of interest. The presence of the SU-dependent stabilization domain in such a fusion protein serves as a method of modulating the level of the protein of interest through the presence of or the absence of a SU ligand. Further provided are methods and compositions employing the SU-dependent stabilization domain in a SuR or revSuR. Such polypeptides can be employed in combination with a chemical-gene switch system to allow for a sophisticated level of transcriptional control.

### CROSS-REFERENCE TO RELATED PATENT APPLICATIONS

This present application is a National Phase Under 35 U.S.C. §371 of PCT/US2014/023573 filed in the Patent Cooperation Treaty U.S. Receiving Office on Mar. 11, 2014, which claims the priority of and the benefit of the filing dated of U.S. Provisional Patent Application Ser. No. 61/776,124, filed Mar. 11, 2013, the entire contents of which are herein incorporated by reference.

### REFERENCE TO SEQUENCE LISTING SUBMITTED ELECTRONICALLY

The Sequence Listing submitted Jul. 13, 2016, as a text file named 36446_0070U2_July_updated_Sequence_Listing.txt, created on Jul. 11, 2016, and having a size of 2,375,358 bytes is hereby incorporated by reference pursuant to 37 C.F.R. §1.52 (e)(5).

### FIELD OF THE INVENTION

The invention relates to the field of molecular biology, more particularly to the regulation of gene expression.

### BACKGROUND

Chemical based control of transcription in plants with sulfonylurea (SU) herbicides via a modified tet-repressor based mechanism has been demonstrated (US20110294216). This strategy relies on repression/de-repression of fully functional promoters having embedded tet operator sequences thru co-expression of conditional repressor proteins (Gatz et al. (1988) PNAS 85:1394-1397; Frohberg et al. (1991) PNAS 88:10470-10474; Gatz et al. (1992) The Plant Journal 2:397-404; Yao et al. (1998) Human Gene Therapy 9:1939-1950), yet could be modified to create a SU controlled transcriptional activator acting on a minimal promoter with upstream tet operators (Gossen et al. (1995) Science 268:1766-1769).

Alternative methods of SU dependent regulation are needed to produce systems that can, if desired, reduce genetic complexity to one expression cassette instead of two (transcriptional regulation requires one cassette for the target gene and one cassette for the transcriptional activator/repressor) and possibly enable a quicker response to ligand. One method to accomplish this is to regulate the stability of any protein of interest by fusion to chemically responsive stability tags (A general chemical method to regulate protein stability in the mammalian central nervous system. Iwamoto, M. et al. (2010) Chemistry and Biology 17:981-988; also see ‘ProteoTunef’—Clontech). Such methods and compositions can find use either alone or in combination with other gene-chemical switch systems to enhance regulation of gene expression.

### SUMMARY

Methods and compositions are provided which employ polypeptides having a SU-dependent stabilization domain, and nucleotide sequences encoding the same. Such SU stabilization domains can be employed as part of a fusion protein comprising a polypeptide of interest. The presence of the SU-dependent stabilization domain in such a fusion protein serves as a method of modulating the level of the protein of interest through the presence of or the absence of a SU ligand.

Further provided are methods and compositions employing the SU-dependent stabilization domain in a SU chemically-regulated transcriptional activator, such as, SuR or a SU chemically-regulated reverse transcriptional repressor (revSuR) fused to a transcriptional activation domain. Such polypeptides can be employed in combination with a chemical-gene switch system to allow for a sophisticated level of transcriptional control.

### BRIEF DESCRIPTION OF DRAWINGS

FIG. 1 provides a schematic illustrating how ligand binding rescues stability of the fusion protein comprising the SU-dependent stabilization domain and the polypeptide of interest.

FIG. 2 provides a schematic for testing conditional stability of wild type and mutant TetR::GFP fusion proteins in Saccharomyces cereviseae.

FIG. 3 graphically shows that destabilization mutations in TetR have a greater effect on differential stability+/−anhydrotetracycline.

FIG. 4 provides a schematic of the constructs that compare Tet and SU repressors for ligand gated stability in Saccharomyces cereviseae.

FIG. 5 provides quantitative GFP fluorescence+/−sulfonylurea or anhydrotetracycline ligands in Saccharomyces cereviseae.

FIG. 6 provides the ratio of GFP::Repressor fusion protein accumulation in the presence vs. absence of anhydrotetracycline or sulfonylurea treatment in Saccharomyces cereviseae.

FIG. 7 provides anhydrotetracycline and sulfonylurea dose response data in Saccharomyces cereviseae.

FIG. 8 provides demonstration of constitutive behavior of repressors with DNA binding domain mutation L17G in E. coli B-galactosidase assays.

FIG. 9 provides a demonstration of ligand dependent EsR^{L17G}::GFP accumulation in transgenic tobacco. The construct pHD2033-2036 is set forth in SEQ ID NO: 2111. Within SEQ ID NO: 2111, the promoter comprising 35S::3×Op is between nucleotides 177 to 623, the ESR (L19G) coding region is between nucleotides 699 to 1319, the coding region for GFP is between nucleotides 1326 to 2039, the coding region of HRA is between nucleotides 4738 to 6708, and the SAMS promoter is between nucleotides 3428-4737.

FIG. 10 provides a demonstration of compatibility between the protein stability and transcriptional switch mechanisms.

The construct pHD2037-2040 is set forth in SEQ ID NO: 2112. Within SEQ ID NO: 2112, the promoter comprising 35S::3×Op is between nucleotides 177 to 623, the ESR (L19G) coding region is between nucleotides 699 to 1319, the coding region for GFP is between nucleotides 1326 to 2039, the promoter comprising g35S::3×Op is between nucleotides 3253-3699, the coding region of ESR(L13) is between nucleotides 3775 to 4395, the SAMS promoter is between nucleotides 5462 to 6771 and the HRA coding region is between nucleotides 6772 to 8742.

FIG. 11 provides a summary of source diversity, library design, hit diversity, and population bias for several generations of sulfonylurea repressor shuffling libraries L1, L2, L4, L6, L7 and resulting sequence incorporation biases. A dash (“-”) indicates no amino acid diversity introduced at that position in that library. An X indicates that the library oligonucleotides were designed to introduce complete amino acid diversity (any of 20 amino acids) at that position in that library. Residues in bold indicate bias during selection with larger font size indicating a greater degree of bias in the selected population. Residues in parentheses indicate selected mutations. The phylogenetic diversity pool was derived from a broad family of 34 tetracycline repressor sequences.

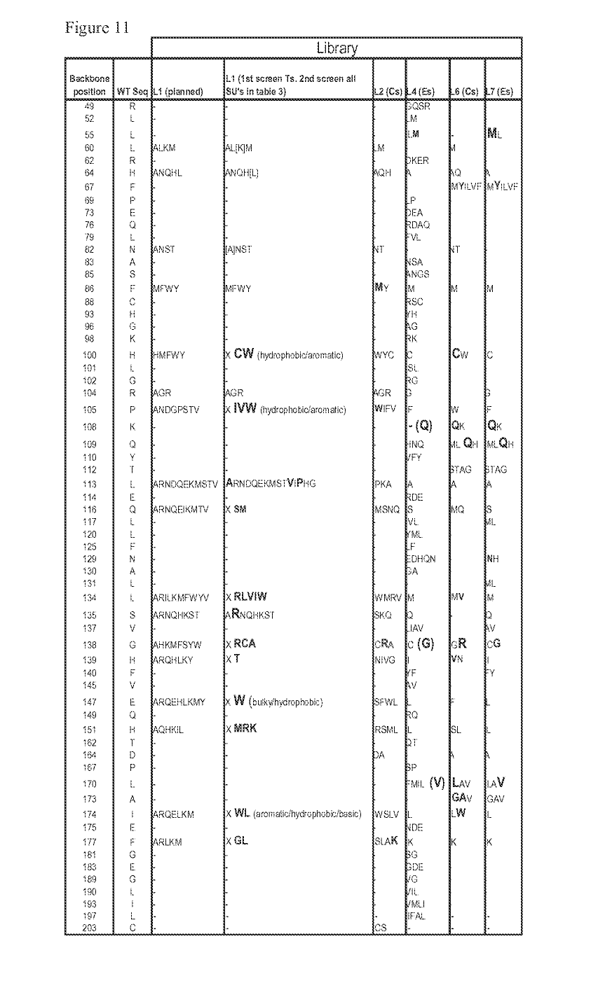

FIG. 12 provides a summary of source diversity, library design, hit diversity, and population bias for several generations of sulfonylurea repressor shuffling libraries Description of libraries L10, L11, L12, L13, L15 and resulting sequence incorporation biases. A dash (“-”) indicates no amino acid diversity introduced at that position in that library. An X indicates that the library oligonucleotides were designed to introduce complete amino acid diversity (any of 20 amino acids) at that position in that library. Residues in bold indicate bias during selection with larger font size indicating a greater degree of bias in the selected population. Residues in parentheses indicate selected mutations.

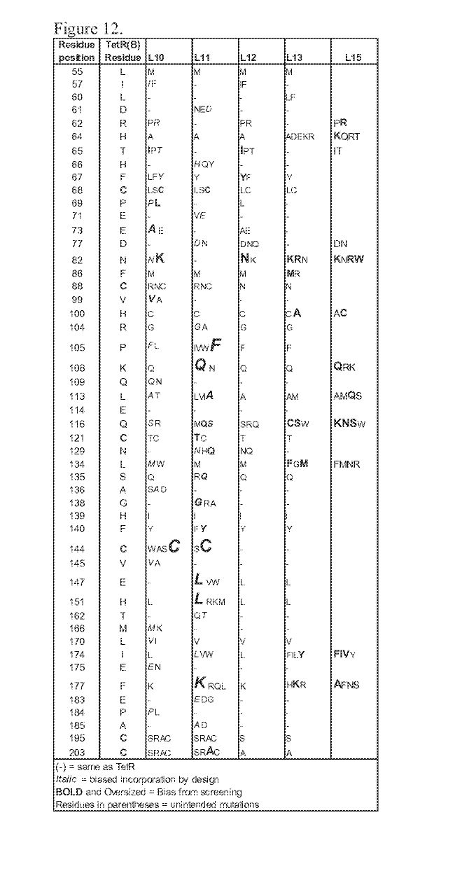

FIG. 13 provides B-galactosidase assays of hits from saturation mutagenesis at position D178 in CsR.

FIG. 14 shows the proximity of residues L131 and T134 to the sulfonylurea differentiating side groups of Chlorsulfuron bound CsR(CsL4.2-20).

FIG. 15 shows the relative position and orientations of the bound ligands tetracycline-Mg²⁺ (black), chlorsulfuron (gray with black outline), and ethametsulfuron (white with black outline), following superposition of their respective repressor structures. The herbicides occupy the same overall binding pocket, but have dramatically different conformations within it.

FIG. 16 shows the ethametsulfuron (white carbons) binding pocket from the ethametsulfuron repressor EsR(L11-C6) crystal structure. The two subunits of the dimeric repressor are shown in diagonal stripes patter, and cross hatch pattern, respectively. Straight, dashed black lines represent hydrogen bonds or ionic interactions, while semicircular dashes represent non-polar interactions. The degree of hydrophobic and hydrogen bonding interactions between TetR/Tet and EsR/Es are similar, but the precise interactions are quite different.

FIG. 17 shows interactions between ethametsulfuron (black) and the ethametsulfuron repressor EsR(L11-C6) in the crystal structure. The two subunits of the dimeric repressor are colored white (with black outline) and gray (with black outline), respectively. Straight, dashed black lines represent hydrogen bonds or ionic interactions, while semicircular dashes represent non-polar interactions.

FIG. 18 shows the chlorsulfuron (white carbons) binding pocket from the chlorsulfuron repressor CsR(L4.2-20) crystal structure. The two subunits of the dimeric repressor are shown in diagonal stripes pattern, and cross hatch pattern, respectively. Straight, dashed black lines represent hydrogen bonds or ionic interactions.

FIG. 19 shows interactions between chlorsulfuron (black) and the chlorsulfuron repressor CsR(L4.2-20) in the crystal structure. The two subunits of the dimeric repressor are colored white (with black outline) and gray (with black outline), respectively. Straight, dashed black lines represent hydrogen bonds or ionic interactions, while semicircular dashes represent non-polar interactions.6

### DETAILED DESCRIPTION

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### I. Sulfonylurea-Dependent Stabilization Domains

Polypeptides having a sulfonylurea (SU)-dependent stabilization domain are provided. As used herein, a polypeptide having a SU-dependent stabilization domain comprises a polypeptide whose stability is influenced by the presence or the absence of an effective concentration of a SU ligand. In specific embodiments, the polypeptide having the SU-dependent stabilization domain will have increased protein stability in the presence of an effective amount of the SU.

Protein stability can be assayed for in many ways, including, for example measuring for a modulation in the concentration and/or activity of the polypeptide of interest. In general, an increase in protein stability can be measured by an increase in the concentration and/or activity of the protein by at least 1%, 5%, 10%, 20%, 30%, 40%, 50%, 60%, 70%, 80%, or 90% relative to an appropriate control that was not exposed to the effective amount of the SU ligand. Alternatively, an increase in protein stability can be measured by an increase in the concentration and/or activity of the protein by at least 1 fold, 2 fold, 3 fold, 5 fold, 10 fold, 20 fold, 30 fold, 40 fold, 50 fold, 60 fold, 70 fold, 80 fold, 90 fold, 100 fold or greater relative to an appropriate control that was not exposed to the effective amount of the SU ligand.

In specific embodiments, the SU-dependent stabilization domain can comprise a ligand binding domain of a SU chemically-regulated transcriptional regulator, wherein the ligand binding domain comprises at least one destabilization mutation. As used herein, a “destabilization mutation” comprises an alteration in the amino acid sequence that results in the polypeptide having the alteration to have an increased stability in the presence of an effective concentration of a SU ligand, when compared to the stability of the polypeptide lacking the mutation.

Various SU chemically-regulated transcriptional regulators are known. See, for example WO2010/062518 and U.S. application Ser. No. 13/086,765, filed Apr. 14, 2012, each of which is herein incorporated by reference in their entirety. Non-limiting examples of SU chemically-regulated transcriptional regulators are set forth in SEQ ID NO:3-419, 863-870, 884-889, and 1193-1568 and 1949-2110 and their ligand binding domain is found at amino acids 47-207 of each of these SEQ ID NOs. Thus, in one embodiment, a SU-dependent stabilization domain comprises a ligand binding domain from a SU chemically-regulated transcriptional regulator, wherein the ligand binding domain has at least 1, 2, 3, 4, 5, 6 or more destabilization mutations.

Thus, in some embodiments, the SU-dependent stabilization domain comprising the ligand binding domain of a SU chemically-regulated transcriptional regulator comprises at least 70%, 75%, 80%, 85%, 90%, 91%, 92%, 93%, 94%, 95%, 96%, 97%, 98%, or 99% sequence identity to the ligand binding domain of an amino acid sequence set forth in any one of SEQ ID NO:3-419, 863-870, 884-889 and/or 1193-1568 and 1949-2110, wherein said polypeptide further comprises at least one destabilization mutation. In some examples the global alignment method uses the GAP algorithm with default parameters for an amino acid sequence % identity and % similarity using GAP Weight of 8 and Length Weight of 2, and the BLOSUM62 scoring matrix.

Non-limiting examples of destabilization mutations that can be made in the ligand binding domain of a SU chemically-regulated transcriptional regulator include, for example, altering the glycine as position 96 to arginine (G96R) with the amino acid position being referenced being relative to the amino acid sequence of L13-2-46(B10) the SU chemically regulated transcriptional repressor set forth in SEQ ID NO: 405. Also, double mutant arginine 94 to proline combined with valine 99 to glutamate (R94P/V99E) can be included in this class (Resch M, et al. (2008) A protein functional leap: How a single mutation reverses the function of the transcription regulator TetR. Nucleic Acids Res 36:4390-440, which is herein incorporated by reference in its entirety). Thus, when one or more of these destabilization mutations are present in the ligand binding domain of the SU chemically-regulated transcriptional regulator, the polypeptide has a decreased stability in the absence of the SU ligand and an increased stability in the presence of an effective amount of the SU ligand.

In other embodiments, the SU-dependent stabilization domain can comprise a DNA binding domain of a SU chemically-regulated transcriptional regulator, wherein the DNA binding domain comprises at least one destabilization mutation. Various SU chemically-regulated transcriptional regulators are known. See, for example WO2010/062518 and U.S. application Ser. No. 13/086,765, all of which are herein incorporated by reference. Non-limiting examples of SU chemically-regulated transcriptional regulators are set forth in SEQ ID NO:3-419, 863-870, 884-889, 1193-1568 and/or 1949-2110 and/or and their DNA binding domain is found at amino acids 1-46 of each of these SEQ ID NOs. Thus, in one embodiment, a SU-dependent stabilization domain comprises a DNA binding domain from a SU chemically-regulated transcriptional regulator, wherein the DNA binding domain has at least 1, 2, 3, 4, 5, 6 or more destabilization mutations.

Thus, in some embodiments, the SU-dependent stabilization domain comprising the DNA binding domain of the SU chemically-regulated transcriptional regulator comprises at least 70%, 75%, 80%, 85%, 90%, 91%, 92%, 93%, 94%, 95%, 96%, 97%, 98%, or 99% sequence identity to the DNA binding domain of an amino acid sequences sequence set forth in any one of SEQ ID NO:3-419, 863-870, 884-889, 1193-1568 and/or 1949-2110 wherein said polypeptide further comprises at least one destabilization mutation. In some examples the global alignment method uses the GAP algorithm with default parameters for an amino acid sequence % identity and % similarity using GAP Weight of 8 and Length Weight of 2, and the BLOSUM62 scoring matrix.

Non-limiting examples of destabilization mutations that can be made in the DNA binding domain of a SU chemically-regulated transcriptional repressor include, for example, altering the leucine as position 17 to glycine (L17G), the isoleucine at position 22 to aspartic acid (I22D), and/or altering the leucine at position 30 to aspartic acid (L30D) or leucine at position 34 to aspartic acid (L34D). See, Reichheld S E, Davidson A R (2006) Two-way interdomain signal transduction in tetracycline repressor. J Mol Biol 361:382-389, which is herein incorporated by reference in its entirety). The amino acid position being referenced is relative to the amino acid sequence of the SU chemically regulated transcriptional repressor set forth in SEQ ID NO: 405. Thus, when one or more of these destabilization mutations are present in the DNA binding domain of the SU chemically-regulated transcriptional regulator, the polypeptide has a decreased stability in the absence of the SU ligand and an increased stability in the presence of an effective amount of the SU ligand.

In other embodiments, the SU-dependent stabilization domain comprises both the DNA binding domain and the SU ligand binding domain of the SU chemically-regulated transcriptional regulator. As such, any combination of the destabilization mutations of the DNA binding domain and/or the ligand binding domain can be used to produce a polypeptide having a SU-dependent stabilization domain. In specific embodiments, a SU dependent stabilization domain comprises a combination of any one of the L17G, I22D and/or G96R mutation.

Thus, in some embodiments, the SU-dependent stabilization domain comprises a polypeptide having at least 70%, 75%, 80%, 85%, 90%, 91%, 92%, 93%, 94%, 95%, 96%, 97%, 98%, or 99% sequence identity to the full length SU chemically-regulated transcriptional regulator set forth in any one of SEQ ID NO:3-419, 863-870, 884-889, 1193-1568 and/or 1949-2110, wherein said polypeptide further comprises at least one destabilization mutation and thus increases the stability of the polypeptide in the presence of an effective concentration of the SU ligand. When a SU chemically-regulated transcriptional regulator is employed as a SU-dependent stabilization domain, the SU chemically-regulated transcriptional regulator can continue to retain transcriptional regulatory activity, and in some embodiments, the transcriptional regulatory activity is not retained. In some examples the global alignment method uses the GAP algorithm with default parameters for an amino acid sequence % identity and % similarity using GAP Weight of 8 and Length Weight of 2, and the BLOSUM62 scoring matrix.

In non-limiting embodiments, the SU-dependent stabilization domain can have an equilibrium binding constant for a sulfonylurea compound greater than 0.1 nM and less than 10 μM. In some examples, the SU-dependent stabilization domain has an equilibrium binding constant for a sulfonylurea compound of at least 0.1 nM, 0.5 nM, 1 nM, 10 nM, 50 nM, 100 nM, 250 nM, 500 nM, 750 nM, 1 μM, 5 μM, 7 μM but less than 10 μM. In other examples, the SU-dependent stabilization domain has an equilibrium binding constant for a sulfonylurea compound of at least 0.1 nM, 0.5 nM, 1 nM, 10 nM, 50 nM, 100 nM, 250 nM, 500 nM, 750 nM but less than 1 μM. In some embodiments, the SU-dependent stabilization domain has an equilibrium binding constant for a sulfonylurea compound greater than 0 nM, but less than 0.1 nM, 0.5 nM, 1 nM, 10 nM, 50 nM, 100 nM, 250 nM, 500 nM, 750 nM, 1 μM, 5 μM, 7 μM or 10 μM. In some examples, the sulfonylurea compound is a chlorsulfuron, an ethametsulfuron, a metsulfuron, a sulfometuron, a tribenuron, a chlorimuron, a nicosulfuron, a rimsulfuron and/or a thifensulfuron.

i. Reverse SU-Chemically Regulated Transcriptional Repressors (revSuRs) Having at Least One Destabilization Mutation

In some embodiments, the SU-dependent stabilization domain comprises a reverse SU chemically-regulated transcription repressor (revSuR), having at least one destabilization domain, such that the destabilization mutation increases the stability of the polypeptide in the presence of an effective concentration of the SU ligand.

As used herein, a “reverse SU chemically-regulated transcriptional repressor” or “revSuR” comprises a polypeptide that contains a DNA binding domain and a SU ligand binding domain. In the absence of the SU ligand, the revSuR is both unstable as well as unable to bind an operator of a ligand responsive promoter and repress the activity of the promoter, and thereby allows for the expression of the polynucleotide operably linked to the promoter. In the presence of an effective concentration of the SU chemical ligand, the revSuR is stabilized. The ligand-bound revSuR can then bind the operator of a ligand responsive promoter and repress transcription. Variants and fragments of a revSuR chemically-regulated transcriptional repressor will retain this activity, and thereby repress transcription in the presence of the SU ligand.

Non-limiting examples of revSuRs are set forth in WO2010/062518 and U.S. application Ser. No. 13/086,765, herein incorporated by reference. Also, SEQ ID NO:412-419 or active variants and fragments thereof comprise revSuR polynucleotides and the polypeptides they encode. These various revSuRs can be altered to contain a SU-dependent stabilization domain comprising at least one destabilization mutation, such that the revSuR is unstable in the absence of the effective amount of the SU ligand. As such, further provided are polynucleotides and polypeptides comprising any one of SEQ ID NO:412-419 or a sequence having at least 70%, 75%, 80%, 85%, 90%, 91%, 92%, 93%, 94%, 95%, 96%, 97%, 98% or 99% sequence identity to any one of SEQ ID NOS: 412-419, wherein said sequence comprises one or more destabilization mutations. revSuR polypeptides or active variants thereof are thus unstable in the absence of an effective amount of SU ligand and, in the presence of the an effective amount of SU ligand, the revSuR decreases transcriptional activation activity.

In some examples the rev(SuR) polypeptide is selected from the group consisting of SEQ ID NO:412-419 and further comprises at least one destabilization mutation, and the sulfonylurea compound is selected from the group consisting of a chlorsulfuron, an ethametsulfuron, a metsulfuron, a sulfometuron, a tribenuron, a chlorimuron, a nicosulfuron, a rimsulfuron and a thifensulfuron.

In some examples, the rev SuR having at least one destabilization mutation has an equilibrium binding constant for an operator sequence greater than 0.1 nM and less than 10 μM. In some examples the rev SuR having at least one destabilization mutation has an equilibrium binding constant for an operator sequence of at least 0.1 nM, 0.5 nM, 1 nM, 10 nM, 50 nM, 100 nM, 250 nM, 500 nM, 750 nM, 1 μM, 5 μM, 7 μM but less than 10 μM. In some examples, the revSuR having at least one destabilization mutation has an equilibrium binding constant for an operator sequence of at least 0.1 nM, 0.5 nM, 1 nM, 10 nM, 50 nM, 100 nM, 250 nM, 500 nM, 750 nM but less than 1 μM. In some examples the revSuR having at least one destabilization mutation has an equilibrium binding constant for an operator sequence greater than 0 nM, but less than 0.1 nM, 0.5 nM, 1 nM, 10 nM, 50 nM, 100 nM, 250 nM, 500 nM, 750 nM, 1 μM, 5 μM, 7 μM or 10 μM. In some examples, the operator sequence is a Tet operator sequence. In some examples, the Tet operator sequence is a TetR(A) operator sequence, a TetR(B) operator sequence, a TetR(D) operator sequence, TetR(E) operator sequence, a TetR(H) operator sequence, or a functional derivative thereof.

In specific embodiments, a transcriptional activation domain (denoted herein as TAD or TA) can be fused in frame to the revSuR and thereby influence the activity of the revSuR. In such instances, the binding of the revSuR-TAD to the operator will result in transcriptional activation of the operably linked sequence of interest. Employing such transcriptional activation domains is known. For example, the VP16 transcriptional domain can be operably linked to the revSuR sequence and thereby allow for transcriptional activation in the presence of the SU ligand. See, for example, Gossen et al. (1995) Science 268:1766-1769. A revSuR-TAD having at least one destabilization mutation is unstable in the absence of an effective concentration of a SU ligand. In the presence of an effective concentration of an SU ligand, the revSuR-TAD having the at least one destabilization mutation is stable and the polypeptide can then increase transcription from a cognate ligand responsive promoter.

In some examples, the rev(SuR)-TAD polypeptide comprises a revSuR selected from the group consisting of SEQ ID NO:412-419 and further comprises at least one destabilization mutation and a TAD, and the sulfonylurea compound is selected from the group consisting of a chlorsulfuron, an ethametsulfuron, a metsulfuron, a sulfometuron, a tribenuron, a chlorimuron, a nicosulfuron, a rimsulfuron and a thifensulfuron.

Thus, a revSuR can be designed to either activate transcription or repress transcription. By “activate transcription” is intended an increase of transcription of a given polynucleotide. An increase in transcription can comprise any statistically significant increase including, an increase of at least 1%, 5%, 10%, 20%, 30%, 40%, 50%, 60%, 70%, 80%, 90%, 100%, 150%, 200% or greater or at least a 1, 2, 3, 4, 5, 6, 7, 8, 9, or 10 fold increase. A decrease in transcription can comprise any statistically significant decrease including, a decrease of at least 1%, 5%, 10%, 20%, 30%, 40%, 50%, 60%, 70%, 80%, 90%, 100%, 150%, 200% or greater or at least a 1, 2, 3, 4, 5, 6, 7, 8, 9, or 10 fold decrease.

### II. Recombinant Constructs Comprising the SU-Dependent Stabilization Domain

ii. Fusion Proteins Comprising a SU-Dependent Stabilization Domain Operably Linked to a Polypeptide of Interest

Polypeptides comprising a SU-dependent stabilization domain fused in frame to a polypeptide of interest are provided, as are the polynucleotides encoding the same. In such instances, the fusion protein would have an increased stability in the presence of an effective amount of the SU ligand and thereby show an increase in the level of the fusion protein. In the absence of the effective amount of the SU ligand, the fusion protein would be less stable and thereby result in a decreased level of the fusion protein.

Any SU-dependent stabilization domain can be employed in the fusion proteins and polynucleotides encoding the same, including, for example, the ligand binding domain of a SU chemically-regulated transcriptional regulator with at least one destabilization mutation, the DNA binding domain of a SU chemically-regulated transcriptional regulator with at least one destabilization mutation, a SuR having at least one destabilization mutation, a revSuR having at least one destabilization domain, or a revSuR-TAD having at least one destabilization domain. Each of these forms of SU-dependent stabilization domains are discussed in further detail elsewhere herein.

In general, the fusion protein comprising the SU-dependent stabilization domain may be fused in frame to: an enzyme involved in metabolism, biosynthesis and the like; a transcription factor for modulation of any phenotypic aspect of a cell or organism; a sequence specific nuclease designed for stimulating targeted mutagenesis, site specific integration and/or homologous recombination of donor DNA; or any other protein for which it is desired to regulate the steady state level of.

In one embodiment, the fusion protein comprising the SU-dependent stabilization domain fused in frame to a polypeptide of interest further comprises an intein. As used herein, an “intein” comprises a peptide that is excised from a polypeptide and the flanking “extein” regions of the intein are ligated together. When employed with a fusion protein disclosed herein, the intein is designed such that the flanking extein regions (i.e., the polypeptide of interest and the SU stabilization domain) are not rejoined. Thus, the intein retains cleavage activity, but has reduced ability or no ability to religate the extein sequences. Thus, the polypeptide of interest can be freed from the SU-dependent stabilization domain. In this regard there would be no adverse effect of having a fusion protein as it would be released from the union leaving the target protein in its native state. See, for example, Buskirk (2004) PNAS 101:10505-10510 and NEB Catalog #E6900S for TM PACT™-CN.

ii. Promoters for Expression of the Fusion Proteins Comprising the SU-Dependent Stabilization Domain

The polynucleotide encoding the fusion protein comprising the SU-dependent stabilization domain can be operably linked to a promoter that is active in any host cell of interest. In specific embodiments, the promoter is active in a plant. Various promoters can be employed and non-limiting examples are set forth elsewhere herein. Briefly, the fusion protein can be operably linked to a constitutive promoter, an inducible promoter, tissue-preferred promoter, or a ligand responsive promoter. In specific embodiments, the fusion protein comprising the SU-dependent stabilization domain is operably linked to a non-constitutive promoter, including, but not limited to, a tissue-preferred promoter, an inducible promoter, a ligand responsive promoter, a developmental stage preferred promoter, or a promoter having more than one of these properties. In some examples expression of the polynucleotide of interest is primarily regulated in roots, leaves, stems, flowers, silks, anthers, pollen, meristem, germline, seed, endosperm, embryos, or progeny.

When the fusion protein comprises a revSuR-TAD having at least one destabilization mutation fused to a polypeptide of interest, the polynucleotide encoding the same can be operably linked to a ligand responsive promoter, and thereby allowing the revSuR-TAD, in the presence of an effective amount of SU ligand, to increase its own expression. Thus, in specific embodiments, the fusion protein comprising the revSuR-TAD can be operably linked to a ligand responsive promoter comprising at least one, two, three or more operators (including a tet operator, such as that set forth in SEQ ID NO:848 or an active variant or fragment thereof) regulating expression of said repressor. The regulated promoter could be a repressible promoter regulated additionally by a non-destabilized SuR or a hybrid repressible-activatable promoter regulated by both a non-destabilized SuR as well as a destabilized revSuR-TAD. Non-limiting examples of ligand responsive promoters for expression of the chemically-regulated transcriptional repressor, include the ligand responsive promoters set forth in SEQ ID NO:885, 856, 857, 858, 859, or 860 or active variants and fragments thereof.

In another example the promoter may be both activated by revSuR-TAD in the presence of SU and repressed in the absence of SU by a co-expressed trans-dominant SuR-TR that recruits the histone deacetylase complex and induces transcriptional silence. In this strategy the SuR chosen for activation and the one chosen for repression would lack hetero-dimerization capacity (Sabine Freundlieb et al. (1999) J Gene Med. 1:4-12, which is herein incorporated by reference in its entirety).

In yet another example, the regulated promoter could be a hybrid repressible-activatable promoter regulated by both a non-destabilized SuR as well as a destabilized revSuR-TAD. In this case, there could be two sets of operators sequences: one upstream of the promoter acting to recruit revSuR-TA for promoter activation and then a second set of modified operators located in and around the TATA box and transcriptional start sites that would be bound only by an SuR mutated in the DNA binding domain to recognize these modified operators. The revSuR-TAD and SuR* would also have to be designed as to not heterodimerize as their co-expression would likely lead to non-functional activators and repressors. Previously it has been shown that tet operators mutated at positions 4 and 6 relative to the center of the dyad core disallow binding by TetR and that compensatory mutations in TetR re-enable binding and functional repression from these mutated operators. Co-expression of wildtype and mutated TetR repressors have been shown to independently regulate genes from wildtype and mutant operators (Gene regulation by tetracyclines: Constraints of resistance regulation in bacteria shape TetR for application in eukaryotes. Christian Berens and Wolfgang Hillen. Eur. J. Biochem. 270, 3109-3121 (2003)). Thus it may be possible to design a promoter for both activation and repression using the SuR system.

iii. Polypeptides of Interest

Any polypeptide of interest can be employed in the fusion proteins discussed above, as well as, the encoding polynucleotide sequence in the corresponding DNA construct. Such polypeptides of interest are discussed in detail elsewhere herein.

### III. The SU-Dependent Stabilization Domain in a Chemical Gene-Switch and Methods of Use

The polypeptide comprising the SU-dependent stabilization domain can further be employed in a chemical-gene switch system. The chemical-gene switch employing a SU-dependent stabilization domain comprises at least two components. The first component comprises a first recombinant construct comprising a first promoter operably linked to a SU chemically-regulated transcriptional regulator comprising a revSuR having a TAD, wherein the revSuR comprises a destabilization mutation. The second component comprises a second recombinant construct comprising a first ligand responsive promoter comprising at least 1, 2, 3, 4, 5, 6, 7, 8, 9 10 or more cognate operators for the revSuR operably linked to a polynucleotide of interest. In such a system, in the absence of an effective amount of the SU ligand, the revSuR is unstable and the polypeptide does not accumulate in the cell. As such, the polynucleotide of interest is transcribed at its base-line level. In the presence of an effective concentration of a SU ligand, the revSuR-TAD is stabilized and thus, an increase in the level of the revSuR-TAD occurs. The revSuR-TAD can then increase the level of transcription from the first ligand responsive promoter

As explained in further detail herein, the activity of the chemical-gene switch can be controlled by selecting the combination of elements used in the switch. These include, but are not limited to, the type of promoter operably linked to the revSuR-TAD having the destabilization mutations, the ligand responsive promoter operably linked to the polynucleotide of interest, the TAD operably linked to the revSuR, and the polynucleotide of interest. Further control is provided by selection, dosage, conditions, and/or timing of the application of the SU ligand.

i. Promoters for the Expression of the RevSuR-TAD Comprising the Destabilization Mutation

When employed in a chemical-gene switch, the polynucleotide encoding the revSuR-TAD comprising the at least one destabilization mutation is operably linked to a promoter that is active in a host cell of interest, including, for example, a plant cell. Various promoters can be employed and non-limiting examples are set forth elsewhere herein. Briefly, the polynucleotide encoding the revSuR-TAD comprising the at least one destabilization mutation can be operably linked to a constitutive promoter, an inducible promoter, a tissue-preferred promoter, or a ligand responsive promoter. In specific embodiments, the polynucleotide encoding the revSuR-TAD is operably linked to a non-constitutive promoter, including but not limited to a tissue-preferred promoter, an inducible promoter, a ligand responsive promoter, a developmental stage preferred promoter, or a promoter having more than one of these properties. In some examples expression of the polynucleotide encoding the revSuR-TAD is primarily regulated in roots, leaves, stems, flowers, silks, anthers, pollen, meristem, germline, seed, endosperm, embryos, or progeny.

In other embodiments, the revSuR-TAD having the at least one destabilization mutation can be operably linked to a ligand responsive promoter, thus allowing the chemically-regulated transcriptional repressor to auto-regulate its own expression. Thus, in specific embodiments, the polynucleotide encoding the revSuR-TAD can be operably linked to a ligand responsive promoter comprising at least one, two, three, four, five, six, seven, eight, nine, ten or more operators (including a tet operator, such as that set forth in SEQ ID NO:848 or an active variant or fragment thereof) regulating expression of the revSuR-TAD. Non-limiting ligand responsive promoters for expression of the revSuR-TAD, include the ligand responsive promoters set forth in SEQ ID NO:848, 885, 856, 857, 858, 859, or 860 or active variants and fragments thereof.

ii. Promoters for Expression of the Polynucleotide of Interest

In the chemical-gene switch system, the polynucleotide of interest is operably linked to a ligand responsive promoter active in the host cell or plant. Various ligand responsive promoters that can be used to express the polynucleotide of interest are discussed in detail elsewhere herein.

### IV. Polynucleotides/Polypeptides of Interest.

Any polynucleotide or polypeptide of interest either in the fusion protein comprising the SU stabilization domain or in the chemical-gene switch system can be employed in the various methods and compositions disclosed herein. In specific embodiments, expression of the polynucleotide of interest alters the phenotype and/or genotype of the plant. An altered genotype includes any heritable modification to any sequence in a plant genome. An altered phenotype includes any scenario wherein a cell, tissue, plant, and/or seed exhibits a characteristic or trait that distinguishes it from its unaltered state. Altered phenotypes included, but are not limited to, a different growth habit, altered flower color, altered relative maturity, altered yield, altered fertility, altered flowering time, altered disease tolerance, altered insect tolerance, altered herbicide tolerance, altered stress tolerance, altered water tolerance, altered drought tolerance, altered seed characteristics, altered morphology, altered agronomic characteristic, altered metabolism, altered gene expression profile, altered ploidy, altered crop quality, altered forage quality, altered silage quality, altered processing characteristics, and the like.

Polynucleotides of interest are reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increase, the choice of genes for transformation will change accordingly. General categories of genes of interest include, for example, those genes involved in information, such as zinc fingers, those involved in communication, such as kinases, and those involved in housekeeping, such as heat shock proteins. More specific categories of transgenes, for example, include genes encoding important traits for agronomics, insect resistance, disease resistance, herbicide resistance, sterility, grain characteristics, and commercial products. Genes of interest include, generally, those involved in oil, starch, carbohydrate, or nutrient metabolism, as well as, those affecting kernel size, sucrose loading, and the like.

In still other embodiments, the polynucleotide of interest may be any sequence of interest, including but not limited to sequences encoding a polypeptide, encoding an mRNA, encoding an RNAi precursor, encoding an active RNAi agent, a miRNA, an antisense polynucleotide, a ribozyme, a fusion protein, a replicating vector, a screenable marker, and the like. Expression of the polynucleotide of interest may be used to induce expression of an encoding RNA and/or polypeptide, or conversely to suppress expression of an encoded RNA, RNA target sequence, and/or polypeptide. In specific examples, the polynucleotide sequence may a polynucleotide encoding a plant hormone, plant defense protein, a nutrient transport protein, a biotic association protein, a desirable input trait, a desirable output trait, a stress resistance gene, a disease/pathogen resistance gene, a male sterility, a developmental gene, a regulatory gene, a DNA repair gene, a transcriptional regulatory gene or any other polynucleotide and/or polypeptide of interest.

Agronomically important traits such as oil, starch, and protein content can be genetically altered in addition to using traditional breeding methods. Modifications include increasing content of oleic acid, saturated and unsaturated oils, increasing levels of lysine and sulfur, providing essential amino acids, and also modification of starch. Hordothionin protein modifications are described in U.S. Pat. Nos. 5,703,049, 5,885,801, 5,885,802, and 5,990,389, herein incorporated by reference. Another example is lysine and/or sulfur rich seed protein encoded by the soybean 2S albumin described in U.S. Pat. No. 5,850,016, and the chymotrypsin inhibitor from barley, described in Williamson et al. (1987) Eur. J. Biochem. 165:99-106, the disclosures of which are herein incorporated by reference.

Derivatives of the coding sequences can be made by site-directed mutagenesis to increase the level of preselected amino acids in the encoded polypeptide. For example, the gene encoding the barley high lysine polypeptide (BHL) is derived from barley chymotrypsin inhibitor, U.S. application Ser. No. 08/740,682, filed Nov. 1, 1996, and WO 98/20133, the disclosures of which are herein incorporated by reference. Other proteins include methionine-rich plant proteins such as from sunflower seed (Lilley et al. (1989) Proceedings of the World Congress on Vegetable Protein Utilization in Human Foods and Animal Feedstuffs, ed. Applewhite (American Oil Chemists Society, Champaign, Ill.), pp. 497-502; herein incorporated by reference); corn (Pedersen et al. (1986) J. Biol. Chem. 261:6279; Kirihara et al. (1988) Gene 71:359; both of which are herein incorporated by reference); and rice (Musumura et al. (1989) Plant Mol. Biol. 12:123, herein incorporated by reference). Other agronomically important genes encode latex, Floury 2, growth factors, seed storage factors, and transcription factors.

Insect resistance genes may encode resistance to pests that have great yield drag such as rootworm, cutworm, European Corn Borer, and the like. Such genes include, for example, Bacillus thuringiensis toxic protein genes (U.S. Pat. Nos. 5,366,892; 5,747,450; 5,736,514; 5,723,756; 5,593,881; and Geiser et al. (1986) Gene 48:109); and the like.

Genes encoding disease resistance traits include detoxification genes, such as against fumonosin (U.S. Pat. No. 5,792,931); avirulence (avr) and disease resistance (R) genes (Jones et al. (1994) Science 266:789; Martin et al. (1993) Science 262:1432; and Mindrinos et al. (1994) Cell 78:1089); and the like.

Herbicide resistance traits may include genes coding for resistance to herbicides that act to inhibit the action of acetolactate synthase (ALS), in particular the sulfonylurea-type herbicides (e.g., the acetolactate synthase (ALS) gene containing mutations leading to such resistance, in particular the S4 and/or Hra mutations), genes coding for resistance to herbicides that act to inhibit action of glutamine synthase, such as phosphinothricin or basta (e.g., the bar gene); glyphosate (e.g., the EPSPS gene and the GAT gene; see, for example, U.S. Publication No. 20040082770 and WO 03/092360); or other such genes known in the art. The bar gene encodes resistance to the herbicide basta, the nptll gene encodes resistance to the antibiotics kanamycin and geneticin, and the ALS-gene mutants encode resistance to the herbicide chlorsulfuron.

Sterility genes can also be encoded in an expression cassette and provide an alternative to physical detasseling. Examples of genes used in such ways include male tissue-preferred genes and genes with male sterility phenotypes such as QM, described in U.S. Pat. No. 5,583,210. Other genes include kinases and those encoding compounds toxic to either male or female gametophytic development.

The quality of grain is reflected in traits such as levels and types of oils, saturated and unsaturated, quality and quantity of essential amino acids, and levels of cellulose. In corn, modified hordothionin proteins are described in U.S. Pat. Nos. 5,703,049, 5,885,801, 5,885,802, and 5,990,389.

Commercial traits can also be encoded on a gene or genes that could increase for example, starch for ethanol production, or provide expression of proteins. Another important commercial use of transformed plants is the production of polymers and bioplastics such as described in U.S. Pat. No. 5,602,321. Genes such as β-Ketothiolase, PHBase (polyhydroxyburyrate synthase), and acetoacetyl-CoA reductase (see Schubert et al. (1988) J. Bacteriol. 170:5837-5847) facilitate expression of polyhyroxyalkanoates (PHAs).

Exogenous products include plant enzymes and products as well as those from other sources including prokaryotes and other eukaryotes. Such products include enzymes, cofactors, hormones, and the like. The level of proteins, particularly modified proteins having improved amino acid distribution to improve the nutrient value of the plant, can be increased. This is achieved by the expression of such proteins having enhanced amino acid content.

Additional polypeptide of interest include, for example, polypeptides such as various site specific recombinases and systems employing the same. See, for example, WO99/25821, WO99/25854, WO99/25840, WO99/25855, and WO99/25853, all of which are herein incorporated by reference. Other sequences of interest can include various meganucleases to target polynucleotides are set forth in WO 2009/114321 (herein incorporated by reference), which describes “custom” meganucleases. See, also, Gao et al. (2010) Plant Journal 1:176-187. Additional sequence of interest that can be employed, include but are not limited to ZnFingers, meganucleases, and, TAL nucleases. See, for example, WO2010079430, WO2011072246, and US20110201118, each of which is herein incorporated by reference in their entirety.

V. Sequences that Confers Tolerance to SU Ligand

As discussed elsewhere herein, a variety of SU ligands can be employed in the methods and compositions disclosed herein. It is recognized that host cell, the plant or plant part when exposed to the SU ligand should remain tolerant to the SU ligand employed. As used herein, “SU ligand-tolerant” or “tolerant” or “crop tolerance” or “herbicide-tolerant” or “sulfonylurea-tolerant” in the context of chemical-ligand treatment is intended that a host cell (i.e., a plant or plant cell) treated with the SU ligand will show no significant damage following the treatment in comparison to a host cell (i.e., a plant or plant part) not exposed the SU chemical ligand. A host cell (i.e., a plant) may be naturally tolerant to the SU ligand, or the host cell (i.e, the plant) may be tolerant to the SU ligand as a result of human intervention such as, for example, by the use of a recombinant construct, plant breeding or genetic engineering. Thus, the host cell (i.e., the plants) employed in the various methods disclosed herein can comprise a native or a heterologous sequence that confers tolerance to the sulfonylurea compound.

In one embodiment, the host cell, the plant or plant cell comprises a sulfonylurea-tolerant polypeptide. As used herein, a “sulfonylurea-tolerant polypeptide” comprises any polypeptide which when expressed in a host cell or a plant or a plant cell confers tolerance to at least one sulfonylurea. Sulfonylurea herbicides inhibit growth of higher plants by blocking acetolactate synthase (ALS), also known as, acetohydroxy acid synthase (AHAS). Plants containing particular mutations in ALS (e.g., the S4 and/or HRA mutations) are tolerant to sulfonylurea herbicides. The production of sulfonylurea-tolerant plants is described more fully in U.S. Pat. Nos. 5,605,011; 5,013,659; 5,141,870; 5,767,361; 5,731,180; 5,304,732; 4,761,373; 5,331,107; 5,928,937; and 5,378,824; and international publication WO 96/33270, which are incorporated herein by reference in their entireties for all purposes. The sulfonylurea-tolerant polypeptide can be encoded by, for example, the SuRA or SuRB locus of ALS. In specific embodiments, the ALS inhibitor-tolerant polypeptide comprises the C3 ALS mutant, the HRA ALS mutant, the S4 mutant or the S4/HRA mutant or any combination thereof. Different mutations in ALS are known to confer tolerance to different herbicides and groups (and/or subgroups) of herbicides; see, e.g., Tranel and Wright (2002) Weed Science 50:700-712. See also, U.S. Pat. Nos. 5,605,011, 5,378,824, 5,141,870, and 5,013,659, each of which is herein incorporated by reference in their entirety. The HRA mutation in ALS finds particular use in one embodiment. The mutation results in the production of an acetolactate synthase polypeptide which is resistant to at least one sulfonylurea compound in comparison to the wild-type protein. As the HRA mutation provides resistance to both SUs and imidazolinones, the use of the HRA mutation allows for the use of a selectable marker that does not trigger the induction response.

A SU ligand does not “significantly damage” a host cell, a plant or plant cell when it either has no effect on the host cell or plant or when it has some effect on the host cell or the plant from which the host cell or the plant later recovers, or when it has an effect which is detrimental but which is offset, for example, by the impact of the particular SU herbicide on weeds or the desired phenotype produced by the chemical-gene switch system. Thus, for example, a plant is not “significantly damaged by” a SU ligand treatment if it exhibits less than 50%, 40%, 30%, 25%, 20%, 15%, 10%, 9%, 8%, 7%, 6%, 5%, 4%, 3%, 2%, or 1% decrease in at least one suitable parameter that is indicative of plant health and/or productivity in comparison to an appropriate control plant (e.g., an untreated crop plant). Suitable parameters that are indicative of plant health and/or productivity include, for example, plant height, plant weight, leaf length, time elapsed to a particular stage of development, flowering, yield, seed production, and the like. The evaluation of a parameter can be by visual inspection and/or by statistical analysis of any suitable parameter. Comparison may be made by visual inspection and/or by statistical analysis. Accordingly, a crop plant is not “significantly damaged by” a herbicide or other treatment if it exhibits a decrease in at least one parameter but that decrease is temporary in nature and the plant recovers fully within 1 week, 2 weeks, 3 weeks, 4 weeks, or 6 weeks.

### VI. Promoters

As outlined in detail above, a number of promoters can be used in the various recombinant constructs disclosed herein. The promoters can be selected based on the desired outcome. Promoters of interest can be a constitutive promoter or a non-constitutive promoter. Non-constitutive promoter can include, but are not limited to, a tissue preferred promoter, an inducible promoter, a ligand responsive promoter, a developmental stage preferred promoter, or a promoter having more than one of these properties. In some examples the promoter is primarily expressed in roots, leaves, stems, flowers, silks, anthers, pollen, meristem, germline, seed, endosperm, embryos, or progeny. Non-limiting examples of promoters employed within the constructs of the chemical-gene switch are discussed in detail below.

Constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Tissue-preferred promoters can be utilized to target enhanced expression within a particular plant tissue. Tissue-preferred promoters include Yamamoto et al. (1997) Plant J. 12(2):255-265; Kawamata et al. (1997) Plant Cell Physiol. 38(7):792-803; Hansen et al. (1997)Mol. Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression.

Leaf-preferred promoters are known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6):1129-1138; and Matsuoka et al. (1993) Proc. Natl. Acad. Sci. USA 90(20):9586-9590.

Root-preferred promoters are known and can be selected from the many available from the literature or isolated de novo from various compatible species. See, for example, Hire et al. (1992) Plant Mol. Biol. 20(2):207-218 (soybean root-specific glutamine synthetase gene); Keller and Baumgartner (1991) Plant Cell 3(10):1051-1061 (root-specific control element in the GRP 1.8 gene of French bean); Sanger et al. (1990) Plant Mol. Biol. 14(3):433-443 (root-specific promoter of the mannopine synthase (MAS) gene of Agrobacterium tumefaciens); and Miao et al. (1991) Plant Cell 3(1):11-22 (full-length cDNA clone encoding cytosolic glutamine synthetase (GS), which is expressed in roots and root nodules of soybean). See also Bogusz et al. (1990) Plant Cell 2(7):633-641, where two root-specific promoters isolated from hemoglobin genes from the nitrogen-fixing nonlegume Parasponia andersonii and the related non-nitrogen-fixing nonlegume Trema tomentosa are described. The promoters of these genes were linked to a β-glucuronidase reporter gene and introduced into both the nonlegume Nicotiana tabacum and the legume Lotus corniculatus, and in both instances root-specific promoter activity was preserved. Leach and Aoyagi (1991) describe their analysis of the promoters of the highly expressed rolC and rolD root-inducing genes of Agrobacterium rhizogenes (see Plant Science (Limerick) 79(1):69-76). They concluded that enhancer and tissue-preferred DNA determinants are dissociated in those promoters. Teen et al. (1989) used gene fusion to lacZ to show that the Agrobacterium T-DNA gene encoding octopine synthase is especially active in the epidermis of the root tip and that the TR2′ gene is root specific in the intact plant and stimulated by wounding in leaf tissue, an especially desirable combination of characteristics for use with an insecticidal or larvicidal gene (see EMBO J. 8(2):343-350). The TR1′ gene, fused to nptll (neomycin phosphotransferase II) showed similar characteristics. Additional root-preferred promoters include the VfENOD-GRP3 gene promoter (Kuster et al. (1995) Plant Mol. Biol. 29(4):759-772); and rolB promoter (Capana et al. (1994) Plant Mol. Biol. 25(4):681-691. See also U.S. Pat. Nos. 5,837,876; 5,750,386; 5,633,363; 5,459,252; 5,401,836; 5,110,732; and 5,023,179.

“Seed-preferred” promoters include both “seed-specific” promoters (those promoters active during seed development such as promoters of seed storage proteins) as well as “seed-germinating” promoters (those promoters active during seed germination). See Thompson et al. (1989) BioEssays 10:108, herein incorporated by reference. Such seed-preferred promoters include, but are not limited to, Cim1 (cytokinin-induced message); cZ19B1 (maize 19 kDa zein); milps (myo-inositol-1-phosphate synthase) (see WO 00/11177 and U.S. Pat. No. 6,225,529; herein incorporated by reference). Gamma-zein is an endosperm-specific promoter. Globulin 1 (Glb-1) is a representative embryo-specific promoter. For dicots, seed-specific promoters include, but are not limited to, bean β-phaseolin, napin, β-conglycinin, soybean lectin, cruciferin, and the like. For monocots, seed-specific promoters include, but are not limited to, maize 15 kDa zein, 22 kDa zein, 27 kDa zein, gamma-zein, waxy, shrunken 1, shrunken 2, Globulin 1, etc. See also WO 00/12733, where seed-preferred promoters from end1 and end2 genes are disclosed; herein incorporated by reference.

Additional exemplary promoters include but are not limited to a 35S CaMV promoter (Odell et al. (1995) Nature 313:810-812), a S-adenosylmethionine synthase promoter (SAMS) (e.g., those disclosed in U.S. Pat. No. 7,217,858 and US2008/0026466), a Mirabilis mosaic virus promoter (e.g., Dey & Maiti (1999) Plant Mol Biol 40:771-782; Dey & Maiti (1999) Transgenics 3:61-70), an elongation factor promoter (e.g., US2008/0313776 and US2009/0133159), a banana streak virus promoter, an actin promoter (e.g., McElroy et al. (1990) Plant Cell 2:163-171), a TobRB7 promoter (e.g., Yamamoto et al. (1991) Plant Cell 3:371), a patatin promoter (e.g., patatin B33, Martin et al. (1997) Plant J 11:53-62), a ribulose 1,5-bisphosphate carboxylase promoter (e.g., rbcS-3A, see, for example Fluhr et al. (1986) Science 232:1106-1112, and Pellingrinischi et al. (1995) Biochem Soc Trans 23:247-250), an ubiquitin promoter (e.g., Christensen et al. (1992) Plant Mol Biol 18:675-689, and Christensen & Quail (1996) Transgen Res 5:213-218), a metallothionin promoter (e.g., US2010/0064390), a Rab17 promoter (e.g., Vilardell et al. (1994) Plant Mol Biol 24:561-569), a conglycinin promoter (e.g., Chamberland et al. (1992) Plant Mol Biol 19:937-949), a plasma membrane intrinsic (PIP) promoter (e.g., Alexandersson et al. (2009) Plant J 61:650-660), a lipid transfer protein (LTP) promoter (e.g., US2009/0158464, US2009/0070893, and US2008/0295201), a gamma zein promoter (e.g., Uead et al. (1994) Mol Cell Biol 14:4350-4359), a gamma kafarin promoter (e.g., Mishra et al. (2008) Mol Biol Rep 35:81-88), a globulin promoter (e.g., Liu et al. (1998) Plant Cell Rep 17:650-655), a legumin promoter (e.g., U.S. Pat. No. 7,211,712), an early endosperm promoter (EEP) (e.g., US2007/0169226 and US2009/0227013), a B22E promoter (e.g., Klemsdal et al. (1991) Mol Gen Genet 228:9-16), an oleosin promoter (e.g., Plant et al. (1994) Plant Mol Biol 25:193-205), an early abundant protein (EAP) promoter (e.g., U.S. Pat. No. 7,321,031), a late embryogenesis abundant (LEA) protein (e.g., Hval, Straub et al. (1994) Plant Mol Biol 26:617-630; Dhn and WSI18, Xiao & Xue (2001) Plant Cell Rep 20:667-673), In2-2 promoter (De Veylder et al. (1997) Plant Cell Physiol 38:568-577), a glutathione S-transferase (GST) promoter (e.g., WO93/01294), a PR promoter (e.g., Cao et al. (2006) Plant Cell Rep 6:554-560, and Ono et al. (2004) Biosci Biotech Biochem 68:803-807), an ACE1 promoter (e.g., Mett et al. (1993) Proc Natl Acad Sci USA 90:4567-4571), a steroid responsive promoter (e.g., Schena et al. (1991) Proc Natl Acad Sci USA 88:10421-10425, and McNellis et al. (1998) Plant J 14:247-257), an ethanol-inducible promoter (e.g., AlcA, Caddick et al. (1988) Nat Biotechnol 16:177-180), an estradiol-inducible promoter (e.g., Bruce et al. (2000) Plant Cell 12:65-79), an XVE estradiol-inducible promoter (e.g., Zao et al. (2000) Plant J 24: 265-273), a VGE methoxyfenozide-inducible promoter (e.g., Padidam et al. (2003) Transgen Res 12:101-109), or a TGV dexamethasone-inducible promoter (e.g., Bohner et al. (1999) Plant J 19:87-95).

i. Ligand Responsive Promoters

As used herein, a “ligand responsive promoter” comprises a minimal promoter sequence and at least one operator sequence which is capable of activating transcription of an operably linked polynucleotide. A minimal promoter sequence, as used herein, comprises at least the minimal number of regulatory elements which are needed to direct a basal level of transcription. Such promoters can further include any number of additional elements, such as, operator sequences, enhancers or other transcriptional regulatory elements which influence transcription levels in a desired manner. Such a ligand responsive promoter can be used in combination with the various SuR and revSuRs discussed herein to aid in the controlled expression of a sequence of interest. It is understood that depending on the minimal promoter sequence employed with the ligand responsive elements, a promoter can be designed to produce varying levels of transcriptional activity in the absence of the ligand-dependent transcriptional regulator.

For example, when employing a revSuR linked to a transcriptional activation domain (revSuR-TAD), in the presence of an effective concentration of SU ligand, the revSuR-TAD can bind one or more of the operators of the ligand responsive promoter and increase transcription of the operably linked sequence of interest. In the absence of an effective amount of the SU ligand, the revSuR-TA can no longer bind the operator and the operably linked polynucleotide is transcribed at the base level of the minimal promoter.

In other embodiments, in the absence of an effective concentration of SU ligand, an SuR that is linked to a transcriptional repression domain (SuR-TR; similar to that of TetR in U.S. Pat. No. 6,271,348, which is herein incorporated by reference in its entirety) can bind one or more operators of the ligand responsive promoter and further minimize basal transcription. In the presence of an effective concentration of the SU ligand, the SuR can no longer bind the operator and transcription of the operably linked polynucleotide is de-repressed.

Any combination of promoters and operators may be employed to form a ligand responsive promoter. Operators of interest include, but are not limited to, a TetR(A) operator sequence, a TetR(B) operator sequence, a TetR(D) operator sequence, TetR(E) operator sequence, a TetR(H) operator sequence, or an active variant or fragment thereof. Additional operators of interest include, but are not limited to, those that are regulated by the following repressors: tet, lac, trp, phd, arg, LexA, phiCh1 repressor, lambda C1 and Cro repressors, phage X repressor, MetJ, phir1t rro, phi434 C1 and Cro repressors, RafR, gal, ebg, uxuR, exuR, ROS, SinR, PurR, FruR, P22 C2, TetC, AcrR, Bet1, Bm3R1, EnvR, QacR, MtrR, TcmR, Ttk, YbiH, YhgD, and mu Ner, or DNA binding domains in Interpro families including but not limited to IPR001647, IPR010982, and IPR011991.

In one embodiment, the promoter is a minimal promoter with the sole intention of activating transcription beyond its minimal state.

In a second embodiment, the promoter is a repressible promoter whereby the promoter maintains all normal characteristics of the promoter i.e. constitutive, tissue specific, temporal specific etc., yet due to strategically embedded operator sequences can be conditionally repressed by SuR. In a further refinement of this technology the SuR can be translationally fused to a transcription repression domain (analogous to that of TetR in U.S. Pat. No. 6,271,348) and thus block access of the transcription complex both directly thru binding to operator sequences and indirectly thru heterochromatin formation following recruitment of the histone deacetylase complex.

In a third embodiment, the promoter can be a hybrid promoter whose transcription is both conditionally repressed and activated based on the presence/absence of sulfonylurea and SU responsive repressors and activators. In this example, operators are juxtaposed to the TATA box and/or transcriptional start site to enable active repression thru binding of SuR in the absence of SU while additional operators are located upstream of the TATA box or downstream of the transcriptional start site as a landing pad to enable transcriptional activation by revSuR-TA in the presence of SU. In this example, the operators targeted for repression would only be recognized by the SuR in the absence of ligand while the operators located upstream of the promoters would be bound by the revSuR-TAD activator in the presence of ligand. In a further refinement of this technology the SuR could be a hybrid protein with a transcriptional repression domain i.e. SuR-TR. See, for example Berens and Hillens (2003) Eur. J. Biochem. 207:1309-3121, herein incorporated by reference in its entirety.

In one embodiment, the ligand responsive promoter comprises at least one tet operator sequence. Binding of a sulfonylurea-responsive regulator to a tet operator is controlled by sulfonylurea compounds and analogs thereof. The tet operator sequence can be located within 0-30 nucleotides 5′ or 3′ of the TATA box of the ligand responsive promoter, including, for example, within 20, 19, 18, 17, 16, 15, 14, 13, 12, 11, 10, 9, 8, 7, 6, 5, 4, 3, 2, 1, or 0 nt of the TATA box. In other instances, the tet operator sequence may partially overlap with the TATA box sequence. In one non-limiting example, the tet operator sequence is SEQ ID NO:848 or an active variant or fragment thereof.

Useful tet operator containing promoters include, for example, those known in the art (see, e.g., Matzke et al. (2003) Plant Mol Biol Rep 21:9-19; Padidam (2003) Curr Op Plant Biol 6:169-177; Gatz & Quail (1988) PNAS 85:1394-1397; Ulmasov et al. (1997) Plant Mol Biol 35:417-424; Weinmann et al. (1994) Plant J 5:559-569). One or more tet operator sequences can be added to a promoter in order to produce a tetracycline inducible promoter. See, for example, Weinmann et al. (1994) Plant J 5:559-569; Love et al. (2000) Plant J 21:579-588. In addition, a widely tested tetracycline regulated expression system for plants using the CaMV 35S promoter was developed (Gatz et al. (1992) Plant J 2:397-404) having three tet operators introduced near the TATA box (3×OpT 35S).

Thus, a ligand responsive promoter comprising at least one, two, three or more operators (including a tet operator, such as that set forth in SEQ ID NO:848 or an active variant or fragment thereof) regulating expression of said repressor can be used. Non-limiting ligand responsive promoters for expression of the chemically-regulated transcriptional repressor, include the ligand responsive promoters set forth in SEQ ID NO:885, 856, 857, 858, 859, or 860 or active variants and fragments thereof.

Any promoter can be combined with an operator to generate a ligand responsive promoter. In specific embodiments, the promoter is active in plant cells. The promoter can be a constitutive promoter or a non-constitutive promoter. Non-constitutive promoters include tissue-preferred promoter, such as a promoter that is primarily expressed in roots, leaves, stems, flowers, silks, anthers, pollen, meristem, seed, endosperm, or embryos.

In particular embodiments, the promoter is a plant actin promoter, a banana streak virus promoter (BSV), an MMV promoter, an enhanced MMV promoter (dMMV), a plant P450 promoter, or an elongation factor 1a (EF1A) promoter. Promoters of interest include, for example, a plant actin promoter (SEQ ID NO:849), a banana streak virus promoter (BSV) (SEQ ID NO:850), a mirabilis mosaic virus promoter (MMV) (SEQ ID NO:851), an enhanced MMV promoter (dMMV) (SEQ ID NO:852), a plant P450 promoter (MP1) (SEQ ID NO:853), or an elongation factor 1a (EF1A) promoter (SEQ ID NO:854), or an active variant for fragment thereof.

The ligand responsive promoter can comprise one or more operator sequences. For example, the ligand responsive promoter can comprise 1, 2, 3, 4, 5, 6, 7, 8, 9, 10, 11, 12, 13, 14, 15 or more operator sequences. In one embodiment, the ligand responsive promoter comprises two tet operator sequences, wherein the 1^{st} tet operator sequence is located within 0-30 nt 5′ of the TATA box and the 2^{nd} tet operator sequence is located within 0-30 nt 3′ of the TATA box. In some examples, the first and/or the second tet operator sequence is located within 20, 19, 18, 17, 16, 15, 14, 13, 12, 11, 10, 9, 8, 7, 6, 5, 4, 3, 2, 1, or 0 nt of the TATA box. In some examples the first and/or the second tet operator sequence may partially overlap with the TATA box sequence. In some examples, the first and/or the second tet operator sequence is SEQ ID NO:848 or an active variant or fragment thereof.

In other embodiments, the ligand responsive promoter comprises three tet operator sequences, wherein the 1^{st} tet operator sequence is located within 0-30 nt 5′ of the TATA box, and the 2^{nd} tet operator sequence is located within 0-30 nt 3′ of the TATA box, and the 3^{rd} tet operator is located with 0-50 nt of the transcriptional start site (TSS). In some examples, the 1^{st} and/or the 2^{nd} tet operator sequence is located within 20, 19, 18, 17, 16, 15, 14, 13, 12, 11, 10, 9, 8, 7, 6, 5, 4, 3, 2, 1, or 0 nt of the TATA box. In other instances, the 3^{rd} tet operator sequence is located within 50, 45, 40, 35, 30, 25, 20, 19, 18, 17, 16, 15, 14, 13, 12, 11, 10, 9, 8, 7, 6, 5, 4, 3, 2, 1, or 0 nt of the TSS. In some examples, the 3^{rd} tet operator is located 5′ of the TSS, or the 3^{rd} tet operator sequence may partially overlap with the TSS sequence. In one non-limiting embodiment, the 1^{st}, 2^{nd} and/or the 3^{rd} tet operator sequence is SEQ ID NO:848 or active variant or fragment thereof.

In specific examples, the ligand responsive promoter is a plant actin promoter (actin/Op) (SEQ ID NO:855), a banana streak virus promoter (BSV/Op) (SEQ ID NO:856), a mirabilis mosaic virus promoter (MMV/Op) (SEQ ID NO:857), an enhanced MMV promoter (dMMV/Op) (SEQ ID NO:858), a plant P450 promoter (MP1/Op) (SEQ ID NO:859), or an elongation factor 1a (EF1A/Op) promoter (SEQ ID NO:860) or an active variant or fragment thereof. Thus, the ligand responsive promoter can comprise a polynucleotide sequence having at least about 50%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO:885, 856, 857, 858, 859, or 860, wherein the promoter retains ligand responsive promoter activity. In a specific example, the promoter comprises a polynucleotide sequence having at least 95% sequence identity to SEQ ID NO:885, 856, 857, 858, 859, or 860, wherein the promoter retains ligand responsive promoter activity.

In some embodiments, the ligand responsive promoter employed in the chemical-gene switch or to express the fusion protein comprising the SU-dependent stabilization domain is expressed in various tissues or cells, restricted to selected tissue or cell type, restricted to specific developmental stage(s), restricted to specific environmental conditions, and/or restricted to specific generation of a plant or progeny thereof. In some examples, the polynucleotide of interest operably linked to a ligand responsive promoter that, when un-repressed, expresses primarily expressed in roots, leaves, stems, flowers, silks, anthers, pollen, meristem, germline, seed, endosperm, embryos, or progeny. In some examples, expression of the polynucleotide of interest or the fusion protein comprising the SU-dependent stabilization domain operably linked to the ligand responsive promoter results in expression occurring primarily at specific times, which include but are not limited to seed or plant developmental stages, vegetative growth, reproductive cycle, response to environmental conditions, response to pest or pathogen presence, response to chemical compounds, or any combination thereof. In other embodiments, expression of the polynucleotide of interest or the fusion protein comprising the SU-dependent stabilization domain is reduced, inhibited, or blocked in various tissues or cells, which may be restricted to selected tissue or cell type, restricted to specific developmental stage(s), restricted to specific environmental conditions, and/or restricted to specific generation of a plant or progeny thereof. In some examples expression of the polynucleotide of interest or the fusion protein comprising the SU-dependent stabilization domain is primarily inhibited in roots, leaves, stems, flowers, silks, anthers, pollen, meristem, germline, seed, endosperm, embryos, or progeny. In some examples expression of the polynucleotide of interest occurs primarily inhibited at specific times, which include but are not limited to seed or plant developmental stages, vegetative growth, reproductive cycle, response to environmental conditions, response to pest or pathogen presence, response to chemical compounds, or any combination thereof.

### VII. Polynucleotide Constructs

The use of the term “polynucleotide” is not intended to limit the methods and compositions to polynucleotides comprising DNA. Those of ordinary skill in the art will recognize that polynucleotides can comprise ribonucleotides and combinations of ribonucleotides and deoxyribonucleotides. Such deoxyribonucleotides and ribonucleotides include both naturally occurring molecules and synthetic analogues. The polynucleotides of the invention also encompass all forms of sequences including, but not limited to, single-stranded forms, double-stranded forms, hairpins, stem-and-loop structures, and the like.

The various polynucleotide sequences employed herein can be provided in expression cassettes for expression in the host cell or plant of interest. The cassette can include 5′ and 3′ regulatory sequences operably linked to the chemically-regulated transcriptional repressor, the silencing element and the polynucleotide of interest. “Operably linked” is intended to mean a functional linkage between two or more elements. For example, an operable linkage between a polynucleotide of interest and a regulatory sequence (i.e., a promoter) is a functional link that allows for expression of the polynucleotide of interest. Operably linked elements may be contiguous or non-contiguous. When used to refer to the joining of two protein coding regions, by operably linked is intended that the coding regions are in the same reading frame. The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of the polynucleotide to be under the transcriptional regulation of the regulatory regions. The expression cassette may additionally contain selectable marker genes.

The expression cassette can include in the 5′-3′ direction of transcription, a transcriptional and translational initiation region (i.e., a promoter), a polynucleotide disclosed herein, and a transcriptional and translational termination region (i.e., termination region) functional in the host cell or plant. The regulatory regions (i.e., promoters, transcriptional regulatory regions, and translational termination regions) and/or the various polynucleotides operably linked to the promoter may be native/analogous to the host cell or to each other. Alternatively, the regulatory regions may be heterologous to the host cell or to each other.

As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide.

The termination region may be native with the transcriptional initiation region, may be native with the plant host, or may be derived from another source (i.e., foreign or heterologous) to the promoter, the plant host, or any combination thereof. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

Where appropriate, the various polynucleotides disclosed herein may be optimized for increased expression in the transformed plant. That is, the polynucleotides can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385. See also, Della-Cioppa et al. (1987) Plant Physiol. 84:965-968.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

As discussed in detail elsewhere herein, a number of promoters can be used to express the various components. The promoters can be selected based on the desired outcome.

The expression cassette(s) can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glyphosate, glufosinate ammonium, bromoxynil, sulfonylureas, dicamba, and 2,4-dichlorophenoxyacetate (2,4-D). Additional selectable markers include phenotypic markers such as β-galactosidase and fluorescent proteins such as green fluorescent protein (GFP) (Su et al. (2004) Biotechnol Bioeng 85:610-9 and Fetter et al. (2004) Plant Cell 16:215-28), cyan florescent protein (CYP) (Bolte et al. (2004) J Cell Science 117:943-54 and Kato et al. (2002) Plant Physiol 129:913-42), and yellow florescent protein (PhiYFP™ from Evrogen, see, Bolte et al. (2004) J. Cell Science 117:943-54). For additional selectable markers, see generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992)Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Aci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference. The above list of selectable marker genes is not meant to be limiting.

The various components can be introduced into a host cell or plant on a single polynucleotide construct or single plasmid or on separate polynucleotide constructs or on separate plasmids. It is further recognized the various components disclosed herein can be brought together through any means including the introduction of one or more component into a plant and then breeding the individual components together into a single plant.

### IIX. Host Cells

The various DNA constructs disclosed herein can be introduced/expressed in a host cell such as bacteria, yeast, insect, mammalian, or plant cells. It is expected that those of skill in the art are knowledgeable in the numerous systems available for the introduction of a polypeptide or a nucleotide sequence of the present invention into a host cell. No attempt to describe in detail the various methods known for providing proteins in prokaryotes or eukaryotes will be made.

By “host cell” is meant a cell, which comprises a heterologous nucleic acid sequence of the invention. Host cells may be prokaryotic cells such as E. coli, or eukaryotic cells such as yeast, insect, amphibian, or mammalian cells. Host cells can also be monocotyledonous or dicotyledonous plant cells. In one embodiment, the monocotyledonous host cell is a maize host cell.

Plants, plant cells, plant parts and seeds, and grain having one or more of the recombinant constructs disclosed herein are provided. In specific embodiments, the plants and/or plant parts have stably incorporated at least one of the recombinant constructs.

As used herein, the term plant includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides.

Various plant species that can comprise a host cell include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, grasses and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tuhpa spp.), daffodils (Narcissus spp.), petunias (Petunia hybrida), carnation (Dianthus caryophyllus), poinsettia (Euphorbia pulcherrima), and chrysanthemum.

Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). In specific embodiments, plants of the present invention are crop plants (for example, corn, alfalfa, sunflower, Brassica, soybean, cotton, safflower, peanut, sorghum, wheat, millet, tobacco, etc.). In other embodiments, corn and soybean plants are optimal, and in yet other embodiments corn plants are optimal.

Other plants of interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

A “subject plant or plant cell” is one in which genetic alteration, such as transformation, has been affected as to a gene of interest, or is a plant or plant cell which is descended from a plant or cell so altered and which comprises the alteration. A “control” or “control plant” or “control plant cell” provides a reference point for measuring changes in phenotype of the subject plant or plant cell.

A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e. with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the gene of interest and/or the silencing element; (e) the subject plant or plant cell itself, under conditions in which the gene of interest is not expressed.

As outlined above, plants and plant parts having any one of the recombinant constructs disclosed herein can further display tolerance to the SU chemical ligand. The tolerance to the SU ligand can be naturally occurring or can be generated by human intervention via breeding or the introduction of recombination sequences that confer tolerance to the SU ligand. Thus, in some instances the plants comprising the chemical-gene switch comprise sequence that confer tolerant to a SU herbicide, including for example altered forms of AHAS, including the HRA sequence.

### IX. Introducing Polynucleotides

The methods provided herein comprise introducing a polypeptide or polynucleotide into a host cell (i.e., a plant). “Introducing” is intended to mean presenting to the host cell (i.e., a plant cell) the polynucleotide or polypeptide in such a manner that the sequence gains access to the interior of a cell. The methods of the invention do not depend on a particular method for introducing a sequence into the host cell, only that the polynucleotide or polypeptides gains access to the interior of at least one cell of the host. Methods for introducing polynucleotide or polypeptides into host cells (i.e., plants) are known in the art and include, but are not limited to, stable transformation methods, transient transformation methods, and virus-mediated methods.

“Stable transformation” is intended to mean that the nucleotide construct introduced into a host (i.e., a plant) integrates into the genome of the plant and is capable of being inherited by the progeny thereof “Transient transformation” is intended to mean that a polynucleotide is introduced into the host (i.e., a plant) and expressed temporally or a polypeptide is introduced into a host (i.e., a plant).

Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 4:320-334), electroporation (Riggs et al. (1986) Proc. Natl. Acad. Sci. USA 83:5602-5606, Agrobacterium-mediated transformation (Townsend et al., U.S. Pat. No. 5,563,055; Zhao et al., U.S. Pat. No. 5,981,840), direct gene transfer (Paszkowski et al. (1984) EMBO J. 3:2717-2722), and ballistic particle acceleration (see, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

In specific embodiments, the various constructs disclosed herein can be provided to a host cell (i.e., a plant cell) using a variety of transient transformation methods. Such methods include, for example, microinjection or particle bombardment. See, for example, Crossway et al. (1986) Mol Gen. Genet. 202:179-185; Nomura et al. (1986) Plant Sci. 44:53-58; Hepler et al. (1994) Proc. Natl. Acad. Sci. 91: 2176-2180 and Hush et al. (1994) The Journal of Cell Science 107:775-784, all of which are herein incorporated by reference. Alternatively, the various polynucleotides can be transiently transformed into the host cell (i.e., a plant cell) using techniques known in the art. Such techniques include viral vector system and the precipitation of the polynucleotide in a manner that precludes subsequent release of the DNA. Thus, the transcription from the particle-bound DNA can occur, but the frequency with which it is released to become integrated into the genome is greatly reduced. Such methods include the use particles coated with polyethylimine (PEI; Sigma #P3143).

In other embodiments, the polynucleotides disclosed herein may be introduced into the host cells (i.e., a plant cell) by contacting the host cell with a virus or viral nucleic acids. Generally, such methods involve incorporating a nucleotide construct of the invention within a viral DNA or RNA molecule. Further, it is recognized that promoters employed can also encompass promoters utilized for transcription by viral RNA polymerases. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931, and Porta et al. (1996) Molecular Biotechnology 5:209-221; herein incorporated by reference.

Methods are known in the art for the targeted insertion of a polynucleotide at a specific location in the plant genome. In one embodiment, the insertion of the polynucleotide at a desired genomic location is achieved using a site-specific recombination system. See, for example, WO99/25821, WO99/25854, WO99/25840, WO99/25855, and WO99/25853, all of which are herein incorporated by reference.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting progeny having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having at least one recombinant polynucleotide disclosed herein, stably incorporated into their genome.

In some examples, the various recombinant polynucleotides can be introduced into a plastid, either by transformation of the plastid or by directing a transcript or polypeptide into the plastid. Any method of transformation, nuclear or plastid, can be used, depending on the desired product and/or use. Plastid transformation provides advantages including high transgene expression, control of transgene expression, ability to express polycistronic messages, site-specific integration via homologous recombination, absence of transgene silencing and position effects, control of transgene transmission via uniparental plastid gene inheritance and sequestration of expressed polypeptides in the organelle which can obviate possible adverse impacts on cytoplasmic components (e.g., see, reviews including Heifetz (2000) Biochimie 82:655-666; Daniell et al. (2002) Trends Plant Sci 7:84-91; Maliga (2002) Curr Op Plant Biol 5:164-172; Maliga (2004) Ann Rev Plant Biol 55-289-313; Daniell et al. (2005) Trends Biotechnol 23:238-245 and Verma and Daniell (2007) Plant Physiol 145:1129-1143).

Methods and compositions of plastid transformation are well known, for example, transformation methods include (Boynton et al. (1988) Science 240:1534-1538; Svab et al. (1990) Proc Natl Acad Sci USA 87:8526-8530; Svab et al. (1990) Plant Mol Biol 14:197-205; Svab et al. (1993) Proc Natl Acad Sci USA 90:913-917; Golds et al. (1993) Bio/Technology 11:95-97; O'Neill et al. (1993) Plant J 3:729-738; Koop et al. (1996) Planta 199:193-201; Kofer et al. (1998) In Vitro Plant 34:303-309; Knoblauch et al. (1999) Nat Biotechnol 17:906-909); as well as plastid transformation vectors, elements, and selection (Newman et al. (1990) Genetics 126:875-888; Goldschmidt-Clermont, (1991) Nucl Acids Res 19:4083-4089; Carrer et al. (1993) Mol Gen Genet 241:49-56; Svab et al. (1993) Proc Natl Acad Sci USA 90:913-917; Verma and Daniell (2007) Plant Physiol 145:1129-1143).

Methods and compositions for controlling gene expression in plastids are well known including (McBride et al. (1994) Proc Natl Acad Sci USA 91:7301-7305; Lössel et al. (2005) Plant Cell Physiol 46:1462-1471; Heifetz (2000) Biochemie 82:655-666; Surzycki et al. (2007) Proc Natl Acad Sci USA 104:17548-17553; U.S. Pat. Nos. 5,576,198 and 5,925,806; WO 2005/0544478), as well as methods and compositions to import polynucleotides and/or polypeptides into a plastid, including translational fusion to a transit peptide (e.g., Comai et al. (1988) J Biol Chem 263:15104-15109).

A variety of eukaryotic expression systems or prokaryotic expression systems such as bacterial, yeast, insect cell lines, plant and mammalian cells, are known to those of skill in the art. As explained briefly below, a recombinant polynucleotide disclosed herein can be expressed in these eukaryotic systems.

Synthesis of heterologous polynucleotides in yeast is well known (Sherman et al. (1982) Methods in Yeast Genetics, Cold Spring Harbor Laboratory). Two widely utilized yeasts for production of eukaryotic proteins are Saccharomyces cerevisiae and Pichia pastoris. Vectors, strains, and protocols for expression in Saccharomyces and Pichia are known in the art and available from commercial suppliers (e.g., Invitrogen). Suitable vectors usually have expression control sequences, such as promoters, including 3-phosphoglycerate kinase or alcohol oxidase, and an origin of replication, termination sequences and the like as desired.

A protein of the present invention, once expressed, can be isolated from yeast by lysing the cells and applying standard protein isolation techniques to the lists. The monitoring of the purification process can be accomplished by using Western blot techniques or radioimmunoassay of other standard immunoassay techniques.

The various recombinant sequences disclosed herein can also be ligated to various expression vectors for use in transfecting cell cultures of, for instance, mammalian, insect, or plant origin. Illustrative cell cultures useful for the production of the peptides are mammalian cells. A number of suitable host cell lines capable of expressing intact proteins have been developed in the art, and include the HEK293, BHK21, and CHO cell lines. Expression vectors for these cells can include expression control sequences, such as an origin of replication, a promoter (e.g. the CMV promoter, a HSV tk promoter or pgk (phosphoglycerate kinase) promoter), an enhancer (Queen et al. (1986) Immunol. Rev. 89:49), and necessary processing information sites, such as ribosome binding sites, RNA splice sites, polyadenylation sites (e.g., an SV40 large T Ag poly A addition site), and transcriptional terminator sequences. Other animal cells useful for production of proteins of the present invention are available, for instance, from the American Type Culture Collection.

Appropriate vectors for expressing the recombinant sequences disclosed herein in insect cells are usually derived from the SF9 baculovirus. Suitable insect cell lines include mosquito larvae, silkworm, armyworm, moth and Drosophila cell lines such as a Schneider cell line (See, Schneider (1987) J. Embryol. Exp. Morphol. 27:353-365).

As with yeast, when higher animal or plant host cells are employed, polyadenylation or transcription terminator sequences are typically incorporated into the vector. An example of a terminator sequence is the polyadenylation sequence from the bovine growth hormone gene. Sequences for accurate splicing of the transcript may also be included. An example of a splicing sequence is the VP1 intron from SV40 (Sprague et al. (1983) J Virol. 45:773-781). Additionally, gene sequences to control replication in the host cell may be incorporated into the vector such as those found in bovine papilloma virus type-vectors (Saveria-Campo (1985) DNA Cloning Vol. II a Practical Approach, D. M. Glover, Ed., IRL Press, Arlington, Va., pp. 213-238).

Animal and lower eukaryotic (e.g., yeast) host cells are competent or rendered competent for transfection by various means. There are several well-known methods of introducing DNA into animal cells. These include: calcium phosphate precipitation, fusion of the recipient cells with bacterial protoplasts containing the DNA, treatment of the recipient cells with liposomes containing the DNA, DEAE dextrin, electroporation, biolistics, and micro-injection of the DNA directly into the cells. The transfected cells are cultured by means well known in the art (Kuchler (1997) Biochemical Methods in Cell Culture and Virology, Dowden, Hutchinson and Ross, Inc.).

### X. Methods of Use

The various SU-dependent stabilization domains described herein, can be used in a variety of different methods to influence the level of a sequence of interest.

i. Methods of Using the Fusion Protein Comprising the SU-Dependent Stabilization Domain

In one embodiment, a method to modulate the stability of a polypeptide of interest in a cell is provided. The method comprises (a) providing a cell having a recombinant polynucleotide comprising a nucleotide sequence encoding a polypeptide having a SU-dependent stabilization domain operably linked to a polynucleotide encoding the polypeptide of interest; (b) expressing the recombinant polynucleotide in the cell; and, (c) contacting the cell with an effective amount of a SU ligand, wherein the effective amount of the SU ligand increases the level the polypeptide of interest in the cell. This method has the advantages of reducing genetic complexity to one expression cassette instead of two cassettes which are often required for transcriptional regulation (i.e., one for the target gene and one for the transcriptional activator/repressor) and, in some instance, this method could enable a quicker response to ligand as both transcription and translation would have already reached steady state. The promoter driving expression of the destabilized protein could be constitutive, spatio-temporal specific, or inducible. Accumulation of the target gene product in any cell type would be dependent on the presence of the stabilizing ligand.

In some embodiments, the SU-dependent stabilization domain comprises (a) a ligand binding domain of a SU chemically-regulated transcriptional regulator having at least one destabilization mutation; (b) a DNA binding domain of a SU chemically-regulated transcriptional regulator having at least one destabilization mutation; or (c) the SU-dependent stabilization domain comprises both (a) and (b). Various forms of such SU-dependent stabilization domains are described in further detail elsewhere herein. Such methods can further employ the use of an intein. Such constructs and how they are generated are discussed elsewhere herein.

In specific embodiments, the SU-dependent stabilization domain comprises a polypeptide having at least 80%, 85%, 90%, 91%, 92%, 93%, 94%, 95%, 96%, 97%, 98%, or 99% 100% sequence identity to the ligand binding domain of an amino acid sequence set forth in any one of SEQ ID NO:3-419, 863-870, and/or 884-889, wherein the polypeptide further comprises at least one destabilization mutation.

In further embodiments, the encoded polypeptide having the SU-dependent stabilization domain comprises a SU chemically-regulated transcriptional regulator. The SU chemically-regulated transcriptional regulator can comprise Su(R). In such instances, non-limiting examples of the SuR comprise polypeptides that share at least 80%, 85%, 90%, 91%, 92%, 93%, 94%, 95%, 96%, 97%, 98%, or 99% 100% sequence identity to any one of the polypeptides set forth any one of SEQ ID NO:3-411, 863-870, and/or 884-889, wherein said polypeptide further comprises at least one destabilization mutation.

In other embodiments, the SU chemically-regulated transcriptional regulator can comprise a revSuR. In such instances, non-limiting examples of the revSuR shares at least 80%, 85%, 90%, 91%, 92%, 93%, 94%, 95%, 96%, 97%, 98%, or 99% 100% sequence identity to any one of the polypeptides set forth any one of SEQ ID NO:412-419, wherein said polypeptide further comprises at least one destabilization mutation. When a revSuR is employed, in specific embodiments, the revSuR can further comprise a transcriptional activator domain.

In methods where the recombinant polynucleotide encodes a revSuR-TAD having at least one destabilization domain in the revSuR fused in frame to the polypeptide of interest, the recombination polynucleotide can be operably linked to any promoter, as disclosed herein, but in specific embodiments, the recombinant polynucleotide is operably linked to a promoter comprising at least one, two or three cognate operators for the encoded revSuR-TAD.

ii. Methods of Using the SU-Dependent Stabilization Domain in a Chemical-Gene Switch System

In other embodiments, methods to regulate expression in a host cell or plant are provided which employ a chemical-gene switch. Such methods comprise providing a cell (i.e., a plant cell) comprising (i) a first recombinant construct comprising a first promoter operably linked to a revSuR comprising a transcriptional activator domain, wherein the revSuR comprises a destabilization mutation; and, (ii) a second recombinant construct comprising a first ligand responsive promoter comprising at least one, two or three cognate operators for said revSuR operably linked to a polynucleotide of interest; providing the host cell (i.e, plant cell) with an effective amount of the SU ligand whereby the effective amount of the SU ligand increases the level of the revSuR-TAD and increases the level of polynucleotide of interest. In such methods, the revSuR-TAD is unstable in the absence of an effective concentration of SU ligand. The polynucleotide of interest is thereby expressed at the level of the minimal level of the ligand responsive promoter. In the presence of an effective concentration of SU ligand, the revSuR-TAD is stabilized and an increase in transcription from the ligand responsive promoter occurs.

In other methods, the destabilization mutation is found within the ligand binding domain of the revSuR; the DNA binding domain of the revSuR; or in both of the ligand binding domain and the DNA binding domain. Various forms of the revSuR and TAD that can be employed in these methods are disclosed in detail elsewhere herein.

In further embodiments, the first recombinant construct comprises a first promoter that is a ligand responsive promoter operably linked to a revSuR comprising a transcriptional activator domain, wherein the revSuR comprises a destabilization mutation. In such instances, the second ligand responsive promoter comprises at least one, two or three cognate operators for the revSuR-TAD. In still further embodiments, the cognate operator comprises the tet operator. In such embodiments, the presence of the effective concentration of SU ligand allows for an increase in expression of the revSuR-TAD.

The chemical-gene switch can thereby be employed in methods which stringently and/or specifically controlling expression of a polynucleotide of interest. Stringency and/or specificity of modulating can be influenced by selecting the combination of elements used in the switch. These include, but are not limited to each component of the chemical-gene switch. Further control is provided by selection, dosage, conditions, and/or timing of the application of the SU ligand. In some examples the expression of the polynucleotide of interest can be controlled more stringently, controlled in various tissues or cells, restricted to selected tissue or cell type, restricted to specific developmental stage(s), restricted to specific environmental conditions, and/or restricted to specific generation of a plant or progeny thereof.

In some examples, the methods and compositions comprises a chemical-gene switch which may comprise additional elements. In some examples, one or more additional elements may provide means by which expression of the polynucleotide of interest can be controlled more stringently, controlled in various tissues or cells, restricted to selected tissue or cell type, restricted to specific developmental stage(s), restricted to specific environmental conditions, and/or restricted to specific generation of a plant or progeny thereof. In some examples those elements include site-specific recombination sites, site-specific recombinases, or combinations thereof.

iii SU Ligands and Methods of Providing

Any SU ligand can be employed in the various methods disclosed herein, so long as the SU ligand is compatible with the SU-dependent stabilization domain and, when applicable, to the SuR or revSuR. A “cognate” SU ligand and SU-dependent stabilization domain are therefore compatible with one another.

A variety of SU compounds can be used as SU ligand. Sulfonylurea molecules comprise a sulfonylurea moiety (—S(O)₂NHC(O)NH(R)—). In sulfonylurea herbicides the sulfonyl end of the sulfonylurea moiety is connected either directly or by way of an oxygen atom or an optionally substituted amino or methylene group to a typically substituted cyclic or acyclic group. At the opposite end of the sulfonylurea bridge, the amino group, which may have a substituent such as methyl (R being CH₃) instead of hydrogen, is connected to a heterocyclic group, typically a symmetric pyrimidine or triazine ring, having one or two substituents such as methyl, ethyl, trifluoromethyl, methoxy, ethoxy, methylamino, dimethylamino, ethylamino and the halogens. Sulfonylurea herbicides can be in the form of the free acid or a salt. In the free acid form the sulfonamide nitrogen on the bridge is not deprotonated (i.e., —S(O)₂NHC(O)NH(R)), while in the salt form the sulfonamide nitrogen atom on the bridge is deprotonated, and a cation is present, typically of an alkali metal or alkaline earth metal, most commonly sodium or potassium. Sulfonylurea compounds include, for example, compound classes such as pyrimidinylsulfonylurea compounds, triazinylsulfonylurea compounds, thiadiazolylurea compounds, and pharmaceuticals such as antidiabetic drugs, as well as salts and other derivatives thereof. Examples of pyrimidinylsulfonylurea compounds include amidosulfuron, azimsulfuron, bensulfuron, bensulfuron-methyl, chlorimuron, chlorimuron-ethyl, cyclosulfamuron, ethoxysulfuron, flazasulfuron, flucetosulfuron, flupyrsulfuron, flupyrsulfuron-methyl, foramsulfuron, halosulfuron, halosulfuron-methyl, imazosulfuron, mesosulfuron, mesosulfuron-methyl, nicosulfuron, orthosulfamuron, oxasulfuron, primisulfuron, primisulfuron-methyl, pyrazosulfuron, pyrazosulfuron-ethyl, rimsulfuron, sulfometuron, sulfometuron-methyl, sulfosulfuron, trifloxysulfuron and salts and derivatives thereof. Examples of triazinylsulfonylurea compounds include chlorsulfuron, cinosulfuron, ethametsulfuron, ethametsulfuron-methyl, iodosulfuron, iodosulfuron-methyl, metsulfuron, metsulfuron-methyl, prosulfuron, thifensulfuron, thifensulfuron-methyl, triasulfuron, tribenuron, tribenuron-methyl, triflusulfuron, triflusulfuron-methyl, tritosulfuron and salts and derivatives thereof. Examples of thiadiazolylurea compounds include buthiuron, ethidimuron, tebuthiuron, thiazafluron, thidiazuron, pyrimidinylsulfonylurea compound (e.g., amidosulfuron, azimsulfuron, bensulfuron, chlorimuron, cyclosulfamuron, ethoxysulfuron, flazasulfuron, flucetosulfuron, flupyrsulfuron, foramsulfuron, halosulfuron, imazosulfuron, mesosulfuron, nicosulfuron, orthosulfamuron, oxasulfuron, primisulftiron, pyrazosulfuron, rimsulfuron, sulfometuron, sulfosulfuron and trifloxysulfuron); a triazinylsulfonylurea compound (e.g., chlorsulfuron, cinosulfuron, ethametsulfuron, iodosulfuron, metsulfuron, prosulfuron, thifensulfuron, triasulfuron, tribenuron, triflusulfuron and tritosulfuron); or a thiadazolylurea compound (e.g., cloransulam, diclosulam, florasulam, flumetsulam, metosulam, and penoxsulam) and salts and derivatives thereof. Examples of antidiabetic drugs include acetohexamide, chlorpropamide, tolbutamide, tolazamide, glipizide, gliclazide, glibenclamide (glyburide), gliquidone, glimepiride and salts and derivatives thereof. In some systems, the SuR polypeptides specifically bind to more than one sulfonylurea compound, so one can chose which SU ligand to apply to the plant.

In some examples, the sulfonylurea compound is selected from the group consisting of chlorsulfuron, ethametsulfuron-methyl, metsulfuron-methyl, thifensulfuron-methyl, sulfometuron-methyl, tribenuron-methyl, chlorimuron-ethyl, nicosulfuron, and rimsulfuron.

In other embodiments, the sulfonylurea compound comprises a pyrimidinylsulfonylurea, a triazinylsulfonylurea, a thiadazolylurea, a chlorosulfuron, an ethametsulfuron, a thifensulfuron, a metsulfuron, a sulfometuron, a tribenuron, a chlorimuron, a nicosulfuron, or a rimsulfuron compound.

In one embodiment, the ligand for the SU-dependent stabilization domain is ethametsulfuron. In some examples the ethametsulfuron is provided at a concentration of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 18, 19, 20, 25, 30, 35, 40, 45, 50, 55, 60, 65, 70, 75, 80, 85, 90, 100, 200 or 500 μg/ml or greater. In other examples, the ethametsulfuron is provided at a concentration of about at least 0.1, 0.5, 1, 1.5, 1.75, 2, 2.25, 2.5, 2.75, 3, 3.25, 3.5, 3.75, 4, 4.5, 5, 5.5, 6, 6.5, 7, 7.5, 8, 8.5, 9, 9.5, 10 or greater times the registered recommended rate for any particular crop. In yet other examples, the ethametsulfruon is provided at least about 0.5, 1, 2, 3, 4, 4, 5, 7, 8, 9, 10, 11, 12, 13, 14, 15, 16, 17, 18, 19, 20, 21, 22, 23, 24, or 25 or greater PPM. In some examples, ethametsulfuron-dependent stabilization domain employed comprises the ligand binding domain, the DNA binding domain or the full length SU chemically-regulated transcriptional regulator, wherein the ligand binding domain comprise at least 50% 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the ligand binding domain, the DNA binding domain or the full length SU chemically-regulated transcriptional regulator of SEQ ID NO:3-419, 863-870, and/or 884-889, wherein the sequence identity is determined over the full length of the polypeptide using a global alignment method and said domain further comprises at least one destabilization mutation.

In other embodiments, the ligand for the SU-dependent stabilization domain is chlorsulfuron. In some examples, the chlorsulfuron is provided at a concentration of about 0.01, 0.02, 0.03, 0.04, 0.05, 0.06, 0.07, 0.08, 0.09, 0.10, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 18, 19, 20, 25, 30, 35, 40, 45, 50, 55, 60, 65, 70, 75, 80, 85, 90, 100, 200 or 500 μg/ml. In other examples, the chlorsulfuron is provided at a concentration of about at least 0.1, 0.5, 1, 1.5, 1.75, 2, 2.25, 2.5, 2.75, 3, 3.25, 3.5, 3.75, 4, 4.5, 5, 5.5, 6, 6.5, 7, 7.5, 8, 8.5, 9, 9.5, 10 or greater times the registered recommended rate for any particular crop. In yet other examples, the chlorsulfuron is provided at least about 0.5, 1, 2, 3, 4, 4, 5, 7, 8, 9, 10, 11, 12, 13, 14, 15, 16, 17, 18, 19, 20, 21, 22, 23, 24, or 25 or greater PPM. In some examples, chlorsulfuron-dependent stabilization domain employed comprises the ligand binding domain, the DNA binding domain or the full length SU chemically-regulated transcriptional regulator, wherein the ligand binding domain comprise at least 50% 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the ligand binding domain, the DNA binding domain or the full length SU chemically-regulated transcriptional regulator of SEQ ID NO:3-419, 863-870, 884-889, 1193-1568 and/or 1949-2110, wherein the sequence identity is determined over the full length of the polypeptide using a global alignment method and the domain further comprises at least one destabilization mutation.

By “contacting” or “providing” to the host cell, plant or plant part is intended any method whereby an effective amount of the SU ligand is exposed to the host cell, plant, plant part, tissue or organ. The SU ligand can be applied to the plant or plant part by, for example, spraying, atomizing, dusting, scattering, coating or pouring, introducing into or on the soil, introducing into irrigation water, by seed treatment or general application or dusting at the desirable time for the purpose at hand. If tissue culture is being employed, the SU ligand can be added to the culture media.

By “effective amount” of the SU ligand is intended an amount of SU ligand that is sufficient to allow for the desirable level of expression of the polynucleotide sequence of interest in a desired host cell, host tissue, plant tissue or plant part. Generally, in reference to the fusion protein comprising the SU-dependent stabilization domain, the effective amount of the SU ligand is sufficient to increase the stability, level and/or activity of the polypeptide of interest that is fused in frame to the SU-dependent stabilization domain. In reference to the use of a SU-dependent stabilization domain in the context of the chemical-gene switch, the effective amount of the SU ligand is sufficient to influence transcription as desired for the given chemical-gene switch employed. In specific embodiments, the effective amount of the SU ligand does not significantly affect the host cell, plant or crop. The effective amount may or may not be sufficient to control weeds. When desired, the expression of the polynucleotide of interest alters the phenotype and/or the genome of the host cell or plant.

The SU ligand can be contacted to the plant in combination with an adjuvant or any other agent that provides a desired agricultural effect. As used herein, an “adjuvant” is any material added to a spray solution or formulation to modify the action of an agricultural chemical or the physical properties of the spray solution. See, for example, Green and Foy (2003) “Adjuvants: Tools for Enhancing Herbicide Performance,” in Weed Biology and Management, ed. Inderjit (Kluwer Academic Publishers, The Netherlands). Adjuvants can be categorized or subclassified as activators, acidifiers, buffers, additives, adherents, antiflocculants, antifoamers, defoamers, antifreezes, attractants, basic blends, chelating agents, cleaners, colorants or dyes, compatibility agents, cosolvents, couplers, crop oil concentrates, deposition agents, detergents, dispersants, drift control agents, emulsifiers, evaporation reducers, extenders, fertilizers, foam markers, formulants, inerts, humectants, methylated seed oils, high load COCs, polymers, modified vegetable oils, penetrators, repellants, petroleum oil concentrates, preservatives, rainfast agents, retention aids, solubilizers, surfactants, spreaders, stickers, spreader stickers, synergists, thickeners, translocation aids, uv protectants, vegetable oils, water conditioners, and wetting agents.

In addition, methods of the invention can comprise the use of a herbicide or a mixture of herbicides, as well as, one or more other insecticides, fungicides, nematocides, bactericides, acaricides, growth regulators, chemosterilants, semiochemicals, repellents, attractants, pheromones, feeding stimulants or other biologically active compounds or entomopathogenic bacteria, virus, or fungi to form a multi-component mixture giving an even broader spectrum of agricultural protection.

Methods can further comprise the use of plant growth regulators such as aviglycine, N-(phenylmethyl)-1H-purin-6-amine, ethephon, epocholeone, gibberellic acid, gibberellin A₄ and A₇, harpin protein, mepiquat chloride, prohexadione calcium, prohydrojasmon, sodium nitrophenolate and trinexapac-methyl, and plant growth modifying organisms such as Bacillus cereus strain BP01.

### XI. Novel Su Chemically-Regulated Transcriptional Regulators and Compositions and Methods Employing the Same

Further provided are methods and compositions which employ novel SU chemically-regulated transcriptional regulators. Non-limiting examples of these novel polynucleotides are set forth in SEQ ID NOS: 1193-1380 and 1949-2029 or active variants and fragments thereof and the encoded polypeptides set forth in SEQ ID NOS: 1381-1568 and 2030-2110 or active variants and fragments thereof.

Fragments and variants of SU chemically-regulated transcriptional regulators polynucleotides and polypeptides are also encompassed by the present invention. By “fragment” is intended a portion of the polynucleotide or a portion of the amino acid sequence and hence protein encoded thereby. Fragments of a polynucleotide may encode protein fragments that bind to a polynucleotide comprising an operator sequence, wherein the binding is regulated by a sulfonylurea compound. Alternatively, fragments of a polynucleotide that is useful as hybridization probes generally do not encode fragment proteins retaining biological activity. Thus, fragments of a nucleotide sequence may range from at least about 20 nucleotides, about 50 nucleotides, about 100 nucleotides, and up to the full-length polynucleotide encoding the SU chemically-regulated transcriptional regulators polypeptides.

A fragment of an SU chemically-regulated transcriptional regulators polynucleotide that encodes a biologically active portion of a SU chemically-regulated transcriptional regulator will encode at least 50, 75, 100, 150, 175, 200, 225, 250, 275, 300, 325, 350, 375, 400, 410, 415, 420, 425, 430, 435, or 440 contiguous amino acids, or up to the total number of amino acids present in a full-length SU chemically-regulated transcriptional regulators polypeptide. Fragments of an SU chemically-regulated transcriptional regulator polynucleotide that are useful as hybridization probes or PCR primers generally need not encode a biologically active portion of an SU chemically-regulated transcriptional regulator protein.

Thus, a fragment of an SU chemically-regulated transcriptional regulator polynucleotide may encode a biologically active portion of an SU chemically-regulated transcriptional regulator polypeptide, or it may be a fragment that can be used as a hybridization probe or PCR primer using methods disclosed below. A biologically active portion of an SU chemically-regulated transcriptional regulator polypeptide can be prepared by isolating a portion of one of the SU chemically-regulated transcriptional regulator polynucleotides, expressing the encoded portion of the SU chemically-regulated transcriptional regulator polypeptides (e.g., by recombinant expression in vitro), and assessing the activity of the portion of the SU chemically-regulated transcriptional regulator protein. Polynucleotides that are fragments of an SU chemically-regulated transcriptional regulator nucleotide sequence comprise at least 16, 20, 50, 75, 100, 150, 200, 250, 300, 350, 400, 450, 500, 550, 600, 650, 700, 800, 900, 1,000, 1,100, 1,200, 1,300, or 1,400 contiguous nucleotides, or up to the number of nucleotides present in a full-length SU chemically-regulated transcriptional regulator polynucleotide disclosed herein.

“Variant” protein is intended to mean a protein derived from the protein by deletion (i.e., truncation at the 5′ and/or 3′ end) and/or a deletion or addition of one or more amino acids at one or more internal sites in the native protein and/or substitution of one or more amino acids at one or more sites in the native protein. Variant proteins encompassed are biologically active, that is they continue to possess the desired biological activity of the native protein, that is, bind to a polynucleotide comprising an operator sequence, wherein the binding is regulated by a sulfonylurea compound. Such variants may result from, for example, genetic polymorphism or from human manipulation.

“Variants” is intended to mean substantially similar sequences. For polynucleotides, a variant comprises a polynucleotide having a deletion (i.e., truncations) at the 5′ and/or 3′ end and/or a deletion and/or addition of one or more nucleotides at one or more internal sites within the native polynucleotide and/or a substitution of one or more nucleotides at one or more sites in the native polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of one of the SU chemically-regulated transcriptional regulator polypeptides. Naturally occurring variants such as these can be identified with the use of well-known molecular biology techniques, as, for example, with polymerase chain reaction (PCR) and hybridization techniques as outlined below. Variant polynucleotides also include synthetically derived polynucleotides, such as those generated, for example, by using site-directed mutagenesis or gene synthesis but which still encode an SU chemically-regulated transcriptional regulator polypeptide.

Biologically active variants of an SU chemically-regulated transcriptional regulator polypeptide (and the polynucleotide encoding the same) will have at least about 75%, 80%, 85%, 90%, 91%, 92%, 93%, 94%, 95%, 96%, 97%, 98%, 99%, or more sequence identity to the polypeptide of any one of SEQ ID NO: 1381-1568 and 2030-2110 or with regard to any of the SU chemically-regulated transcriptional regulator polypeptides as determined by sequence alignment programs and parameters described elsewhere herein.

In still further embodiments, a biologically active variant of an SU chemically-regulated transcriptional regulator protein may differ from that protein by 200, 190, 180, 170, 160, 150, 140, 130, 120, 110, 100, 90, 80, 70, 60, 50, 40, 30, 25, 20, 19, 18, 17, 16 amino acid residues, as few as 1-15 amino acid residues, as few as 1-10, such as 6-10, as few as 10, 9, 8, 7, 6, 5, as few as 4, 3, 2, or even 1 amino acid residue.

The SU chemically-regulated transcriptional regulator polypeptide and the active variants and fragments thereof may be altered in various ways including amino acid substitutions, deletions, truncations, and insertions. Methods for such manipulations are generally known in the art. For example, amino acid sequence variants and fragments of the HPPD proteins can be prepared by mutations in the DNA. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

Obviously, the mutations that will be made in the DNA encoding the variant must not place the sequence out of reading frame and optimally will not create complementary regions that could produce secondary mRNA structure. See, EP Patent Application Publication No. 75,444.

Variant polynucleotides and proteins also encompass sequences and proteins derived from a mutagenic and recombinogenic procedure such as DNA shuffling. With such a procedure, one or more different SU chemically-regulated transcriptional regulator coding sequences can be manipulated to create a new SU chemically-regulated transcriptional regulator possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the SU chemically-regulated transcriptional regulator sequences disclosed herein and other known SU chemically-regulated transcriptional regulator genes to obtain a new gene coding for a protein with an improved property of interest. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

Polynucleotides encoding the SU chemically-regulated transcriptional regulator polypeptide and the active variants and fragments thereof can be introduced into any of the DNA constructs discussed herein and further can be operably linked to any promoter sequence of interest. These constructs can be introduced/expressed in a host cell such as bacteria, yeast, insect, mammalian, or plant cells. Details for such methods are disclosed elsewherein herein, as is a detailed list of plants and plant cells that the sequences can be introduced into. Thus, various host cells, plants and plant cells are provided comprising the novel SU chemically-regulated transcriptional activators, including but not limited to, monocots and dicot plants such as corn, alfalfa, sunflower, Brassica, soybean, cotton, safflower, peanut, sorghum, wheat, millet, tobacco, etc.

In one embodiment, the novel SuR can be designed to contain a variety of different DNA binding domains and thereby bind a variety of different operators and influence transcription. In one embodiment, the SuR polypeptide comprises a DNA binding domain that specifically binds to a tetracycline operator. Thus, in specific embodiments, the SuR polypeptide or the polynucleotide encoding the same can comprise a DNA binding domain, including but not limited to, an operator DNA binding domain from repressors included tet, lac, trp, phd, arg, LexA, phiCh1 repressor, lambda C1 and Cro repressors, phage X repressor, MetJ, phir1t rro, phi434 C1 and Cro repressors, RafR, gal, ebg, uxuR, exuR, ROS, SinR, PurR, FruR, P22 C2, TetC, AcrR, Bet1, Bm3R1, EnvR, QacR, MtrR, TcmR, Ttk, YbiH, YhgD, and mu Ner, or DNA binding domains in Interpro families including, but not limited to, IPR001647, IPR010982, and IPR01199, or an active variant or fragment thereof. Thus, the DNA binding specificity can be altered by fusing a SuR ligand binding domain to an alternate DNA binding domain. For example, the DNA binding domain from TetR class D can be fused to a SuR ligand binding domain to create SuR polypeptides that specifically bind to polynucleotides comprising a class D tetracycline operator. In some examples, a DNA binding domain variant or derivative can be used. For example, a DNA binding domain from a TetR variant that specifically recognizes a tetO-4C operator or a tetO-6C operator could be used (Helbl & Hillen (1998) J Mol Biol 276:313-318; Helbl et al. (1998) J Mol Biol 276:319-324).

In some examples, the chemically-regulated transcriptional repressor, or the polynucleotide encoding the same, includes a SuR polypeptide comprising a ligand binding domain comprising at least one amino acid substitution to a wild type tetracycline repressor protein ligand binding domain fused to a heterologous operator DNA binding domain which specifically binds to a polynucleotide comprising the operator sequence or derivative thereof, wherein repressor-operator binding is regulated by the absence or presence of a sulfonylurea compound. In specific embodiments, the heterologous operator DNA binding domain comprises a tetracycline operator sequence or active variant or fragment thereof, such that the repressor-operator binding is regulated by the absence or presence of a sulfonylurea compound.

In some examples, the SuR polypeptides, or polynucleotide encoding the same, comprise an amino acid substitution in the ligand binding domain of a wild type tetracycline repressor protein. In class B and D wild type TetR proteins, amino acid residues 6-52 represent the DNA binding domain. The remainder of the protein is involved in ligand binding and subsequent allosteric modification. For class B TetR residues 53-207 represent the ligand binding domain, while residues 53-218 comprise the ligand binding domain for the class D TetR. In some embodiments, the SuR polypeptides comprise at least one amino acid substitution in the ligand binding domain of a wild type TetR(B) protein, while in further examples, the SuR polypeptides comprise at least one amino acid substitution in the ligand binding domain of a wild type TetR(B) protein of SEQ ID NO:1.

In non-limiting embodiments, the SuR polypeptides can have an equilibrium binding constant for a sulfonylurea compound greater than 0.1 nM and less than 10 μM. In some examples, the SuR polypeptide has an equilibrium binding constant for a sulfonylurea compound of at least 0.1 nM, 0.5 nM, 1 nM, 10 nM, 50 nM, 100 nM, 250 nM, 500 nM, 750 nM, 1 μM, 5 μM, 7 μM but less than 10 μM. In other examples, the SuR polypeptide has an equilibrium binding constant for a sulfonylurea compound of at least 0.1 nM, 0.5 nM, 1 nM, 10 nM, 50 nM, 100 nM, 250 nM, 500 nM, 750 nM but less than 1 μM. In some embodiments, the SuR polypeptide has an equilibrium binding constant for a sulfonylurea compound greater than 0 nM, but less than 0.1 nM, 0.5 nM, 1 nM, 10 nM, 50 nM, 100 nM, 250 nM, 500 nM, 750 nM, 1 μM, 5 μM, 7 μM or 10 μM. In some examples, the sulfonylurea compound is a chlorsulfuron, an ethametsulfuron, a metsulfuron, a sulfometuron, a tribenuron, a chlorimuron, a nicosulfuron, a rimsulfuron and/or a thifensulfuron. In further embodiments, the SuR as set forth in SEQ ID NOS: 1381-1568 and 2030-2110 has an equilibrium binding constant for chlorsulruon. In other embodiments, the SuR as set forth in SEQ ID NO: 1381-1568 and 2030-2110 has an equilibrium binding constant for ethametsulfuron.

In some examples, the SuR polypeptides have an equilibrium binding constant for an operator sequence greater than 0.1 nM and less than 10 μM. In some examples the SuR polypeptide has an equilibrium binding constant for an operator sequence of at least 0.1 nM, 0.5 nM, 1 nM, 10 nM, 50 nM, 100 nM, 250 nM, 500 nM, 750 nM, 1 μM, 5 μM, 7 μM but less than 10 μM. In some examples, the SuR polypeptide has an equilibrium binding constant for an operator sequence of at least 0.1 nM, 0.5 nM, 1 nM, 10 nM, 50 nM, 100 nM, 250 nM, 500 nM, 750 nM but less than 1 μM. In some examples the SuR polypeptide has an equilibrium binding constant for an operator sequence greater than 0 nM, but less than 0.1 nM, 0.5 nM, 1 nM, 10 nM, 50 nM, 100 nM, 250 nM, 500 nM, 750 nM, 1 μM, 5 μM, 7 μM or 10 μM. In some examples, the operator sequence is a Tet operator sequence. In some examples, the Tet operator sequence is a TetR(A) operator sequence, a TetR(B) operator sequence, a TetR(D) operator sequence, TetR(E) operator sequence, a TetR(H) operator sequence, or a functional derivative thereof.

Various chemical ligands, including exemplary sulfonylurea chemical ligands, and the level and manner of application are discussed in detail elsewhere herein.

Various methods of employing Non-limiting examples of SuR polypeptides are set forth in U.S. Utility application Ser. No. 13/086,765, filed on Apr. 14, 2011 and in US Application Publication 2010-0105141, both of which are herein incorporated by reference in their entirety. Briefly, methods are further provided to regulate expression in a plant. The method comprises (a) providing a plant comprising (i) a first polynucleotide construct comprising a polynucleotide encoding a chemically-regulated transcriptional repressor operably linked to a promoter active in said plant, and, (ii) a second polynucleotide construct comprising a polynucleotide of interest operably linked to a first repressible promoter; wherein said first repressible promoter comprises at least one operator, wherein said chemically-regulated transcriptional repressor can bind to said operators in the absence of a chemical ligand and thereby repress transcription from said first repressible promoter in the absence of said chemical ligand, and wherein said plant is tolerant to said chemical ligand; (b) providing the plant with an effective amount of the chemical ligand whereby expression of said polynucleotide of interest are increased.

### XII. Sequence Identity

As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

By “fragment” is intended a portion of the polynucleotide. fragments of a nucleotide sequence may range from at least about 10, about 15, 20 nucleotides, about 50 nucleotides, about 75 nucleotides, about 100 nucleotides, 200 nucleotides, 300 nucleotides, 400 nucleotides, 500 nucleotides, 600 nucleotides, 700 nucleotides and up to the full-length any polynucleotide of the chemical-gene switch system. Methods to assay for the activity of a desired polynucleotide or polypeptide are described elsewhere herein.

“Variants” is intended to mean substantially similar sequences. For polynucleotides or polypeptides, a variant comprises a deletion and/or addition of one or more nucleotides or amino acids at one or more internal sites within the native polynucleotide or polypeptide and/or a substitution of one or more nucleotides or amino acids at one or more sites in the original polynucleotide or original polypeptide. Generally, variants of a particular polynucleotide or polypeptide employed herein having the desired activity will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide or polypeptide as determined by sequence alignment programs and parameters described elsewhere herein.

A nucleic acid or polypeptide is “recombinant” when it is artificial or engineered, or derived from an artificial or engineered protein or nucleic acid. For example, a polynucleotide that is inserted into a vector or any other heterologous location, e.g, in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

An “isolated” or “purified” polynucleotide or polypeptide or biologically active fragment or variant thereof, is substantially free of other cellular material, or culture medium when produced by recombinant techniques, or substantially free of chemical precursors or other chemicals when chemically synthesized. Preferably, an “isolated” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For purposes of the invention, “isolated” when used to refer to nucleic acid molecules excludes isolated chromosomes. For example, in various embodiments, the isolated nucleic acid molecules can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. Non-limiting embodiments include:

1. A recombinant polynucleotide comprising a nucleotide sequence encoding a polypeptide having a sulfonylurea (SU)-dependent stabilization domain.

2. The recombinant polynucleotide of embodiment 1, wherein said SU-dependent stabilization domain comprises
(a) a ligand binding domain of a SU chemically-regulated transcriptional regulator having at least one destabilization mutation;
(b) a DNA binding domain of a SU chemically-regulated transcriptional regulator having at least one destabilization mutation; or
(c) said SU-dependent stabilization domain comprises both (a) and (b).

3. The recombinant polynucleotide of embodiment 1 or 2, wherein the ligand binding domain of the SU chemically-regulated transcriptional regulator comprises a polypeptide having at least 80%, 85%, 90%, or 95% sequence identity to the ligand binding domain of an amino acid sequences sequence set forth in any one of SEQ ID NO:3-419, wherein said polypeptide further comprises at least one destabilization mutation.

4. The recombinant polynucleotide of any one of embodiments 1-3, wherein the encoded polypeptide having the SU-dependent stabilization domain comprises a SU chemically-regulated transcriptional regulator.

5. The recombinant polynucleotide of embodiment 4, wherein the SU chemically-regulated transcriptional regulator comprise a reverse SU chemically-regulated transcriptional repressor (revSuR).

6. The recombinant polynucleotide of embodiment 4, wherein said SuR shares at least 80%, 85%, 90%, or 95% sequence identity to any one of the polypeptides set forth in SEQ ID NO:3-411, wherein said polypeptide further comprises at least one destabilization mutation.

7. The recombinant polynucleotide of embodiment 5, wherein said revSuR shares at least 80%, 85%, 90%, or 95% sequence identity to any one of the polypeptides set forth any one of SEQ ID NO:412-419, wherein said polypeptide further comprises at least one destabilization mutation.

8. The recombinant polynucleotide of embodiment 5 or 7, wherein the revSuR further comprises a transcriptional activator.

9. The recombinant polynucleotide of any one of embodiments 2-7, wherein said destabilization mutation comprises the L17G mutation, the G96R mutation, or any combination thereof.

10. The recombinant polynucleotide of embodiment 8, wherein said destabilization mutation comprises the L17G mutation, the G96R mutation, or any combination thereof.

11. The recombinant polynucleotide of any one of embodiments 1-10, wherein said nucleotide sequence encoding the polypeptide having the SU-dependent stabilization domain is operably linked to a polynucleotide encoding a polypeptide of interest.

12. The recombinant polynucleotide of embodiment 11, further comprises a nucleotide sequence encoding an intein.

13. The recombinant polynucleotide of any one of embodiments 1-12, wherein said SU comprises a pyrimidinylsulfonylurea, a triazinylsulfonylurea, a thiadazolylurea, a chlorosulfuron, an ethametsulfuron, a thifensulfuron, a metsulfuron, a sulfometuron, a tribenuron, a chlorimuron, a nicosulfuron, or a rimsulfuron compound.

14. A DNA construct comprising the polynucleotide of any one of embodiments 1-13, wherein said recombinant polynucleotide is operably linked to a promoter.

15. The DNA construct of embodiment 14, wherein said promoter is a ligand responsive promoter comprising a least one, two or three cognate operators for said encoded SU chemically-regulated transcriptional regulator.

16. The DNA construct of embodiment 15, wherein said cognate operator comprises the tet operator.

17. The DNA construct of embodiment 14, wherein said promoter is a constitutive promoter, tissue-specific promoter, or an inducible promoter.

18. A cell having the recombinant polynucleotide of any one of embodiments 1-14 or the DNA construct of any one of embodiments 15-17.

19. The cell of embodiment 18, wherein said cell is a plant cell.

20. The plant cell of embodiment 19, wherein said plant cell is from a monocot or dicot.

21. The plant cell of embodiment 20, wherein said plant cell is from maize, barley, millet, wheat, rice, sorghum, rye, soybean, canola, alfalfa, sunflower, safflower, sugarcane, tobacco, Arabidopsis, or cotton.

22. A plant comprising the cell of any one of embodiments 19-21.

23. A transgenic seed of the plant of embodiment 22, wherein said seed comprises said recombinant polynucleotide.

24. A recombinant polypeptide encoded by the polynucleotide of any one of embodiments 1-14.

25. A method to modulate the stability of a polypeptide of interest in a cell comprising:

a) providing a cell having a recombinant polynucleotide comprising a nucleotide sequence encoding a polypeptide having a sulfonylurea (SU)-dependent stabilization domain operably linked to a polynucleotide encoding the polypeptide of interest;

b) expressing the recombinant polynucleotide in the cell; and,

c) contacting the cell with an effective amount of a SU ligand, wherein the effective amount of the SU ligand increases the level the polypeptide of interest in the cell.

26. The method of embodiment 25, wherein said recombinant polynucleotide further comprises a nucleotide sequence encoding an intein, wherein the presence of the effective amount of the SU ligand allows for the splicing of the polypeptide of interest from the SU-dependent stabilization domain.

27. The method of embodiment 25 or 26, wherein said SU-dependent stabilization domain comprises
(a) a ligand binding domain of a SU chemically-regulated transcriptional regulator having at least one destabilization mutation;
(b) a DNA binding domain of a SU chemically-regulated transcriptional regulator having at least one destabilization mutation; or
(c) said SU-dependent stabilization domain comprises both (a) and (b).

28. The method of embodiment 27, wherein the SU-dependent stabilization domain comprises a polypeptide having at least 80%, 85%, 90% or 95% sequence identity to the ligand binding domain of an amino acid sequence set forth in any one of SEQ ID NO:3-419, wherein said polypeptide further comprises at least one destabilization mutation.

29. The method of any one of embodiments 25-28, wherein the encoded polypeptide having the SU-dependent stabilization domain comprises a SU chemically-regulated transcriptional regulator.

30. The method of embodiment 29, wherein the SU chemically-regulated transcriptional regulator comprises a reverse SU chemically-regulated transcriptional repressor (revSuR).

31. The method of embodiment 29, wherein said SuR shares at least 80%, 85%, 90%, or 95% sequence identity to any one of the polypeptides set forth any one of SEQ ID NO:3-411, wherein said polypeptide further comprises at least one destabilization mutation.

32. The method of embodiment 30, wherein said revSuR shares at least 80%, 85%, 90%, or 95% sequence identity to any one of the polypeptides set forth any one of SEQ ID NO:412-419, wherein said polypeptide further comprises at least one destabilization mutation.

33. The method of any one of embodiments 30 or 32, wherein the revSuR further comprises a transcriptional activator domain.

34. The method of embodiment 33, wherein said recombinant polynucleotide is operably linked to a promoter comprising at least one, two or three cognate operators for said encoded revSuR.

35. The method of embodiment 34, wherein said cognate operator comprises the tet operator.

36. The method of embodiment 33, wherein said recombinant polynucleotide is operably linked to a constitutive promoter, tissue-specific promoter, or an inducible promoter.

37. The method of any one of embodiments 25-36, wherein said destabilization mutation comprises the L17G mutation, the G96R mutation, or any combination thereof.

38. The method of any of embodiments 25-37, wherein said SU ligand comprises a pyrimidinylsulfonylurea, a triazinylsulfonylurea, a thiadazolylurea, a chlorosulfuron, an ethametsulfuron, a thifensulfuron, a metsulfuron, a sulfometuron, a tribenuron, a chlorimuron, a nicosulfuron, or a rimsulfuron compound.

39. The method of any one of embodiments 25-38, wherein said cell is a plant cell.

40. The method of embodiment 39, wherein said plant cell is in a plant.

41. The method of embodiment 40, wherein said plant cell is a monocot.

42. The method of embodiment 40, wherein said plant cell is a dicot.

43. The method of embodiment 42, wherein said plant cell is from maize, barley, millet, wheat, rice, sorghum, rye, soybean, canola, alfalfa, sunflower, safflower, sugarcane, tobacco, Arabidopsis, or cotton.

44. The method of any one of embodiments 25-43, wherein said chemical ligand is provided by spraying.

45. A cell comprising
a) a first recombinant construct comprising a first promoter operably linked to a SU chemically-regulated transcriptional regulator comprising a reverse SU repressor (revSuR) comprising a transcriptional activator domain, wherein said revSuR comprises a destabilization mutation; and,
b) a second recombinant construct comprising a first ligand responsive promoter comprising at least one, two or three cognate operators for said SU chemically-regulated transcriptional activator operably linked to a polynucleotide of interest.

46. The cell of embodiment 45, wherein said destabilization mutation is found within
(a) a ligand binding domain of the revSuR;
(b) a DNA binding domain of the revSuR; or
(c) both said ligand binding domain and said DNA binding domain.

47. The cell of embodiment 45 or 46, wherein said revSuR shares at least 80%, 85%, 90%, or 95% sequence identity to any one of the polypeptides set forth any one of SEQ ID NO:412-419, wherein said polypeptide further comprises at least one destabilization mutation.

48. The cell of embodiment 45, 46 or 47, wherein said destabilization mutation comprises the L17G mutation, the G96R mutation, or any combination thereof.

49. The cell of any one of embodiments 45-48, wherein said first promoter is a second ligand responsive promoter, a constitutive promoter, tissue-specific promoter, or an inducible promoter.

50. The cell of embodiment 49, wherein said second ligand responsive promoter comprises at least one, two, three, four, five, six, seven or more cognate operators for said revSuR.

51. The cell of any one of embodiments 45-50, wherein said cognate operator comprises the tet operator.

52. The cell of any one of embodiments 45-51, wherein said SU ligand comprises a pyrimidinylsulfonylurea, a triazinylsulfonylurea, a thiadazolylurea, a chlorosulfuron, an ethametsulfuron, a thifensulfuron, a metsulfuron, a sulfometuron, a tribenuron, a chlorimuron, a nicosulfuron, or a rimsulfuron compound.

53. The cell of any one of embodiments 45-52, wherein said cell is a plant cell.

54. The cell of embodiment 53, wherein said plant cell is a monocot or dicot.

55. The cell of embodiment 54, wherein said plant cell is from maize, barley, millet, wheat, rice, sorghum, rye, soybean, canola, alfalfa, sunflower, safflower, sugarcane, tobacco, Arabidopsis, or cotton.

56. The cell of any one of embodiments 53-55, wherein said plant cell is in a plant.

57. A transgenic seed of the plant of embodiment 56, wherein said seed comprises said first and said second recombinant construct.

58. A method to regulate expression in a plant, comprising
(a) providing a cell comprising
   (i) a first recombinant construct comprising a first promoter operably linked to a SU chemically-regulated transcriptional regulator comprising a reverse SU repressor (revSuR) comprising a transcriptional activator domain, wherein said revSuR comprises a destabilization mutation; and,
   (ii) a second recombinant construct comprising a first ligand responsive promoter comprising at least one, two or three cognate operators for said revSuR operably linked to a polynucleotide of interest; and,
(b) providing the cell with an effective amount of the SU ligand whereby the effective amount of the SU ligand increases the level of the revSuR and increases the level of polynucleotide of interest.

59. The method of embodiment 58, wherein said destabilization mutation is found within
(a) a ligand binding domain of the revSuR;
(b) a DNA binding domain of the revSuR; or
(c) both said ligand binding domain and said DNA binding domain.

60. The method of embodiment 58 and 59, wherein said revSuR shares at least 80%, 85%, 90%, or 95% sequence identity to any one of the polypeptides set forth any one of SEQ ID NO:412-419, wherein said polypeptide further comprises at least one destabilization mutation.

61. The method of embodiment 58, 59, or 60, wherein said destabilization mutation comprises the L17G mutation, the G96R mutation, or any combination thereof.

62. The method of any one of embodiments 58-61, wherein said first promoter is a second ligand responsive promoter.

63. The method of embodiment 62, wherein said second ligand responsive promoter comprises at least one, two or three cognate operators for said revSuR.

64. The method of any one of embodiments 58-63, wherein said cognate operator comprises the tet operator.

65. The method of any one of embodiments 58-64, wherein said SU ligand comprises a pyrimidinylsulfonylurea, a triazinylsulfonylurea, a thiadazolylurea, a chlorosulfuron, an ethametsulfuron, a thifensulfuron, a metsulfuron, a sulfometuron, a tribenuron, a chlorimuron, a nicosulfuron, or a rimsulfuron compound.

66. The method of any one of embodiments 58-65, wherein said cell is a plant cell.

67. The method of embodiment 66, wherein said plant cell is a monocot or dicot.

68. The method of embodiment 67, wherein said plant cell is from maize, barley, millet, wheat, rice, sorghum, rye, soybean, canola, alfalfa, sunflower, safflower, sugarcane, tobacco, Arabidopsis, or cotton.

69. The method of any one of embodiments 66-68, wherein said plant cell is in a plant.

TABLE 1A
Summary of SEQ ID NOS.

| SEQ ID NO | Brief Description |
|---|---|
| 1 | Amino acid sequence of TetR(B) |
| 2 | Amino acid sequence of a variant of SEQ ID NO: 1 |
| 3-13 | Amino acid sequence for some SuR polypeptides |
| 14-204 | Amino acid sequence for SuR polypeptides that can employ |
| | ethametsulfuron as a SU ligand |
| 205-419 | Amino acid sequence for SuR polypeptides that can employ |
| | chlorsulfuron a SU ligand. |
| 412-419 | Amino acid sequence of SuR polypeptides with reverse |
| | repressor activity |
| 420-430 | Nucleic acid sequence encoding SEQ ID NO: 3-13 |
| 431-621 | Nucleic acid sequence encoding SEQ ID NO: 431-621 |
| 622-836 | Nucleic acid sequence encoding SEQ ID NO: 405-419 |
| 837-840 | oligonucleotides |
| 841-847 | Various constructs |
| 848 | Tet operator sequence |
| 849 | Plant actin promoter |
| 850 | banana streak virus promoter (BSV) |
| 851 | a mirabilis mosaic virus promoter |
| 852 | enhanced MMV promoter (dMMV) |
| 853 | plant P450 promoter (MP1) |
| 854 | elongation factor la (EF1A) promoter |
| 855 | Plant actin promoter with tet op (actin/Op) |
| 856 | Banana steak virus promoter with tet op (BSV/Op) |
| 857 | mirabilis mosaic virus promoter with tet op (MMV/Op) |
| 858 | enhanced MMV promoter with tet op (dMMV/Op) |
| 859 | plant P450 promoter with tet op (MP1/0p) |
| 860 | elongation factor la promoter with tet op (EF1A/Op) |
| 861 | 35S CaMV promoter with ADH1 intron |
| 862 | 35S CaMV promoter engineered with tet operators |
| 863 | Amino acid sequence for L13-23, an EsR |
| 864 | Amino acid sequence for L15-20, an EsR |
| 865 | Amino acid sequence for L15-20-M4, an EsR |
| 866 | Amino acid sequence for L15-20-M9, an EsR |
| 867 | Amino acid sequence for L15-20-M34, an EsR |
| 868 | Amino acid sequence for CsL4.2-20, an CsR having the |
| | L17G mutation |
| 869 | Amino acid sequence for CsL4.2-15, an CsR |
| 870 | Amino acid sequence for CsL4.2-20, an CsR |
| 871-883 | Various oligonucleotides |
| 884 | Amino acid sequence for L13-23, an EsR, having the L17G |
| | mutation |
| 885 | Amino acid sequence for L15-20, an EsR having the L17G |
| | mutation |
| 886 | Amino acid sequence for L15-20-M4, an EsR having the |
| | L17G mutation |
| 887 | Amino acid sequence for L15-20-M9, an EsR having the |
| | L17G mutation |
| 888 | Amino acid sequence for L15-20-M34, an EsR having the |
| | L17G mutation |
| 889 | Amino acid sequence for CsL4.2-15, an CsR having the |
| | L17G mutation |

TABLE 1B
Additional information on SEQ ID NOS

| SEQ ID | | Description/clone |
|---|---|---|
| NO | type | name |
| 3 | AA | L1-02 |
| 4 | AA | L1-07 |
| 5 | AA | L1-09 |
| 6 | AA | L1-20 |
| 7 | AA | L1-22 |
| 8 | AA | L1-24 |
| 9 | AA | L1-28 |
| 10 | AA | L1-29 |
| 11 | AA | L1-31 |
| 12 | AA | L1-38 |
| 13 | AA | L1-44 |
| 14 | AA | L6-1B03 |
| 15 | AA | L6-1C03 |
| 16 | AA | L6-1C06 |
| 17 | AA | L6-1G06 |
| 18 | AA | L6-1G07 |
| 19 | AA | L6-1G09 |
| 20 | AA | L6-1G10 |
| 21 | AA | L6-1G11 |
| 22 | AA | L6-1H12 |
| 23 | AA | L6-2A01 |
| 24 | AA | L6-2A02 |
| 25 | AA | L6-2A04 |
| 26 | AA | L6-2A06 |
| 27 | AA | L6-2A12 |
| 28 | AA | L6-2B04 |
| 29 | AA | L6-2B06 |
| 30 | AA | L6-2B08 |
| 31 | AA | L6-2B09 |
| 32 | AA | L6-2B10 |
| 33 | AA | L6-2B11 |
| 34 | AA | L6-2C02 |
| 35 | AA | L6-2C05 |
| 36 | AA | L6-2C09 |
| 37 | AA | L6-2C10 |
| 38 | AA | L6-2C11 |
| 39 | AA | L6-2D01 |
| 40 | AA | L6-2D02 |
| 41 | AA | L6-2D03 |
| 42 | AA | L6-2D04 |
| 43 | AA | L6-2D07 |
| 44 | AA | L6-2D11 |
| 45 | AA | L6-2D12 |
| 46 | AA | L6-2E02 |
| 47 | AA | L6-2E03 |
| 48 | AA | L6-2E04 |
| 49 | AA | L6-2E05 |
| 50 | AA | L6-2E07 |
| 51 | AA | L6-2E08 |
| 52 | AA | L6-2E09 |
| 53 | AA | L6-2E11 |
| 54 | AA | L6-2F08 |
| 55 | AA | L6-2F10 |
| 56 | AA | L6-2F11 |
| 57 | AA | L6-2F12 |
| 58 | AA | L6-2G01 |
| 59 | AA | L6-2G02 |
| 60 | AA | L6-2G03 |
| 61 | AA | L6-2G05 |
| 62 | AA | L6-2G10 |
| 63 | AA | L6-2H01 |
| 64 | AA | L6-2H02 |
| 65 | AA | L6-2H03 |
| 66 | AA | L6-2H04 |
| 67 | AA | L6-2H06 |
| 68 | AA | L6-2H07 |
| 69 | AA | L6-2H10 |
| 70 | AA | L6-2H11 |
| 71 | AA | L6-3A01 |
| 72 | AA | L6-3A02 |
| 73 | AA | L6-3A03 |
| 74 | AA | L6-3A06 |
| 75 | AA | L6-3A11 |
| 76 | AA | L6-3B08 |
| 77 | AA | L6-3B09 |
| 78 | AA | L6-3C02 |
| 79 | AA | L6-3C04 |
| 80 | AA | L6-3C05 |
| 81 | AA | L6-3C06 |
| 82 | AA | L6-3D03 |
| 83 | AA | L6-3D05 |
| 84 | AA | L6-3D09 |
| 85 | AA | L6-3E08 |
| 86 | AA | L6-3E09 |
| 87 | AA | L6-3E10 |
| 88 | AA | L6-3F02 |
| 89 | AA | L6-3F09 |
| 90 | AA | L6-3F12 |
| 91 | AA | L6-3G03 |
| 92 | AA | L6-3G05 |
| 93 | AA | L6-3G09 |
| 94 | AA | L6-3H02 |
| 95 | AA | L6-3H05 |
| 96 | AA | L6-3H08 |
| 97 | AA | L6-4A01 |
| 98 | AA | L6-4A03 |
| 99 | AA | L6-4A04 |
| 100 | AA | L6-4A09 |
| 101 | AA | L6-4A10 |
| 102 | AA | L6-4A11 |
| 103 | AA | L6-4B05 |
| 104 | AA | L6-4B06 |
| 105 | AA | L6-4B07 |
| 106 | AA | L6-4B08 |
| 107 | AA | L6-4B12 |
| 108 | AA | L6-4C01 |
| 109 | AA | L6-4C03 |
| 110 | AA | L6-4C04 |
| 111 | AA | L6-4C07 |
| 112 | AA | L6-4C08 |
| 113 | AA | L6-4C09 |
| 114 | AA | L6-4C10 |
| 115 | AA | L6-4C11 |
| 116 | AA | L6-4D09 |
| 117 | AA | L6-4D10 |
| 118 | AA | L6-4E01 |
| 119 | AA | L6-4E02 |
| 120 | AA | L6-4E03 |
| 121 | AA | L6-4E05 |
| 122 | AA | L6-4E08 |
| 123 | AA | L6-4E09 |
| 124 | AA | L6-4E11 |
| 125 | AA | L6-4E12 |
| 126 | AA | L6-4F01 |
| 127 | AA | L6-4F10 |
| 128 | AA | L6-4F12 |
| 129 | AA | L6-4G02 |
| 130 | AA | L6-4G03 |
| 131 | AA | L6-4G06 |
| 132 | AA | L6-4G07 |
| 133 | AA | L6-4G08 |
| 134 | AA | L6-4G10 |
| 135 | AA | L6-4H07 |
| 136 | AA | L6-5A02 |
| 137 | AA | L6-5A03 |
| 138 | AA | L6-5A04 |
| 139 | AA | L6-5A05 |
| 140 | AA | L6-5A06 |
| 141 | AA | L6-5A07 |
| 142 | AA | L6-5A09 |
| 143 | AA | L6-5A10 |
| 144 | AA | L6-5B02 |
| 145 | AA | L6-5B07 |
| 146 | AA | L6-5B08 |
| 147 | AA | L6-5B11 |
| 148 | AA | L6-5C01 |
| 149 | AA | L6-5C02 |
| 150 | AA | L6-5C04 |
| 151 | AA | L6-5C08 |
| 152 | AA | L6-5C10 |
| 153 | AA | L6-5C11 |
| 154 | AA | L6-5D04 |
| 155 | AA | L6-5D09 |
| 156 | AA | L6-5D11 |
| 157 | AA | L6-5D12 |
| 158 | AA | L6-5E05 |
| 159 | AA | L6-5E09 |
| 160 | AA | L6-5F02 |
| 161 | AA | L6-5F04 |
| 162 | AA | L6-5F05 |
| 163 | AA | L6-5F07 |
| 164 | AA | L6-5F08 |
| 165 | AA | L6-5F10 |
| 166 | AA | L6-5F12 |
| 167 | AA | L6-5G03 |
| 168 | AA | L6-5G05 |
| 169 | AA | L6-5G06 |
| 170 | AA | L6-5G08 |
| 171 | AA | L6-5G11 |
| 172 | AA | L6-5G12 |
| 173 | AA | L6-5H03 |
| 174 | AA | L6-5H06 |
| 175 | AA | L6-5H07 |
| 176 | AA | L6-5H12 |
| 177 | AA | L6-6A09 |
| 178 | AA | L6-6B01 |
| 179 | AA | L6-6B03 |
| 180 | AA | L6-6B04 |
| 181 | AA | L6-6B05 |
| 182 | AA | L6-6B10 |
| 183 | AA | L6-6C01 |
| 184 | AA | L6-6C02 |
| 185 | AA | L6-6C04 |
| 186 | AA | L6-6C05 |
| 187 | AA | L6-6C06 |
| 188 | AA | L6-6C07 |
| 189 | AA | L6-6C10 |
| 190 | AA | L6-6C11 |
| 191 | AA | L6-6D02 |
| 192 | AA | L6-6D06 |
| 193 | AA | L6-6D07 |
| 194 | AA | L6-6D09 |
| 195 | AA | L6-6D10 |
| 196 | AA | L6-6D12 |
| 197 | AA | L6-6E01 |
| 198 | AA | L6-6E02 |
| 199 | AA | L6-6E03 |
| 200 | AA | L6-6E11 |
| 201 | AA | L6-6F03 |
| 202 | AA | L6-6F07 |
| 203 | AA | L6-6F08 |
| 204 | AA | L6-6G01 |
| 205 | AA | L7-1A01 |
| 206 | AA | L7-1B01 |
| 207 | AA | L7-1C01 |
| 208 | AA | L7-1D01 |
| 209 | AA | L7-1E01 |
| 210 | AA | L7-1F01 |
| 211 | AA | L7-1G01 |
| 212 | AA | L7-1C02 |
| 213 | AA | L7-1D02 |
| 214 | AA | L7-1E02 |
| 215 | AA | L7-1F02 |
| 216 | AA | L7-1G02 |
| 217 | AA | L7-1H02 |
| 218 | AA | L7-1C03 |
| 219 | AA | L7-1E03 |
| 220 | AA | L7-1A04 |
| 221 | AA | L7-1C04 |
| 222 | AA | L7-1D04 |
| 223 | AA | L7-1E04 |
| 224 | AA | L7-1F04 |
| 225 | AA | L7-1G04 |
| 226 | AA | L7-1H04 |
| 227 | AA | L7-1A05 |
| 228 | AA | L7-1C05 |
| 229 | AA | L7-1E05 |
| 230 | AA | L7-1F05 |
| 231 | AA | L7-1A06 |
| 232 | AA | L7-1B06 |
| 233 | AA | L7-1D06 |
| 234 | AA | L7-1E06 |
| 235 | AA | L7-1F06 |
| 236 | AA | L7-1G06 |
| 237 | AA | L7-1H06 |
| 238 | AA | L7-1A07 |
| 239 | AA | L7-1B07 |
| 240 | AA | L7-1C07 |
| 241 | AA | L7-1D07 |
| 242 | AA | L7-1E07 |
| 243 | AA | L7-1F07 |
| 244 | AA | L7-1G07 |
| 245 | AA | L7-1A08 |
| 246 | AA | L7-1C08 |
| 247 | AA | L7-1D08 |
| 248 | AA | L7-1E08 |
| 249 | AA | L7-1F08 |
| 250 | AA | L7-1G08 |
| 251 | AA | L7-1A09 |
| 252 | AA | L7-1B09 |
| 253 | AA | L7-1C09 |
| 254 | AA | L7-1D09 |
| 255 | AA | L7-1E09 |
| 256 | AA | L7-1G09 |
| 257 | AA | L7-1A10 |
| 258 | AA | L7-1B10 |
| 259 | AA | L7-1C10 |
| 260 | AA | L7-1D10 |
| 261 | AA | L7-1F10 |
| 262 | AA | L7-1A11 |
| 263 | AA | L7-1B11 |
| 264 | AA | L7-1C11 |
| 265 | AA | L7-1E11 |
| 266 | AA | L7-1A12 |
| 267 | AA | L7-1C12 |
| 268 | AA | L7-1F12 |
| 269 | AA | L7-1G12 |
| 270 | AA | L7-2A01 |
| 271 | AA | L7-2B01 |
| 272 | AA | L7-2D01 |
| 273 | AA | L7-2E01 |
| 274 | AA | L7-2F01 |
| 275 | AA | L7-2G01 |
| 276 | AA | L7-2H01 |
| 277 | AA | L7-2B02 |
| 278 | AA | L7-2D02 |
| 279 | AA | L7-2E02 |
| 280 | AA | L7-2F02 |
| 281 | AA | L7-2G02 |
| 282 | AA | L7-2H02 |
| 283 | AA | L7-2D03 |
| 284 | AA | L7-2E03 |
| 285 | AA | L7-2F03 |
| 286 | AA | L7-2G03 |
| 287 | AA | L7-2H03 |
| 288 | AA | L7-2D04 |
| 289 | AA | L7-2E04 |
| 290 | AA | L7-2F04 |
| 291 | AA | L7-2H04 |
| 292 | AA | L7-2B05 |
| 293 | AA | L7-2D05 |
| 294 | AA | L7-2E05 |
| 295 | AA | L7-2F05 |
| 296 | AA | L7-2H05 |
| 297 | AA | L7-2A06 |
| 298 | AA | L7-2C06 |
| 299 | AA | L7-2D06 |
| 300 | AA | L7-2F06 |
| 301 | AA | L7-2G06 |
| 302 | AA | L7-2A07 |
| 303 | AA | L7-2B07 |
| 304 | AA | L7-2C07 |
| 305 | AA | L7-2D07 |
| 306 | AA | L7-2E07 |
| 307 | AA | L7-2G07 |
| 308 | AA | L7-2B08 |
| 309 | AA | L7-2D08 |
| 310 | AA | L7-2F08 |
| 311 | AA | L7-2G08 |
| 312 | AA | L7-2B09 |
| 313 | AA | L7-2C09 |
| 314 | AA | L7-2E09 |
| 315 | AA | L7-2B10 |
| 316 | AA | L7-2E10 |
| 317 | AA | L7-2G10 |
| 318 | AA | L7-2C11 |
| 319 | AA | L7-2D11 |
| 320 | AA | L7-2F11 |
| 321 | AA | L7-2G11 |
| 322 | AA | L7-2B12 |
| 323 | AA | L7-2C12 |
| 324 | AA | L7-2D12 |
| 325 | AA | L7-2F12 |
| 326 | AA | L7-2G12 |
| 327 | AA | L7-3A01 |
| 328 | AA | L7-3C01 |
| 329 | AA | L7-3G01 |
| 330 | AA | L7-3H01 |
| 331 | AA | L7-3A02 |
| 332 | AA | L7-3B02 |
| 333 | AA | L7-3D02 |
| 334 | AA | L7-3G02 |
| 335 | AA | L7-3H02 |
| 336 | AA | L7-3B03 |
| 337 | AA | L7-3C03 |
| 338 | AA | L7-3E03 |
| 339 | AA | L7-3G03 |
| 340 | AA | L7-3H03 |
| 341 | AA | L7-3B04 |
| 342 | AA | L7-3E04 |
| 343 | AA | L7-3G04 |
| 344 | AA | L7-3A05 |
| 345 | AA | L7-3B05 |
| 346 | AA | L7-3H05 |
| 347 | AA | L7-3B06 |
| 348 | AA | L7-3D06 |
| 349 | AA | L7-3E06 |
| 350 | AA | L7-3A07 |
| 351 | AA | L7-3C07 |
| 352 | AA | L7-3F07 |
| 353 | AA | L7-3A08 |
| 354 | AA | L7-3B08 |
| 355 | AA | L7-3C08 |
| 356 | AA | L7-3F08 |
| 357 | AA | L7-3G08 |
| 358 | AA | L7-3B09 |
| 359 | AA | L7-3F09 |
| 360 | AA | L7-3A10 |
| 361 | AA | L7-3B10 |
| 362 | AA | L7-3C10 |
| 363 | AA | L7-3G10 |
| 364 | AA | L7-3A11 |
| 365 | AA | L7-3C11 |
| 366 | AA | L7-3E11 |
| 367 | AA | L7-3G11 |
| 368 | AA | L7-3A12 |
| 369 | AA | L7-3B12 |
| 370 | AA | L7-3C12 |
| 371 | AA | L7-3E12 |
| 372 | AA | L7-3F12 |
| 373 | AA | L7-3G12 |
| 374 | AA | L7-4A01 |
| 375 | AA | L7-4A03 |
| 376 | AA | L7-4A04 |
| 377 | AA | L7-4A06 |
| 378 | AA | L7-4A08 |
| 379 | AA | L7-4A09 |
| 380 | AA | L7-4A12 |
| 381 | AA | L7-4B03 |
| 382 | AA | L7-4B04 |
| 383 | AA | L7-4B06 |
| 384 | AA | L7-4B07 |
| 385 | AA | L7-4C01 |
| 386 | AA | L7-4C03 |
| 387 | AA | L7-4C04 |
| 388 | AA | L7-4C06 |
| 389 | AA | L7-4C09 |
| 390 | AA | L7-4C12 |
| 391 | AA | L7-4D04 |
| 392 | AA | L7-4D07 |
| 393 | AA | L7-4D08 |
| 394 | AA | L7-4D10 |
| 395 | AA | L7-4D11 |
| 396 | AA | L7-4E01 |
| 397 | AA | L7-4E02 |
| 398 | AA | L7-4E04 |
| 399 | AA | L7-4E05 |
| 400 | AA | L7-4E07 |
| 401 | AA | L7-4E08 |
| 402 | AA | L6-3A09 |
| 403 | AA | L7-4C06 |
| 404 | AA | L10-84 |
| 405 | AA | L13-2-46 |
| 406 | AA | L12-1-10 |
| 407 | AA | L13-2-23 |
| 408 | AA | L7-1C3-A5 |
| 409 | AA | L7-1F8-A11 |
| 410 | AA | L7-1G6-B2 |
| 411 | AA | L7-3E3-D1 |
| 412 | AA | L1-18 |
| 413 | AA | L1-21 |
| 414 | AA | L1-25 |
| 415 | AA | L1-33 |
| 416 | AA | L1-34 |
| 417 | AA | L1-36 |
| 418 | AA | L1-39 |
| 419 | AA | L1-41 |
| 420 | DNA | L1-02 CDS |
| 421 | DNA | L1-07 CDS |
| 422 | DNA | L1-09 CDS |
| 423 | DNA | L1-20 CDS |
| 424 | DNA | L1-22 CDS |
| 425 | DNA | L1-24 CDS |
| 426 | DNA | L1-28 CDS |
| 427 | DNA | L1-29 CDS |
| 428 | DNA | L1-31 CDS |
| 429 | DNA | L1-38 CDS |
| 430 | DNA | L1-44 CDS |
| 431 | DNA | L6-1B03 CDS |
| 432 | DNA | L6-1C03 CDS |
| 433 | DNA | L6-1C06 CDS |
| 434 | DNA | L6-1G06 CDS |
| 435 | DNA | L6-1G07 CDS |
| 436 | DNA | L6-1G09 CDS |
| 437 | DNA | L6-1G10 CDS |
| 438 | DNA | L6-1G11 CDS |
| 439 | DNA | L6-1H12 CDS |
| 440 | DNA | L6-2A01 CDS |
| 441 | DNA | L6-2A02 CDS |
| 442 | DNA | L6-2A04 CDS |
| 443 | DNA | L6-2A06 CDS |
| 444 | DNA | L6-2A12 CDS |
| 445 | DNA | L6-2B04 CDS |
| 446 | DNA | L6-2B06 CDS |
| 447 | DNA | L6-2B08 CDS |
| 448 | DNA | L6-2B09 CDS |
| 449 | DNA | L6-2B10 CDS |
| 450 | DNA | L6-2B11 CDS |
| 451 | DNA | L6-2C02 CDS |
| 452 | DNA | L6-2C05 CDS |
| 453 | DNA | L6-2C09 CDS |
| 454 | DNA | L6-2C10 CDS |
| 455 | DNA | L6-2C11 CDS |
| 456 | DNA | L6-2D01 CDS |
| 457 | DNA | L6-2D02 CDS |
| 458 | DNA | L6-2D03 CDS |
| 459 | DNA | L6-2D04 CDS |
| 460 | DNA | L6-2D07 CDS |
| 461 | DNA | L6-2D11 CDS |
| 462 | DNA | L6-2D12 CDS |
| 463 | DNA | L6-2E02 CDS |
| 464 | DNA | L62E03 CDS |
| 465 | DNA | L6-2E04 CDs |
| 466 | DNA | L6-2E05 CDS |
| 467 | DNA | L6-2E07 CDS |
| 468 | DNA | L6-2E08 CDS |
| 469 | DNA | L6-2E09 CDS |
| 470 | DNA | L6-2E11 CDS |
| 471 | DNA | L6-2F08 CDS |
| 472 | DNA | L6-2F10 CDS |
| 473 | DNA | L6-2F11 CDS |
| 474 | DNA | L6-2F12 CDS |
| 475 | DNA | L6-2G01 CDS |
| 476 | DNA | L6-2G02 CDS |
| 477 | DNA | L6-2G03 CDS |
| 478 | DNA | L6-2G05 CDS |
| 479 | DNA | L6-2G10 CDS |
| 480 | DNA | L6-2H01 CDS |
| 481 | DNA | L6-2H02 CDS |
| 482 | DNA | L6-2H03 CDS |
| 483 | DNA | L6-2H04 CDS |
| 484 | DNA | L6-2H06 CDS |
| 485 | DNA | L6-2H07 CDS |
| 486 | DNA | L6-2H10 CDS |
| 487 | DNA | L6-2H11 CDS |
| 488 | DNA | L6-3A01 CDS |
| 489 | DNA | L6-3A02 CDS |
| 490 | DNA | L6-3A03 CDS |
| 491 | DNA | L6-3A06 CDS |
| 492 | DNA | L6-3A11 CDS |
| 493 | DNA | L6-3B08 CDS |
| 494 | DNA | L6-3B09 CDS |
| 495 | DNA | L6-3C02 CDS |
| 496 | DNA | L6-3C04 CDS |
| 497 | DNA | L6-3C05 CDS |
| 498 | DNA | L6-3C06 CDS |
| 499 | DNA | L6-3D03 CDS |
| 500 | DNA | L6-3D05 CDS |
| 501 | DNA | L6-3D09 CDS |
| 502 | DNA | L6-3E08 CDS |
| 503 | DNA | L6-3E09 CDS |
| 504 | DNA | L6-3E10 CDS |
| 505 | DNA | L6-3F02 CDS |
| 506 | DNA | L6-3F09 CDS |
| 507 | DNA | L6-3F12 CDS |
| 508 | DNA | L6-3G03 CDS |
| 509 | DNA | L6-3G05 CDS |
| 510 | DNA | L6-3G09 CDS |
| 511 | DNA | L6-3H02 CDS |
| 512 | DNA | L6-3H05 CDS |
| 513 | DNA | L6-3H08 CDS |
| 514 | DNA | L6-4A01 CDS |
| 515 | DNA | L6-4A03 CDS |
| 516 | DNA | L6-4A04 CDS |
| 517 | DNA | L6-4A09 CDS |
| 518 | DNA | L6-4A10 CDS |
| 519 | DNA | L6-4A11 CDS |
| 520 | DNA | L6-4B05 CDS |
| 521 | DNA | L6-4B06 CDS |
| 522 | DNA | L6-4B07 CDS |
| 523 | DNA | L6-4B08 CDS |
| 524 | DNA | L6-4B12 CDS |
| 525 | DNA | L6-4C01 CDS |
| 526 | DNA | L6-4C03 CDS |
| 527 | DNA | L6-4C04 CDS |
| 528 | DNA | L6-4C07 CDS |
| 529 | DNA | L6-4C08 CDS |
| 530 | DNA | L6-4C09 CDS |
| 531 | DNA | L6-4C10 CDS |
| 532 | DNA | L6-4C11 CDS |
| 533 | DNA | L6-4D09 CDS |
| 534 | DNA | L6-4D10 CDS |
| 535 | DNA | L6-4E01 CDS |
| 536 | DNA | L6-4E02 CDS |
| 537 | DNA | L6-4E03 CDS |
| 538 | DNA | L6-4E05 CDS |
| 539 | DNA | L6-4E08 CDS |
| 540 | DNA | L6-4E09 CDS |
| 541 | DNA | L6-4E11 CDS |
| 542 | DNA | L6-4E12 CDS |
| 543 | DNA | L6-4F01 CDS |
| 544 | DNA | L6-4F10 CDS |
| 545 | DNA | L6-4F12 CDS |
| 546 | DNA | L6-4G02 CDS |
| 547 | DNA | L6-4G03 CDS |
| 548 | DNA | L6-4G06 CDS |
| 549 | DNA | L6-4G07 CDS |
| 550 | DNA | L6-4G08 CDS |
| 551 | DNA | L6-4G10 CDS |
| 552 | DNA | L6-4H07 CDS |
| 553 | DNA | L6-5A02 CDS |
| 554 | DNA | L6-5A03 CDS |
| 555 | DNA | L6-5A04 CDS |
| 556 | DNA | L6-5A05 CDS |
| 557 | DNA | L6-5A06 CDS |
| 558 | DNA | L6-5A07 CDS |
| 559 | DNA | L6-5A09 CDS |
| 560 | DNA | L6-5A10 CDS |
| 561 | DNA | L6-5B02 CDS |
| 562 | DNA | L6-5B07 CDS |
| 563 | DNA | L6-5B08 CDS |
| 564 | DNA | L6-5B11 CDS |
| 565 | DNA | L6-5C01 CDS |
| 566 | DNA | L6-5C02 CDS |
| 567 | DNA | L6-5C04 CDS |
| 568 | DNA | L6-5C08 CDS |
| 569 | DNA | L6-5C10 CDS |
| 570 | DNA | L6-5C11 CDS |
| 571 | DNA | L6-5D04 CDS |
| 572 | DNA | L6-5D09 CDS |
| 573 | DNA | L6-5D11 CDS |
| 574 | DNA | L6-5D12 CDS |
| 575 | DNA | L6-5E05 CDS |
| 576 | DNA | L6-5E09 CDS |
| 577 | DNA | L6-5F02 CDS |
| 578 | DNA | L6-5F04 CDS |
| 579 | DNA | L6-5F05 CDS |
| 580 | DNA | L6-5F07 CDS |
| 581 | DNA | L6-5F08 CDS |
| 582 | DNA | L6-5F10 CDS |
| 583 | DNA | L6-5F12 CDS |
| 584 | DNA | L6-5G03 CDS |
| 585 | DNA | L6-5G05 CDS |
| 586 | DNA | L6-5G06 CDS |
| 587 | DNA | L0-5G08 CDS |
| 588 | DNA | L6-5G11 CDS |
| 589 | DNA | L6-5G12 CDS |
| 590 | DNA | L6-5H03 CDS |
| 591 | DNA | L6-5H06 CDS |
| 592 | DNA | L6-5H07 CDS |
| 593 | DNA | L6-5H12 CDS |
| 594 | DNA | L6-6A09 CDS |
| 595 | DNA | L6-6B01 CDS |
| 596 | DNA | L6-6B03 CDS |
| 597 | DNA | L6-6B04 CDS |
| 598 | DNA | L6-6B05 CDS |
| 599 | DNA | L6-6B10 CDS |
| 600 | DNA | L6-6C01 CDS |
| 601 | DNA | L6-6C02 CDS |
| 602 | DNA | L6-6C04 CDS |
| 603 | DNA | L6-6C05 CDS |
| 604 | DNA | L6-6C06 CDS |
| 605 | DNA | L6-6C07 CDS |
| 606 | DNA | L6-6C10 CDS |
| 607 | DNA | L6-6C11 CDS |
| 608 | DNA | L6-6D02 CDS |
| 609 | DNA | L6-6D06 CDS |
| 610 | DNA | L6-6D07 CDS |
| 611 | DNA | L6-6D09 CDS |
| 612 | DNA | L6-6D10 CDS |
| 613 | DNA | L6-6D12 CDS |
| 614 | DNA | L6-6E01 CDS |
| 615 | DNA | L6-6E02 CDS |
| 616 | DNA | L6-6E03 CDS |
| 617 | DNA | L6-6E11CDS |
| 618 | DNA | L6-6F03 CDS |
| 619 | DNA | L6-6F07 CDS |
| 620 | DNA | L6-6F08 CDS |
| 621 | DNA | L6-6G01 CDS |
| 622 | DNA | L7-1A01 CDS |
| 623 | DNA | L7-1B01 Cds |
| 624 | DNA | L7-1C01 CDS |
| 625 | DNA | L7-1D01 CDS |
| 626 | DNA | L7-1E01 CDS |
| 627 | DNA | L7-1F01 CDS |
| 628 | DNA | L7-1G01 CDS |
| 629 | DNA | L7-1C02 CDS |
| 630 | DNA | L7-1D02 CDS |
| 631 | DNA | L7-1E02 CDS |
| 632 | DNA | L7-1F02 CDS |
| 633 | DNA | L7-1G02 CDS |
| 634 | DNA | L7-1H02 CDS |
| 635 | DNA | L7-1C03 CDS |
| 636 | DNA | L7-1E03 CDS |
| 637 | DNA | L7-1A04 CDS |
| 638 | DNA | L7-1C04 CDS |
| 639 | DNA | L7-1D04 CDS |
| 640 | DNA | L7-1E04 CDS |
| 641 | DNA | L7-1F04 CDS |
| 642 | DNA | L7-1G04 CDS |
| 643 | DNA | L7-1H04 CDS |
| 644 | DNA | L7-1A05 CDS |
| 645 | DNA | L7-1C05 CDS |
| 646 | DNA | L7-1E05 CDS |
| 647 | DNA | L7-1F05 CDS |
| 648 | DNA | L7-1A06 CDS |
| 649 | DNA | L7-1B06 CDS |
| 650 | DNA | L7-1D06 CDS |
| 651 | DNA | L7-1E06 CDS |
| 652 | DNA | L7-1F06 CDS |
| 653 | DNA | L7-1G06 CDS |
| 654 | DNA | L7-1H06 CDS |
| 655 | DNA | L7-1A07 CDS |
| 656 | DNA | L7-1B07 CDS |
| 657 | DNA | L7-1C07 CDS |
| 658 | DNA | L7-1D07 CDS |
| 659 | DNA | L7-1E07 CDS |
| 660 | DNA | L7-1F07 CDS |
| 661 | DNA | L7-1G07 CDS |
| 662 | DNA | L7-1A08 CDS |
| 663 | DNA | L7-1C08 CDS |
| 664 | DNA | L7-1D08 CDS |
| 665 | DNA | L7-1E08 CDS |
| 666 | DNA | L7-1F08 CDS |
| 667 | DNA | L7-1G08 CDS |
| 668 | DNA | L7-1A09 CDS |
| 669 | DNA | L7-1B09 CDS |
| 670 | DNA | L7-1C09 CDS |
| 671 | DNA | L7-1D09 CDS |
| 672 | DNA | L7-1E09 CDS |
| 673 | DNA | L7-1G09 CDS |
| 674 | DNA | L7-1A10 CDS |
| 675 | DNA | L7-1B10 CDS |
| 676 | DNA | L7-1C10 CDS |
| 677 | DNA | L7-1D10 CDS |
| 678 | DNA | L7-1F10 CDS |
| 679 | DNA | L7-1A11 CDS |
| 680 | DNA | L7-1B11 CDS |
| 681 | DNA | L7-1C11 CDS |
| 682 | DNA | L7-1E11 CDS |
| 683 | DNA | L7-1A12 CDS |
| 684 | DNA | L7-1C12 CDS |
| 685 | DNA | L7-1F12 CDS |
| 686 | DNA | L7-1G12 CDS |
| 687 | DNA | L7-2A01 CDS |
| 688 | DNA | L7-2B01 CDS |
| 689 | DNA | L7-2D01 CDS |
| 690 | DNA | L7-2E01 CDS |
| 691 | DNA | L7-2F01 CDS |
| 692 | DNA | L7-2G01 CDS |
| 693 | DNA | L7-2H01 CDS |
| 694 | DNA | L7-2B02 CDS |
| 695 | DNA | L7-2D02 CDS |
| 696 | DNA | L7-2E02 CDS |
| 697 | DNA | L7-2F02 CDS |
| 698 | DNA | L7-2G02 CDS |
| 699 | DNA | L7-2H02 CDS |
| 700 | DNA | L7-2D03 CDS |
| 701 | DNA | L7-2E03 CDS |
| 702 | DNA | L7-2F03 CDS |
| 703 | DNA | L7-2G03 CDS |
| 704 | DNA | L7-2H03 CDS |
| 705 | DNA | L7-2D04 CDS |
| 706 | DNA | L7-2E04 CDS |
| 707 | DNA | L7-2F04 CDS |
| 708 | DNA | L7-2H04 CDS |
| 709 | DNA | L7-2B05 CDS |
| 710 | DNA | L7-2D05 CDS |
| 711 | DNA | L7-2E05 CDS |
| 712 | DNA | L7-2F05 CDS |
| 713 | DNA | L7-2H05 CDS |
| 714 | DNA | L7-2A06 CDS |
| 715 | DNA | L7-2C06 CDS |
| 716 | DNA | L7-2D06 CDS |
| 717 | DNA | L7-2F06 CDS |
| 718 | DNA | L7-2G06 CDS |
| 719 | DNA | L7-2A07 CDS |
| 720 | DNA | L7-2B07 CDS |
| 721 | DNA | L7-2C07 CDS |
| 722 | DNA | L7-2D07 CDS |
| 723 | DNA | L7-2E07 CDS |
| 724 | DNA | L7-2G07 CDS |
| 725 | DNA | L7-2B08 CDS |
| 726 | DNA | L7-2D08 CDS |
| 727 | DNA | L7-2F08 CDS |
| 728 | DNA | L7-2G08 CDS |
| 729 | DNA | L7-2B09 CDS |
| 730 | DNA | L7-2C09 CDS |
| 731 | DNA | L7-2E09 CDS |
| 732 | DNA | L7-2B10 CDS |
| 733 | DNA | L7-2E10 CDS |
| 734 | DNA | L7-2G10 CDS |
| 735 | DNA | L7-2C11 CDS |
| 736 | DNA | L7-2D11 CDS |
| 737 | DNA | L7-2F11 CDS |
| 738 | DNA | L7-2G11 CDS |
| 739 | DNA | L7-2B12 CDS |
| 740 | DNA | L7-2C12 CDS |
| 741 | DNA | L7-2D12 CDS |
| 742 | DNA | L7-2F12 CDS |
| 743 | DNA | L7-2G12 CDS |
| 744 | DNA | L7-3A01 CDS |
| 745 | DNA | L7-3C01 CDS |
| 746 | DNA | L7-3G01 CDS |
| 747 | DNA | L7-3H01 CDS |
| 748 | DNA | L7-3A02 CDS |
| 749 | DNA | L7-3B02 CDS |
| 750 | DNA | L7-3D02 CDS |
| 751 | DNA | L7-3G02 CDS |
| 752 | DNA | L7-3H02 CDS |
| 753 | DNA | L7-3B03 CDS |
| 754 | DNA | L7-3C03 CDS |
| 755 | DNA | L7-3E03 CDS |
| 756 | DNA | L7-3G03 CDS |
| 757 | DNA | L7-3H03 CDS |
| 758 | DNA | L7-3B04 CDS |
| 759 | DNA | L7-3E04 CDS |
| 760 | DNA | L7-3G04 CDS |
| 761 | DNA | L7-3A05 CDS |
| 762 | DNA | L7-3B05 CDS |
| 763 | DNA | L7-3H05 CDS |
| 764 | DNA | L7-3B06 CDS |
| 765 | DNA | L7-3D06 CDS |
| 766 | DNA | L7-3E06 CDS |
| 767 | DNA | L7-3A07 CDS |
| 768 | DNA | L7-3C07 CDS |
| 769 | DNA | L7-3F07 CDS |
| 770 | DNA | L7-3A08 CDS |
| 771 | DNA | L7-3B08 CDS |
| 772 | DNA | L7-3C08 CDS |
| 773 | DNA | L7-3F08 CDS |
| 774 | DNA | L7-3G08 CDS |
| 775 | DNA | L7-3B09 CDS |
| 776 | DNA | L7-3F09 CDS |
| 777 | DNA | L7-3A10 CDS |
| 778 | DNA | L7-3B10 CDS |
| 779 | DNA | L7-3C10 CDS |
| 780 | DNA | L7-3G10 CDS |
| 781 | DNA | L7-3A11 CDS |
| 782 | DNA | L7-3C11 CDS |
| 783 | DNA | L7-3E11 CDS |
| 784 | DNA | L7-3G11 CDS |
| 785 | DNA | L7-3Al2 CDS |
| 786 | DNA | L7-3B12 CDS |
| 787 | DNA | L7-3C12 CDS |
| 788 | DNA | L7-3E12 CDS |
| 789 | DNA | L7-3F12 CDS |
| 790 | DNA | L7-3G12 CDS |
| 791 | DNA | L7-4A01 CDS |
| 792 | DNA | L7-4A03 CDS |
| 793 | DNA | L7-4A04 CDS |
| 794 | DNA | L7-4A06 CDS |
| 795 | DNA | L7-4A08 CDS |
| 796 | DNA | L7-4A09 CDS |
| 797 | DNA | L7-4A12 CDS |
| 798 | DNA | L7-4B03 CDS |
| 799 | DNA | L7-4B04 CDS |
| 800 | DNA | L7-4B06 CDS |
| 801 | DNA | L7-4B07 CDS |
| 802 | DNA | L7-4C01 CDS |
| 803 | DNA | L7-4C03 CDS |
| 804 | DNA | L7-4C04 CDS |
| 805 | DNA | L7-4C06 CDS |
| 806 | DNA | L7-4C09 CDS |
| 807 | DNA | L7-4C12 CDS |
| 808 | DNA | L7-4D04 CDS |
| 809 | DNA | L7-4D07 CDS |
| 810 | DNA | L7-4D08 CDS |
| 811 | DNA | L7-4D10 CDS |
| 812 | DNA | L7-4D11 CDS |
| 813 | DNA | L7-4E01 CDS |
| 814 | DNA | L7-4E02 CDS |
| 815 | DNA | L7-4E04 CDS |
| 816 | DNA | L7-4E05 CDS |
| 817 | DNA | L7-4E07 CDS |
| 818 | DNA | L7-4E08 CDS |
| 819 | DNA | L6-3A09 CDS |
| 820 | DNA | L7-4C06 (E03) |
| | | CDS |
| 821 | DNA | L10-84(B12) |
| | | CDS |
| 822 | DNA | L13-2-46(D10) |
| | | CDS |
| 823 | DNA | L12-1-10 CDS |
| 824 | DNA | L13-2-23 CDS |
| 825 | DNA | L7-1C3-A5 |
| 826 | DNA | L7-1F8-A11 |
| 827 | DNA | L7-1G6-B2 |
| 828 | DNA | L7-3E3-D1 |
| 829 | DNA | L1-18 CDS |
| 830 | DNA | L1-21 CDS |
| 831 | DNA | L1-25 CDS |
| 832 | DNA | L1-33 CDS |
| 833 | DNA | L1-34 CDS |
| 834 | DNA | L1-36 CDS |
| 835 | DNA | L1-39 CDS |
| 836 | DNA | L1-41 CDS |
| 841 | DNA | Plasmid |
| | | PHP37586A |
| 842 | DNA | Plasmid |
| | | PHP37587A |
| 843 | DNA | Plasmid |
| | | PHP37588A |
| 844 | DNA | Plasmid |
| | | PHP37589A |
| 845 | DNA | Plasmid |
| | | PHP39389A |
| 846 | DNA | Plasmid |
| | | PHP39390A |
| 847 | DNA | Construct |
| | | containing |
| | | artificial |
| | | microRNA |
| 848 | DNA | Tet operator |
| | | sequence |
| 863 | AA | L13-23 |
| 864 | AA | L15-20 |
| 865 | AA | L15-20-M4 |
| 866 | AA | L15-20-M9 |
| 867 | AA | L15-20-M34 |
| 868 | AA | CsL4.2-20 |
| | | having the L17G |
| | | mutation |
| 869 | AA | CsL4.2-15 |
| 870 | AA | CsL4.2-20 |
| 884 | AA | L13-23 having |
| | | the L 17G |
| | | mutation |
| 885 | AA | L15-20 having |
| | | the L 17G |
| | | mutation |
| 886 | AA | L15-20-M4 |
| | | having the L17G |
| | | mutation |
| 887 | AA | L15-20-M9 |
| | | having the L17G |
| | | mutation |
| 888 | AA | L15-20-M34 |
| | | having the L17G |
| | | mutation |
| 889 | AA | CsL4.2-15 |
| | | having the L17G |
| | | mutation |
| 1193 | DNA | L10-11(A04) |
| 1194 | DNA | L10-13(A05) |
| 1195 | DNA | L10-15(A06) |
| 1196 | DNA | L10-30(A09) |
| 1197 | DNA | L10-35(A11) |
| 1198 | DNA | L10-46(B02) |
| 1199 | DNA | L10-47(B03) |
| 1200 | DNA | L10-54(B06) |
| 1201 | DNA | L10-55(B07) |
| 1202 | DNA | L10-59(B08) |
| 1203 | DNA | L10-72(B10) |
| 1204 | DNA | L10-84(B12) |
| 1205 | DNA | L10-90(C02) |
| 1206 | DNA | L11-17(C06) |
| 1207 | DNA | L11-53(C09) |
| 1208 | DNA | L12-1-03 |
| 1209 | DNA | L12-1-06 |
| 1210 | DNA | L12-1-09 |
| 1211 | DNA | L12-1-10 |
| 1212 | DNA | L12-1-11 |
| 1213 | DNA | L12-1-12 |
| 1214 | DNA | L12-1-16 |
| 1215 | DNA | L12-1-17 |
| 1216 | DNA | L12-1-19 |
| 1217 | DNA | L12-1-20 |
| 1218 | DNA | L12-1-21 |
| 1219 | DNA | L12-1-22 |
| 1220 | DNA | L12-2-13 |
| 1221 | DNA | L12-2-14 |
| 1222 | DNA | L12-2-15 |
| 1223 | DNA | L12-2-20 |
| 1224 | DNA | L12-2-22 |
| 1225 | DNA | L12-2-23 |
| 1226 | DNA | L12-2-27 |
| 1227 | DNA | L12-2-33 |
| 1228 | DNA | L12-2-39 |
| 1229 | DNA | L12-2-48 |
| 1230 | DNA | L12-2-49 |
| 1231 | DNA | L12-2-50 |
| 1232 | DNA | L13-1-01 |
| 1233 | DNA | L13-1-02 |
| 1234 | DNA | L13-1-03 |
| 1235 | DNA | L13-1-04 |
| 1236 | DNA | L13-1-05 |
| 1237 | DNA | L13-1-06 |
| 1238 | DNA | L13-1-07 |
| 1239 | DNA | L13-1-08 |
| 1240 | DNA | L13-1-09 |
| 1241 | DNA | L13-1-10 |
| 1242 | DNA | L13-1-11 |
| 1243 | DNA | L13-1-12 |
| 1244 | DNA | L13-1-13 |
| 1245 | DNA | L13-1-14 |
| 1246 | DNA | L13-1-15 |
| 1247 | DNA | L13-1-16 |
| 1248 | DNA | L13-1-17 |
| 1249 | DNA | L13-1-18 |
| 1250 | DNA | L13-1-19 |
| 1251 | DNA | L13-1-20 |
| 1252 | DNA | L13-1-21 |
| 1253 | DNA | L13-1-22 |
| 1254 | DNA | L13-1-23 |
| 1255 | DNA | L13-1-24 |
| 1256 | DNA | L13-1-25 |
| 1257 | DNA | L13-1-26 |
| 1258 | DNA | L13-1-27 |
| 1259 | DNA | L13-1-28 |
| 1260 | DNA | L13-1-29 |
| 1261 | DNA | L13-1-30 |
| 1262 | DNA | L13-1-31 |
| 1263 | DNA | L13-1-32 |
| 1264 | DNA | L13-1-33 |
| 1265 | DNA | L13-1-34 |
| 1266 | DNA | L13-1-35 |
| 1267 | DNA | L13-1-36 |
| 1268 | DNA | L13-1-37 |
| 1269 | DNA | L13-1-38 |
| 1270 | DNA | L13-1-39 |
| 1271 | DNA | L13-1-40 |
| 1272 | DNA | L13-1-41 |
| 1273 | DNA | L13-1-42 |
| 1274 | DNA | L13-1-43 |
| 1275 | DNA | L13-1-44 |
| 1276 | DNA | L13-1-45 |
| 1277 | DNA | L13-1-47 |
| 1278 | DNA | L13-1-48 |
| 1279 | DNA | L13-2-13 |
| 1280 | DNA | L13-2-14 |
| 1281 | DNA | L13-2-15 |
| 1282 | DNA | L13-2-16 |
| 1283 | DNA | L13-2-17 |
| 1284 | DNA | L13-2-18 |
| 1285 | DNA | L13-2-19 |
| 1286 | DNA | L13-2-20 |
| 1287 | DNA | L13-2-21 |
| 1288 | DNA | L13-2-22 |
| 1289 | DNA | L13-2-23 |
| 1290 | DNA | L13-2-24 |
| 1291 | DNA | L13-2-27 |
| 1292 | DNA | L13-2-28 |
| 1293 | DNA | L13-2-29 |
| 1294 | DNA | L13-2-30 |
| 1295 | DNA | L13-2-31 |
| 1296 | DNA | L13-2-32 |
| 1297 | DNA | L13-2-33 |
| 1298 | DNA | L13-2-34 |
| 1299 | DNA | L13-2-35 |
| 1300 | DNA | L13-2-36 |
| 1301 | DNA | L13-2-38 |
| 1302 | DNA | L13-2-39 |
| 1303 | DNA | L13-2-40 |
| 1304 | DNA | L13-2-41 |
| 1305 | DNA | L13-2-42 |
| 1306 | DNA | L13-2-43 |
| 1307 | DNA | L13-2-44 |
| 1308 | DNA | L13-2-45 |
| 1309 | DNA | L13-2-46 |
| 1310 | DNA | L13-2-47 |
| 1311 | DNA | L13-2-48 |
| 1312 | DNA | L13-2-51 |
| 1313 | DNA | L13-2-52 |
| 1314 | DNA | L13-2-53 |
| 1315 | DNA | L13-2-54 |
| 1316 | DNA | L13-2-55 |
| 1317 | DNA | L13-2-56 |
| 1318 | DNA | L13-2-57 |
| 1319 | DNA | L13-2-58 |
| 1320 | DNA | L13-2-59 |
| 1321 | DNA | L13-2-60 |
| 1322 | DNA | L13-2-61 |
| 1323 | DNA | L13-2-62 |
| 1324 | DNA | L13-2-63 |
| 1325 | DNA | L13-2-64 |
| 1326 | DNA | L13-2-65 |
| 1327 | DNA | L13-2-66 |
| 1328 | DNA | L13-2-67 |
| 1329 | DNA | L13-2-68 |
| 1330 | DNA | L13-2-69 |
| 1331 | DNA | L13-2-70 |
| 1332 | DNA | L13-2-71 |
| 1333 | DNA | L13-2-72 |
| 1334 | DNA | L13-2-73 |
| 1335 | DNA | L13-2-74 |
| 1336 | DNA | L13-2-75 |
| 1337 | DNA | L15-01 |
| 1338 | DNA | L15-02 |
| 1339 | DNA | L15-03 |
| 1340 | DNA | L15-04 |
| 1341 | DNA | L15-05 |
| 1342 | DNA | L15-06 |
| 1343 | DNA | L15-07 |
| 1344 | DNA | L15-08 |
| 1345 | DNA | L15-10 |
| 1346 | DNA | L15-11 |
| 1347 | DNA | L15-12 |
| 1348 | DNA | L15-13 |
| 1349 | DNA | L15-14 |
| 1350 | DNA | L15-15 |
| 1351 | DNA | L15-16 |
| 1352 | DNA | L15-17 |
| 1353 | DNA | L15-18 |
| 1354 | DNA | L15-19 |
| 1355 | DNA | L15-20 |
| 1356 | DNA | L15-21 |
| 1357 | DNA | L15-22 |
| 1358 | DNA | L15-23 |
| 1359 | DNA | L15-25 |
| 1360 | DNA | L15-26 |
| 1361 | DNA | L15-27 |
| 1362 | DNA | L15-28 |
| 1363 | DNA | L15-29 |
| 1364 | DNA | L15-30 |
| 1365 | DNA | L15-31 |
| 1366 | DNA | L15-32 |
| 1367 | DNA | L15-33 |
| 1368 | DNA | L15-34 |
| 1369 | DNA | L15-35 |
| 1370 | DNA | L15-36 |
| 1371 | DNA | L15-37 |
| 1372 | DNA | L15-38 |
| 1373 | DNA | L15-39 |
| 1374 | DNA | L15-40 |
| 1375 | DNA | L15-41 |
| 1376 | DNA | L15-42 |
| 1377 | DNA | L15-43 |
| 1378 | DNA | L15-44 |
| 1379 | DNA | L15-45 |
| 1380 | DNA | L15-46 |
| 1381 | AA | L10-11(A04) |
| 1382 | AA | L10-13(A05) |
| 1383 | AA | L10-15(A06) |
| 1384 | AA | L10-30(A09) |
| 1385 | AA | L10-35(A11) |
| 1386 | AA | L10-46(B02) |
| 1387 | AA | L10-47(B03) |
| 1388 | AA | L10-54(B06) |
| 1389 | AA | L10-55(B07) |
| 1390 | AA | L10-59(B08) |
| 1391 | AA | L10-72(B10) |
| 1392 | AA | L10-84(B12) |
| 1393 | AA | L10-90(C02) |
| 1394 | AA | L11-17(C06) |
| 1395 | AA | L11-53(C09) |
| 1396 | AA | L12-1-03 |
| 1397 | AA | L12-1-06 |
| 1398 | AA | L12-1-09 |
| 1399 | AA | L12-1-10 |
| 1400 | AA | L12-1-11 |
| 1401 | AA | L12-1-12 |
| 1402 | AA | L12-1-16 |
| 1403 | AA | L12-1-17 |
| 1404 | AA | L12-1-19 |
| 1405 | AA | L12-1-20 |
| 1406 | AA | L12-1-21 |
| 1407 | AA | L12-1-22 |
| 1408 | AA | L12-2-13 |
| 1409 | AA | L12-2-14 |
| 1410 | AA | L12-2-15 |
| 1411 | AA | L12-2-20 |
| 1412 | AA | L12-2-22 |
| 1413 | AA | L12-2-23 |
| 1414 | AA | L12-2-27 |
| 1415 | AA | L12-2-33 |
| 1416 | AA | L12-2-39 |
| 1417 | AA | L12-2-48 |
| 1418 | AA | L12-2-49 |
| 1419 | AA | L12-2-50 |
| 1420 | AA | L13-1-01 |
| 1421 | AA | L13-1-02 |
| 1422 | AA | L13-1-03 |
| 1423 | AA | L13-1-04 |
| 1424 | AA | L13-1-05 |
| 1425 | AA | L13-1-06 |
| 1426 | AA | L13-1-07 |
| 1427 | AA | L13-1-08 |
| 1428 | AA | L13-1-09 |
| 1429 | AA | L13-1-10 |
| 1430 | AA | L13-1-11 |
| 1431 | AA | L13-1-12 |
| 1432 | AA | L13-1-13 |
| 1433 | AA | L13-1-14 |
| 1434 | AA | L13-1-15 |
| 1435 | AA | L13-1-16 |
| 1436 | AA | L13-1-17 |
| 1437 | AA | L13-1-18 |
| 1438 | AA | L13-1-19 |
| 1439 | AA | L13-1-20 |
| 1440 | AA | L13-1-21 |
| 1441 | AA | L13-1-22 |
| 1442 | AA | L13-1-23 |
| 1443 | AA | L13-1-24 |
| 1444 | AA | L13-1-25 |
| 1445 | AA | L13-1-26 |
| 1446 | AA | L13-1-27 |
| 1447 | AA | L13-1-28 |
| 1448 | AA | L13-1-29 |
| 1449 | AA | L13-1-30 |
| 1450 | AA | L13-1-31 |
| 1451 | AA | L13-1-32 |
| 1452 | AA | L13-1-33 |
| 1453 | AA | L13-1-34 |
| 1454 | AA | L13-1-35 |
| 1455 | AA | L13-1-36 |
| 1456 | AA | L13-1-37 |
| 1457 | AA | L13-1-38 |
| 1458 | AA | L13-1-39 |
| 1459 | AA | L13-1-40 |
| 1460 | AA | L13-1-41 |
| 1461 | AA | L13-1-42 |
| 1462 | AA | L13-1-43 |
| 1463 | AA | L13-1-44 |
| 1464 | AA | L13-1-45 |
| 1465 | AA | L13-1-47 |
| 1466 | AA | L13-1-48 |
| 1467 | AA | L13-2-13 |
| 1468 | AA | L13-2-14 |
| 1469 | AA | L13-2-15 |
| 1470 | AA | L13-2-16 |
| 1471 | AA | L13-2-17 |
| 1472 | AA | L13-2-18 |
| 1473 | AA | L13-2-19 |
| 1474 | AA | L13-2-20 |
| 1475 | AA | L13-2-21 |
| 1476 | AA | L13-2-22 |
| 1477 | AA | L13-2-23 |
| 1478 | AA | L13-2-24 |
| 1479 | AA | L13-2-27 |
| 1480 | AA | L13-2-28 |
| 1481 | AA | L13-2-29 |
| 1482 | AA | L13-2-30 |
| 1483 | AA | L13-2-31 |
| 1484 | AA | L13-2-32 |
| 1485 | AA | L13-2-33 |
| 1486 | AA | L13-2-34 |
| 1487 | AA | L13-2-35 |
| 1488 | AA | L13-2-36 |
| 1489 | AA | L13-2-38 |
| 1490 | AA | L13-2-39 |
| 1491 | AA | L13-2-40 |
| 1492 | AA | L13-2-41 |
| 1493 | AA | L13-2-42 |
| 1494 | AA | L13-2-43 |
| 1495 | AA | L13-2-44 |
| 1496 | AA | L13-2-45 |
| 1497 | AA | L13-2-46 |
| 1498 | AA | L13-2-47 |
| 1499 | AA | L13-2-48 |
| 1500 | AA | L13-2-51 |
| 1501 | AA | L13-2-52 |
| 1502 | AA | L13-2-53 |
| 1503 | AA | L13-2-54 |
| 1504 | AA | L13-2-55 |
| 1505 | AA | L13-2-56 |
| 1506 | AA | L13-2-57 |
| 1507 | AA | L13-2-58 |
| 1508 | AA | L13-2-59 |
| 1509 | AA | L13-2-60 |
| 1510 | AA | L13-2-61 |
| 1511 | AA | L13-2-62 |
| 1512 | AA | L13-2-63 |
| 1513 | AA | L13-2-64 |
| 1514 | AA | L13-2-65 |
| 1515 | AA | L13-2-66 |
| 1516 | AA | L13-2-67 |
| 1517 | AA | L13-2-68 |
| 1518 | AA | L13-2-69 |
| 1519 | AA | L13-2-70 |
| 1520 | AA | L13-2-71 |
| 1521 | AA | L13-2-72 |
| 1522 | AA | L13-2-73 |
| 1523 | AA | L13-2-74 |
| 1524 | AA | L13-2-75 |
| 1525 | AA | L15-01 |
| 1526 | AA | L15-02 |
| 1527 | AA | L15-03 |
| 1528 | AA | L15-04 |
| 1529 | AA | L15-05 |
| 1530 | AA | L15-06 |
| 1531 | AA | L15-07 |
| 1532 | AA | L15-08 |
| 1533 | AA | L15-10 |
| 1534 | AA | L15-11 |
| 1535 | AA | L15-12 |
| 1536 | AA | L15-13 |
| 1537 | AA | L15-14 |
| 1538 | AA | L15-15 |
| 1539 | AA | L15-16 |
| 1540 | AA | L15-17 |
| 1541 | AA | L15-18 |
| 1542 | AA | L15-19 |
| 1543 | AA | L15-20 |
| 1544 | AA | L15-21 |
| 1545 | AA | L15-22 |
| 1546 | AA | L15-23 |
| 1547 | AA | L15-25 |
| 1548 | AA | L15-26 |
| 1549 | AA | L15-27 |
| 1550 | AA | L15-28 |
| 1551 | AA | L15-29 |
| 1552 | AA | L15-30 |
| 1553 | AA | L15-31 |
| 1554 | AA | L15-32 |
| 1555 | AA | L15-33 |
| 1556 | AA | L15-34 |
| 1557 | AA | L15-35 |
| 1558 | AA | L15-36 |
| 1559 | AA | L15-37 |
| 1560 | AA | L15-38 |
| 1561 | AA | L15-39 |
| 1562 | AA | L15-40 |
| 1563 | AA | L15-41 |
| 1564 | AA | L15-42 |
| 1565 | AA | L15-43 |
| 1566 | AA | L15-44 |
| 1567 | AA | L15-45 |
| 1568 | AA | L15-46 |
| 1949 | DNA | L8-1A03 |
| 1950 | DNA | L8-1A04 |
| 1951 | DNA | L8-1A05 |
| 1952 | DNA | L8-1A06 |
| 1953 | DNA | L8-1B12 |
| 1954 | DNA | L8-1C02 |
| 1955 | DNA | L8-1C09 |
| 1956 | DNA | L8-1D03 |
| 1957 | DNA | L8-1D11 |
| 1958 | DNA | L8-1E02 |
| 1959 | DNA | L8-1E04 |
| 1960 | DNA | L8-2A08 |
| 1961 | DNA | L8-2B05 |
| 1962 | DNA | L8-2F04 |
| 1963 | DNA | L8-2F10 |
| 1964 | DNA | L8-2F12 |
| 1965 | DNA | L8-2H01 |
| 1966 | DNA | L8-3A04 |
| 1967 | DNA | L8-3A05 |
| 1968 | DNA | L8-3A06 |
| 1969 | DNA | L8-3A07 |
| 1970 | DNA | L8-3A10 |
| 1971 | DNA | L8-3A12 |
| 1972 | DNA | L8-3B02 |
| 1973 | DNA | L8-3B03 |
| 1974 | DNA | L8-3B05 |
| 1975 | DNA | L8-3B08 |
| 1976 | DNA | L8-3B09 |
| 1977 | DNA | L8-3D03 |
| 1978 | DNA | L8-3D04 |
| 1979 | DNA | L8-3D12 |
| 1980 | DNA | L8-3E05 |
| 1981 | DNA | L8-3E09 |
| 1982 | DNA | L8-3F01 |
| 1983 | DNA | L8-3F02 |
| 1984 | DNA | L8-3F06 |
| 1985 | DNA | L8-3F08 |
| 1986 | DNA | L8-3F09 |
| 1987 | DNA | CsL3-1A07 |
| 1988 | DNA | CsL3-1B04 |
| 1989 | DNA | CsL3-1B05 |
| 1990 | DNA | CsL3-1B11 |
| 1991 | DNA | CsL3-1C01 |
| 1992 | DNA | CsL3-1C12 |
| 1993 | DNA | CsL3-2A01 |
| 1994 | DNA | CsL3-2B06 |
| 1995 | DNA | CsL3-2B09 |
| 1996 | DNA | CsL3-2B12 |
| 1997 | DNA | CsL3-2D02 |
| 1998 | DNA | CsL3-2D10 |
| 1999 | DNA | CsL3-2D11 |
| 2000 | DNA | CsL3-2D12 |
| 2001 | DNA | CsL3-2E07 |
| 2002 | DNA | CsL3-2E08 |
| 2003 | DNA | CsL3-2E09 |
| 2004 | DNA | CsL3-2E10 |
| 2005 | DNA | CsL3-2E11 |
| 2006 | DNA | CsL3-2E12 |
| 2007 | DNA | CsL3-MTZ2 |
| 2008 | DNA | CsL3-MTZ3 |
| 2009 | DNA | CsL3-MTZ4 |
| 2010 | DNA | CsL3-MTZ5 |
| 2011 | DNA | CsL4.2-01 |
| 2012 | DNA | CsL4.2-04 |
| 2013 | DNA | CsL4.2-07 |
| 2014 | DNA | CsL4.2-08 |
| 2015 | DNA | CsL4.2-11 |
| 2016 | DNA | CsL4.2-12 |
| 2017 | DNA | CsL4.2-15 |
| 2018 | DNA | CsL4.2-16 |
| 2019 | DNA | CsL4.2-17 |
| 2020 | DNA | CsL4.2-18 |
| 2021 | DNA | CsL4.2-20 |
| 2022 | DNA | CsL4.2-21 |
| 2023 | DNA | CsL4.2-22 |
| 2024 | DNA | CsL4.2-23 |
| 2025 | DNA | CsL4.2-24 |
| 2026 | DNA | CsL4.2-26 |
| 2027 | DNA | CsL4.2-27 |
| 2028 | DNA | CsL4.2-28 |
| 2029 | DNA | CsL4.2-30 |
| 2030 | AA | L8-1A03 |
| 2031 | AA | L8-1A04 |
| 2032 | AA | L8-1A05 |
| 2033 | AA | L8-1A06 |
| 2034 | AA | L8-1B12 |
| 2035 | AA | L8-1C02 |
| 2036 | AA | L8-1C09 |
| 2037 | AA | L8-1D03 |
| 2038 | AA | L8-1D11 |
| 2039 | AA | L8-1E02 |
| 2040 | AA | L8-1E04 |
| 2041 | AA | L8-2A08 |
| 2042 | AA | L8-2B05 |
| 2043 | AA | L8-2F04 |
| 2044 | AA | L8-2F10 |
| 2045 | AA | L8-2F12 |
| 2046 | AA | L8-2H01 |
| 2047 | AA | L8-3A04 |
| 2048 | AA | L8-3A05 |
| 2049 | AA | L8-3A06 |
| 2050 | AA | L8-3A07 |
| 2051 | AA | L8-3A10 |
| 2052 | AA | L8-3A22 |
| 2053 | AA | L8-3B02 |
| 2054 | AA | L8-3B03 |
| 2055 | AA | L8-3B05 |
| 2056 | AA | L8-3B08 |
| 2057 | AA | L8-3B09 |
| 2058 | AA | L8-3D03 |
| 2059 | AA | L8-3D04 |
| 2060 | AA | L8-3D12 |
| 2061 | AA | L8-3E05 |
| 2062 | AA | L8-3E09 |
| 2063 | AA | L8-3F01 |
| 2064 | AA | L8-3F02 |
| 2065 | AA | L8-3F06 |
| 2066 | AA | L8-3F08 |
| 2067 | AA | L8-3F09 |
| 2068 | AA | CsL3-1A07 |
| 2069 | AA | CsL3-1B04 |
| 2070 | AA | CsL3-1B05 |
| 2071 | AA | CsL3-1B11 |
| 2072 | AA | CsL3-1C01 |
| 2073 | AA | CsL3-1C12 |
| 2074 | AA | CsL3-2A01 |
| 2075 | AA | CsL3-2B06 |
| 2076 | AA | CsL3-2B09 |
| 2077 | AA | CsL3-2B12 |
| 2078 | AA | CsL3-2D02 |
| 2079 | AA | CsL3-2D10 |
| 2080 | AA | CsL3-2D11 |
| 2081 | AA | CsL3-2D12 |
| 2082 | AA | CsL3-2E07 |
| 2083 | AA | CsL3-2E08 |
| 2084 | AA | CsL3-2E09 |
| 2085 | AA | CsL3-2E10 |
| 2086 | AA | CsL3-2E11 |
| 2087 | AA | CsL3-2E12 |
| 2088 | AA | CsL3-MTZ2 |
| 2089 | AA | CsL3-MTZ3 |
| 2090 | AA | CsL3-MTZ4 |
| 2091 | AA | CsL3-5 |
| 2092 | AA | CsL4.2-01 |
| 2093 | AA | CsL4.2-04 |
| 2094 | AA | CsL4.2-07 |
| 2095 | AA | CsL4.2-08 |
| 2096 | AA | CsL4.2-11 |
| 2097 | AA | CsL4.2-12 |
| 2098 | AA | CsL4.2-15 |
| 2099 | AA | CsL4.2-16 |
| 2100 | AA | CsL4.2-17 |
| 2101 | AA | CsL4.2-18 |
| 2102 | AA | CsL4.2-20 |
| 2103 | AA | CsL4.2-21 |
| 2104 | AA | CsL4.2-22 |
| 2105 | AA | CsL4.2-23 |
| 2106 | AA | CsL4.2-24 |
| 2107 | AA | CsL4.2-26 |
| 2108 | AA | CsL4.2-27 |
| 2109 | AA | CsL4.2-28 |
| 2110 | AA | CsL4.2-30 |
| 2111 | DNA | pHD2033-2036 |
| 2112 | DNA | pHD2037-2040 |

The following examples are provided to illustrate some embodiments of the invention, but should not be construed as defining or otherwise limiting any aspect, embodiment, element or any combinations thereof. Modifications of any aspect, embodiment, element or any combinations thereof are apparent to a person of skill in the art.

### EXPERIMENTAL

Chemical based control of transcription in plants with sulfonylurea (SU) herbicides via a modified tet-repressor based mechanism has been demonstrated (US20110294216). Although the strategy relies on repression/de-repression of fully functional promoters having embedded tet operator sequences (Gatz 1988; Frohberg 1991; Gatz 1992; Yao 1998), the mechanism could be modified to create a SU controlled transcriptional activator acting on a minimal promoter with upstream tet operators (Gossen 1995; Schonig 2002). However, as an alternative to transcriptional regulation, it is possible the level of target protein itself can be modulated directly through ligand-dependent stabilization (Johnson 1995, Banaszynski 2006, Lampson 2006, Iwamoto 2010). This would have the advantages of reducing genetic complexity to one expression cassette instead of two (transcriptional regulation requires one for the target gene and one for the transcriptional activator/repressor) and possibly enabling quicker response to ligand as both transcription and translation would have already reached steady state. The promoter driving expression of the destabilized protein could be constitutive, spatio-temporal specific, or inducible. Accumulation of the target gene product in any cell type would be dependent on the presence of the stabilizing ligand.

Chemical regulation of target protein accumulation has thus far been accomplished thru fusion to an established ligand-gated stability domain. This leads to destruction of the fused target protein in the absence of ligand in vivo. A potential drawback to this strategy is that in some cases the target protein will not perform well as a protein fusion even after stabilization. However, this could be circumvented by creating an intein whose stability is chemically regulated by fusion to a ligand-gated stability domain. The resulting intein would then be inserted into any polypeptide sequence of interest to create a destabilized pro-target protein. Upon ligand exposure the target::intein::target protein would accumulate and splicing would release fully mature target protein. Ligand gated intein function has been established in other laboratories (Mootz and Muir 2002; Buskirk et al 2004).

To further enhance regulation, protein and transcriptional switch mechanisms could be combined. As these would be orthogonal methods combining them should lead to synergy. In this regard it is anticipated that the current SU regulated repressor can be modified to create a transcriptional activator whose accumulation is self-regulated by cognate ligand. Observations by Lai et al. (2010) indicate that this may be possible since some reverse TetR transcriptional activators are indeed unstable and subject to proteasomal degradation in the absence of ligand. Even further improvement in regulation can be accomplished by having a SuR negatively regulating expression of a SU dependent activator as well as the target promoter. This would require the regulated promoter to have tet operator sequences located strategically for both repression and activation functionality and the presence of both repressor and activator proteins. Such additional steps may be necessary to enable control of very active gene products that require extremely low basal expression yet need to be significantly induced upon ligand exposure.

We have undertaken a study of our sulfonylurea repressors (SuR's) to determine if they can be modified to selectively accumulate in vivo in the presence of SU herbicides ethametsulfuron-methyl and chlorsulfuron. It has been determined that various mutations of TetR lead to decreased protease resistance of the purified proteins in an in vitro assay and that addition of the tetracycline analog ‘anhydrotetracycline’ can lead to improved protease resistance (Reichheld 2006, Resch 2008, Reichheld 2009). As a result of these findings Reichheld and Davidson (2006) indicated that an undisclosed mutated form of TetR was conditionally stabilized in yeast following tetracycline application (data not shown: discussion section) and that this property could be exploited to conditionally stabilize fusion partners for biotechnology applications. Also disclosed is that so called ‘reverse Tet repressors’, tend to be unstable and can be partially rescued with inducer. Structural studies of an L17G substitution in the DNA binding domain (DBD) of a chimeric TetR-BD that requires tetracycline as a co-repressor reveals a ligand dependent disorder/order shift (Resch et al. 2008). An in vivo study of various reverse repressors used to control gene expression in mammalian cells revealed their ubiquitin gated stability was greatly influenced by the presence of doxycycline (Lai et al. 2010). In contrast to the above examples, our proteins do not bind to tetracycline or anhydrotetracycline and the sequences are divergent thus it was not known if the published ‘destabilizing mutations’ would lead to destabilization of the SU repressors and if so whether herbicide addition could rescue stability. To test this concept, chemical dependent protein accumulation of various mutant ethametsulfuron repressors (EsR's) and chlorsulfuron repressors (CsR's) fused to AcGFP with and without potential destabilizing mutations in the DNA binding domain have been surveyed. We have found that both EsR and CsR GFP fusions with the DBD mutations show vastly increased green fluorescence in both yeast and plants when cognate ligand is present. This indicates that a protein switch mechanism based on the SuR scaffold has been developed and could be extended for use in many eukaryotic organisms.

### Example 1

### Ligand Enhanced TetR Fusion Protein Accumulation in Yeast

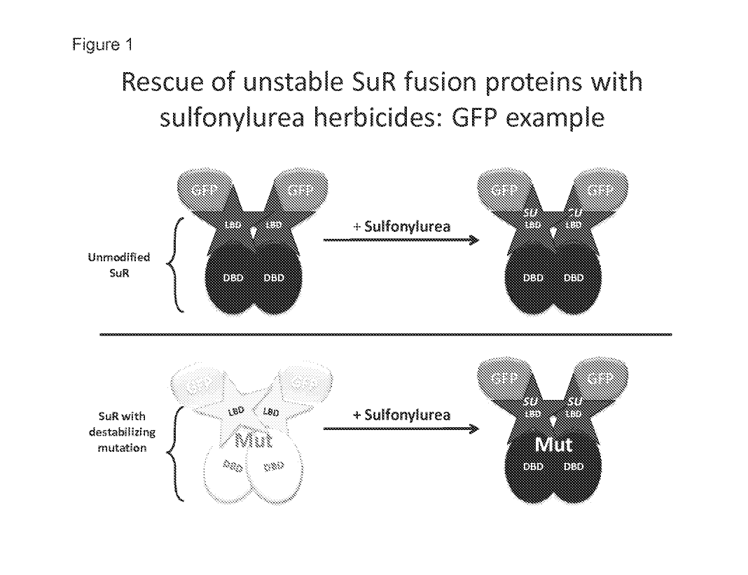

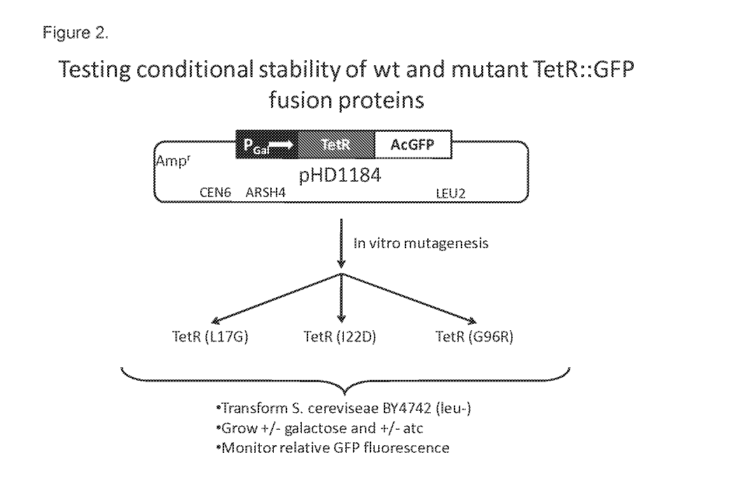

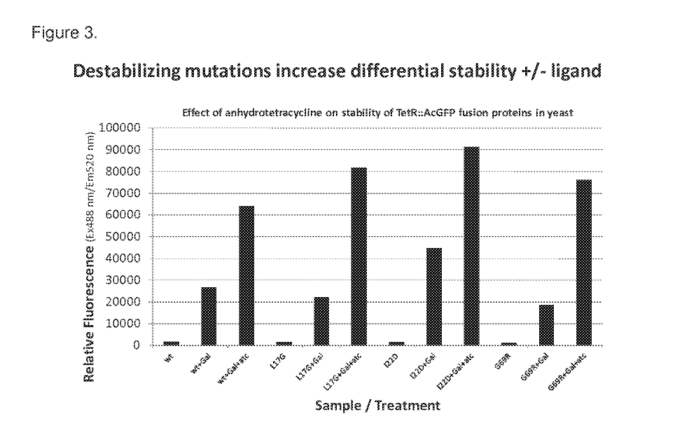

Three mutations in TetR shown to physically destabilize purified protein in the absence of inducer yet be partially suppressed by addition of atc were chosen for this study. Two of the mutations, L17G and G96R (Scholz et al. 2004), were shown to convert TetR into a co-repressor with cognate ligand atc. The third mutation, 122D (Reichheld and Davidson 2006), is a constitutive mutation in the presence or absence of ligand. Both L17G and I22D lie in the DNA binding domain (DBD) whereas G96R is in alpha helix 6 within the ligand binding domain (LBD). To test the effect of these mutations for ligand gated stability a GFP destabilization/re-stabilization assay (FIG. 1) was created. To do this a fusion between the coding regions of TetR B (Wray et al. 1981) and AcGFP (Gurskaya et al. 2003) by PCR amplifying the TetR region from plasmid pVER7568 using primers REPS' and TetR::AcGFP Rev and the AcGFP coding region from plasmid pHD1010 with primers TetR::AcGFP For and AcGFP3′ (Table 2) was created. The PCR products were then combined and subjected to overlap extension PCR using primers REPS' and AcGFP3′. The resulting full length PCR fusion product was then cloned into the Galactose inducible yeast expression vector p415GAL (ATCC#87330) as an XbaI/HindIII fragment. The resulting vector, pHD1184 (FIG. 2), was then subjected to in vitro mutagenesis (Quick Change mutagenesis—Stratagene) with the primers listed in Table 3 to generate pHD2012 [pGAL-TetR(L17G)], pHD2013 [pGAL-TetR(I22D)], and pHD2014 [pGAL-TetR(G96R)]. Each of these vectors were then transformed into S. cereviseae BY4742 (leu-, his-, ade-) and plated onto leu-knockout medium to select for LEU+ colonies. The transformed yeast strains were then grown overnight in minimal broth with ade, his, and 2% glucose and then subcultured into 2 ml of minimal media containing ade, his, and either 2% glucose, 2% galactose, or 2% galactose+10 uM anhydrotetracycline (atc). Following 6 hrs of growth 1 ml of cells were then centrifuged, washed in an equal volume of 1.2 M sorbitol and then resuspended in 250 ul of 1.2 M sorbitol. 100 ul aliquots of resuspended cells were placed into clear bottom black 96-well plates and their fluorescence determined with a Typhoon Laser Image Scanner (GE: emission at 488 nm and excitation at 520 nm). The data shown in FIG. 3 reveal that L17G and G96R mutations have a significant negative impact the accumulation of GFP compared to wt TetR. Interestingly, addition of atc to the medium greatly increased the relative GFP fluorescence in all samples. Thus it is likely that atc is improving the folding efficiency and/or overall stability of the fusion proteins.

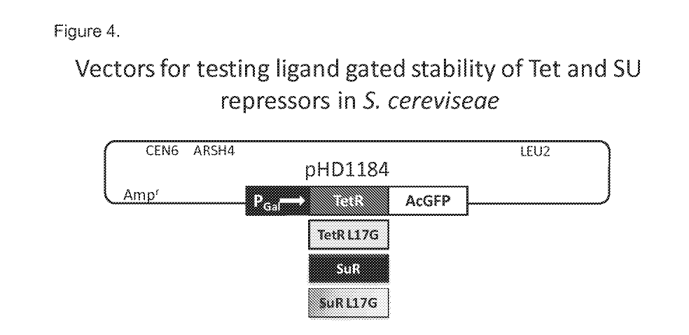

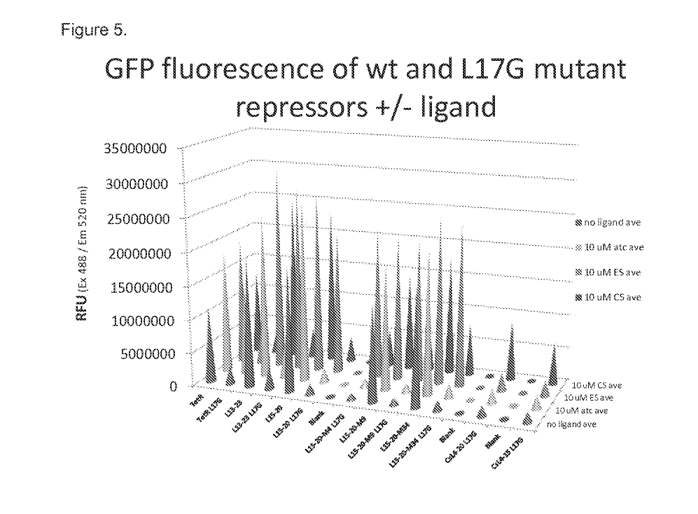

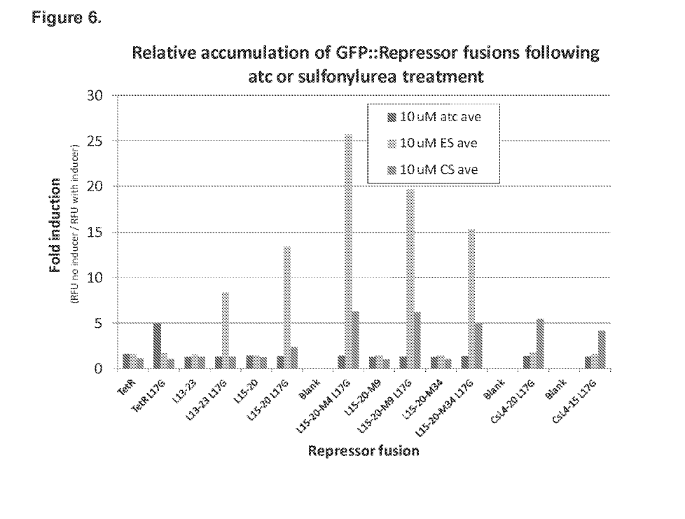

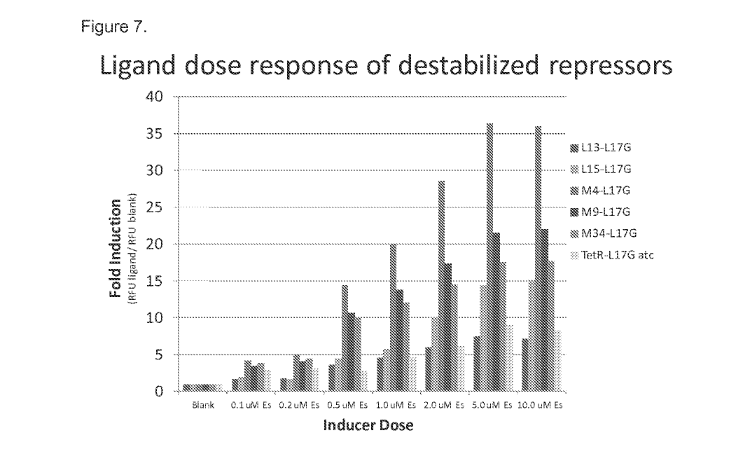

Next, we wanted to determine if a similar ligand enhanced protein accumulation effect would translate to our SU repressor backbones. While the shuffled SU repressors have the same DNA binding domain as TetR B their ligand binding domains are greater than 15% different. Given the number of changes to the parent sequence and the 100% change in ligand preference it was not clear if they would behave in a similar manner. To test this concept, the ligand binding domains from wt and L17G TetR were substituted with EsR hits L13-23, L15-20, L15-20-M4, L15-20-M9, L15-20-M34 and CsR hits CsL4.2-15 and CsL4.2-20. This was done by PCR amplifying the above coding regions with primers REPS' and EsR(L3-23) Rev, EsR(L15-20) Rev, or CsR(L4-20) Rev (Table 2), digesting each PCR product with StuI/BamHI and cloning each product into StuI/BamHI digested backbone fragments of pHD1184 and pHD2012 to give both wt and L17G mutant DNA binding domain combinations, respectively for most of the SuR's (schematic in FIG. 4). The resulting vectors (Table 4) were then transformed into S. cereviseae BY4742 as for pHD1184 (above). Each strain was then grown overnight in YPD medium and the cultures arrayed in 96-well format such that there were four repeats of every strain per plate. The array was then stamped onto 40 ml DOBA agar supplemented with 2% galactose, 0.025% casamino acids, and either 10 uM atc, ethametsulfuron, chlorsulfuron or no addition as the control. The plates were grown two days at 30° C. and imaged using a Typhoon laser scanning imager (GE) with excitation and emission set at 488 and 520 nm respectively. The data (FIG. 5) show that ethametsulfuron repressors (EsR's) are more sensitive to destabilization from the introduced L17G mutation than TetR (compare wt vs L17G for each repressor in absence of ligand) and that the destabilized EsR::GFP fusion proteins respond in a robust manner to addition of Es such that they gain back nearly all the GFP fluorescence lost thru the mutation. Comparison of fold difference in GFP fluorescence between no ligand and 10 uM ligand for each of the L17G mutants (FIG. 6) show that the EsR::GFP fusions respond much more intensely to ligand than the TetR::GFP fusion. In a second experiment (using the same base medium, growth conditions, and data capture mechanism) the ligand sensitivity of the destabilized fusion proteins was examine using a dose response series from 0.1 uM to 10 uM (FIG. 7). The results show that all samples respond weakly at 0.1 uM and that the TetR derivative gives a ˜10× response at 5-10 uM atc whereas many of the EsR derivatives are even more responsive at the 0.5 uM Es dose. This indicates that the destabilized EsR::GFP hits are at least ten-fold more sensitive to ligand-gated re-stabilization than TetR. While the ligand response results for the EsR fusions were dramatic, those for the CsR fusions (CsL4-15 and CsL4-20) were only modest (FIGS. 6 and 7). At 10 uM chlorsulfuron (Cs) both CsR clones tested gave a ˜5× increase in GFP intensity which is up to 5× less than that for the best EsR clones and more equivalent to that seen for destabilized TetR::GFP. Interestingly some of the EsR clones responded significantly to Cs (˜6× increase in fluorescence). This is not surprising since it is known that cross reactivity occurs in these clones to Cs both in genetic and biochemical assays. Overall, these data indicate that stability of all SuR::GFP fusions responds to addition of SU ligands.

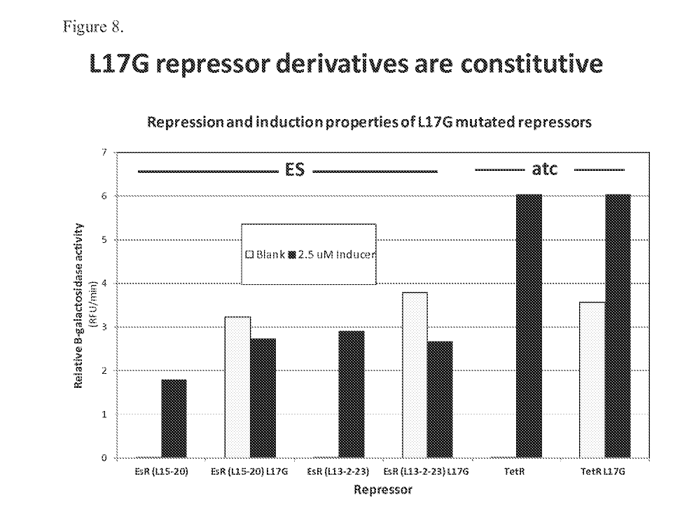

As the L17G mutation performed very well at differential stabilization of subject fusion proteins we sought to determine if this lesion imparted reverse repressor activity onto SuR the same as for TetR (Resch, M. et al. (2008) Nucl. Acids Res. 36:4391-4401). To test this possibility we mutated wt DBD regions of each repressor in the context of the E. coli pBAD expression vector system using oligonucleotides ‘TetR-L17G top’ (Seq ID 878) and ‘TetR-L17G bottom’ (Seq ID 879). After confirming mutations by DNA sequencing each clone was introduced into E. coli strain KM3 and B-galactosidase assays performed. Results show that none of the repressors including TetR exhibit reverse repression activity i.e. constitutive expression in the absence and repression in the presence and of inducer (FIG. 8). The lack of reverse repression activity for the L17G version of TetR(B) studied here relative to the published data for TetR(BD) indicates the lack of predictable effects from similar mutations in different backbones of the same repressor family.

### Example 2

### Sulfonylurea Dependent Protein Accumulation in Planta

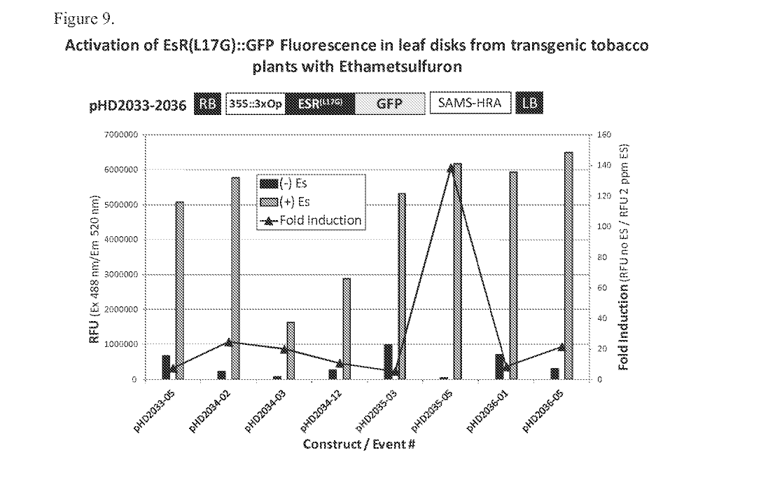

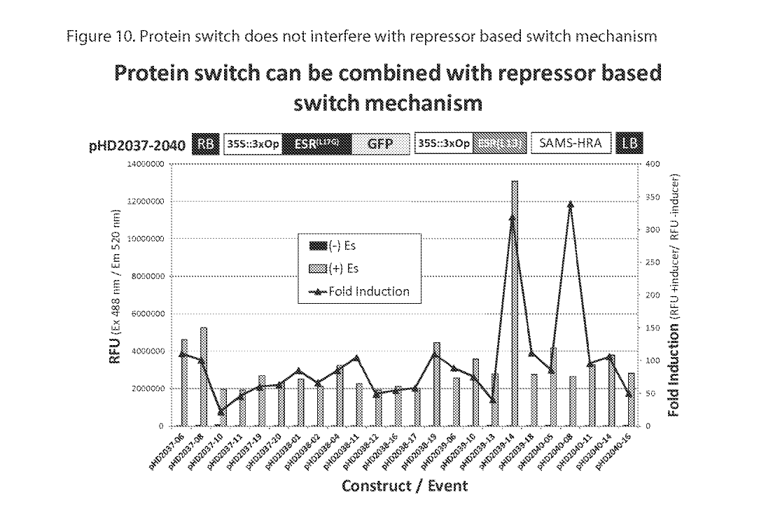

To determine the effect of the L17G mutation on switchable protein stability in planta two series of vectors were constructed. Repressor::GFP fusions for L13-23, L15-20, L15-20-M4, and L15-20-M9 from each of the yeast vectors (above) were subcloned into a repressible plant expression entry clone pVER7581 NcoI to Asp718 to create plasmids pHD2029, pHD2030, pHD2031 and pHD2032, respectively. Each of these entry clones were then assembled into T-DNA vectors using T-DNA destination vector PHP39852, HRA containing sulfonylurea selectable marker entry vector pVER7573, and either with a blank entry clone or entry clone pVER7373 containing an auto-repressible L13-23 repressor cassette. The resulting eight vectors enable testing of the SU dependent protein stability switch by itself (pHD2033 thru pHD2036) and in combination with the transcriptional switch (pHD2036 thru pHD2040). These vectors were transformed into A. tumefaciens EHA105, co-cultivated with tobacco, and tissue selected on 50 ppb imazapyr and herbicide resistant/GFP(−) shoots regenerated into whole tobacco plants. Leaf disk samples were then tested for induction in 48-well microtiter array containing 200 ul of water with or without 2 ppm Ethametsulfuron. Leaf disks were incubated for three days in a Percival incubator set at 25° C. and then imaged with a Typhoon laser scanning imager (GE) as was done for the yeast cultures (above). Those events showing inducibility were tested for copy number by qPCR. Induction of GFP fluorescence in leaf disks of single copy events is shown in FIGS. 9 and 10. Results show that all repressor::GFP fusion proteins resulting from constructs pHD2033 thru pHD2036 respond to Ethametsulfuron treatment similar to what was seen in yeast: ˜5-20 fold enhanced fluorescence. When these repressor::GFP fusions were tested with a functional repressor (constructs pHD2037 thru pHD2040) there was greater control of expression due to repression of transcription in addition to protein stability (FIG. 10). Functional repression exhibited by these latter vectors/events indicates that the destabilized repressor does not cause trans-degradation of wt repressor or malfunction of its DNA binding capacity thru heterodimerization.

### BIBLIOGRAPHY

Gatz et al. (1988) Proc. Natl. Acad. Sci. USA. 85: 1394-1397.
Gatz et al. (1992) The Plant Journal 2: 397-404
Frohberg et al. (1991) Proc. Natl. Acad. Sci. USA. 88: 10470-10474.
Gossen et al. (1995) Science 268, 1766-1769.
Kai et al. (2002) Nucleic Acids Research. 30: e134
Yao et al. (1998) Human Gene Therapy 9:1939-1950
Buskirk et al. (2004) PNAS vol. 101 (29): 10505-10510
Mootz et al. (2002) J. Am. Chem. Soc., 124 (31), pp 9044-9045
Johnson, J A et al. (1995) J Biol. Chem. 270:8172-8178.
Banaszynski et al. (2006) Cell 126:995-1004.
Lai et al. (2004)J Gene Med 6: 1403-1413.
Lampson et al. (2006) Cell 126:827-829.
Iwamoto, M. et al. (2010) Chemistry and Biology 17:981-988.
Reichheld et al. (2006)J Mol. Biol. 361:382-389.
Reichheld et al. (2009) PNAS 106:22263-22268.
Resch, M. et al. (2008) Nucl. Acids Res. 36:4391-4401.
Scholz et al. (2004) Molecular Microbiology 2004. 53: 777-789.
Wray et al. (1981)J Bacteriol. 147, 297-304
Gurskaya et al. (2003) Biochem. J. 373: 403-408

TABLE 2

| Name | Description | Oligo Sequence | SEQ ID NO |
|---|---|---|---|
| REP5′ | adds Xba/Nco to 5′ end | ACACATCTAGAAACCATGGCCAGAC | 871 |
| | of all plant optimized | TCGACAAGAG | |
| | repressors | | |
| AcGFP3′ | adds Asp and Hind3 to | TGTGTAAGCTTGTTGGTACCTCACTT | 872 |
| | 3′ of AcGFP | GTACAGCTCATCCATGC | |
| TetR::Ac | top strand primer to | CTGAAGTGTGAAAGTGGGTCTGGAT | 873 |
| GFP For | create fusion between | CCGTGAGCAAGGGCGCCGAGCTG | |
| | TetR and AcGFP. Adds | | |
| | BamH1 site at the | | |
| | junction | | |
| TetR::Ac | bottom strand primer to | CAGCTCGGCGCCCTTGCTCACGGATC | 874 |
| GFP Rev | create fusion between | CAGACCCACTTTCACACTTCAG | |
| | TetR and AcGFP. Adds | | |
| | BamH1 site at the | | |
| | junction | | |
| EsR(L13- | Primes L13-23 and adds | GCTCACGGATCCAGATCCACTTTCAC | 875 |
| 23) Rev | BamH1 site to 3′ for | ACTTCAG | |
| | cloning into AcGFP | | |
| | fusion cassettes | | |
| EsR(L15- | Primes L15-20 and adds | GCTCACGGATCCAGACCCACTTTCGG | 876 |
| 20) Rev | BamH1 site to 3′ for | CCTTCAG | |
| | cloning into AcGFP | | |
| | fusion cassettes | | |
| CsR(L4- | Primes CsR(L4-20) and | GCTCACGGATCCAGACCCACTTTCTC | 877 |
| 20) Rev | adds BamH1 site to 3′ | TCTTCAG | |
| | for cloning into AcGFP | | |
| | fusion cassettes | | |

TABLE 3

| | | | SEQ |
|---|---|---|---|
| | | | ID |
| Name | Description | Oligo Sequence | NO |
| TetR- | TetR-AcGFP L17G | CGATTCCGACCTCGTTCCCCA | 878 |
| L17G | bottom strand | GCTCCAGTGCGCTGTTG | |
| Bottom | mutagenesis primer | | |
| TetR- | TetR-AcGFP L17G | CAACAGCGCACTGGAGCTGGG | 879 |
| L17G | top strand | GAACGAGGTCGGAATCG | |
| Top | mutagenesis primer | | |
| TetR- | TetR-AcGFP G96R | CTAGGTGGACCTTGGCTCGAT | 880 |
| G96R | bottom strand | CACGGTGACTGAGC | |
| Bottom | mutagenesis primer | | |
| TetR- | TetR-AcGFP G96R | GCTCAGTCACCGTGATCGAGC | 881 |
| G96R | top strand | CAAGGTCCACCTAG | |
| Top | mutagenesis primer | | |
| TetR- | TetR-AcGFP I22D | GCTGAACGAGGTCGGAGACGA | 882 |
| I22D | top stand | AGGCCTCACAACCCG | |
| Top | mutagenesis primer | | |
| TetR- | TetR-AcGFP I22D | CGGGTTGTGAGGCCTTCGTCT | 883 |
| I22D | bottom strand | CCGACCTCGTTCAGC | |
| Bottom | mutagenesis primer | | |

TABLE 4

| Name | Description |
|---|---|
| pHD1184 | Pgal-TetR::AcGFP/LEU2/AmpR |
| pHD2012 | Pgal-TetR(L17G)::AcGFP/LEU2/AmpR |
| pHD2013 | Pgal-TetR(I22D)::AcGFP/LEU2/AmpR |
| pHD2014 | Pgal-TetR(G96R)::AcGFP/LEU2/AmpR |
| pHD2015 | Pgal-EsR(L13-23)::AcGFP/LEU2/AmpR |
| pHD2016 | Pgal-EsR(L13-23-L17G)::AcGFP/LEU2/AmpR |
| pHD2017 | Pgal-EsR(L15-20)::AcGFP/LEU2/AmpR |
| pHD2018 | Pgal-EsR(L15-20-L17G)::AcGFP/LEU2/AmpR |
| pHD2019 | Pgal-EsR(L15-20-M4)::AcGFP/LEU2/AmpR |
| pHD2020 | Pgal-EsR(L15-20-M4-L17G)::AcGFP/LEU2/AmpR |
| pHD2021 | Pgal-EsR(L15-20-M9)::AcGFP/LEU2/AmpR |
| pHD2022 | Pgal-EsR(L15-20-M9-L17G)::AcGFP/LEU2/AmpR |
| pHD2023 | Pgal-EsR(L15-20-M34)::AcGFP/LEU2/AmpR |
| pHD2024 | Pgal-EsR(L15-20-M34-L17G)::AcGFP/LEU2/AmpR |
| pHD2025 | Pgal-CsR(4.2-15)::AcGFP/LEU2/AmpR |
| pHD2026 | Pgal-CsR(4.2-15-L17G)::AcGFP/LEU2/AmpR |
| pHD2027 | Pgal-CsR(4.2-20)::AcGFP/LEU2/AmpR |
| pHD2028 | Pgal-CsR(4.2-20-L17G)::AcGFP/LEU2/AmpR |

### Example 3

### Further Shuffling for Improved Ethametsulfuron Repressor Variants

### A. Fourth Round Shuffling

Fourth round shuffling was designed from phylogenetic alignments of TetR(B) homologues at 13 previously untested positions in addition to retesting selected substitutions at 23 previously shuffled positions. Also, the six cysteine residues aligning to wt TetR were varied with phylogenetically available diversity. This brought the total number of shuffled residues to 42. To screen this diversity two libraries, L10 and L11, were constructed (Table 5). As was done for L4 the diversity was titrated into the synthetic oligonucleotide mixture along with oligonucleotides representing parent clone L7-A11 to reduce the complexity of each individual clone (Table 6A-C).

TABLE 5
Diversity summary for libraries L10 thru L15.

| Residue | TetR (B) | | | | | |
|---|---|---|---|---|---|---|
| position | Residue | L10 | L11 | L12 | L13 | L15 |
| 55 | L | M | M | M | M | |
| 57 | I | IF | — | IF | — | |
| 60 | L | — | — | — | LF | |
| 61 | D | — | NED | — | — | |
| 62 | R | PR | — | PR | — | P [symbol] |
| 64 | H | A | A | A | ADEKR (SEQ ID | [symbol] QRT (SEQ |
| | | | | | NO: 2115) | ID NO: 2116) |
| 65 | T | [symbol] PT | — | [symbol] PT | — | IT |
| 66 | H | — | HQY | — | — | |
| 67 | F | LFY | Y | [symbol] F | Y | |
| 68 | C | LSC | LSC | LC | LC | |
| 69 | P | P [symbol] | — | L | — | |
| 71 | E | — | VE | — | — | |
| 73 | E | [symbol] E | — | AE | — | |
| 77 | D | — | DN | DNQ | — | DN |
| 82 | N | N [symbol] | — | [symbol] K | [symbol] [symbol] N | [symbol] N [symbol] |
| | | | | | | (SEQ ID |
| | | | | | | NO: 2117) |
| 86 | F | M | M | M | [symbol] R | |
| 88 | C | RNC | RNC | N | N | |
| 99 | V | [symbol] A | — | — | — | |
| 100 | H | C | C | C | C [symbol] | AC |
| 104 | R | G | GA | G | G | |
| 105 | P | FL | IVW [symbol] (SEQ | F | F | |
| | | | ID NO: 2118) | | | |
| 108 | K | Q | [symbol] N | Q | Q | [symbol] RK |
| 109 | Q | QN | — | — | — | |
| 113 | L | AT | LVI [symbol] | A | AM | AMQS (SEQ |
| | | | | | | ID NO: 2119) |
| 114 | E | — | — | — | — | |
| 116 | Q | SR | MQS | SRQ | [symbol] W | [symbol] W (SEQ |
| | | | | | | ID NO: 2120) |
| 121 | C | TC | [symbol] C | T | T | |
| 129 | N | — | NHQ | NQ | — | |
| 134 | L | MW | M | M | [symbol] G [symbol] | FMNR (SEQ |
| | | | | | | ID NO: 2121) |
| 135 | S | Q | RQ | Q | Q | |
| 136 | A | SAD | — | — | — | |
| 138 | G | — | [symbol] RA | — | — | |
| 139 | H | I | I | I | I | |
| 140 | F | Y | F [symbol] | Y | Y | |
| 144 | C | WAS [symbol] | S [symbol] | — | — | |
| | | (SEQ ID | | | | |
| | | NO: 2122) | | | | |
| 145 | V | VA | — | — | — | |
| 147 | E | — | [symbol] VW | L | L | |
| 151 | H | L | [symbol] RKM (SEQ | L | L | |
| | | | ID NO: 2123) | | | |
| 162 | T | — | QT | — | — | |
| 166 | M | MK | — | — | — | |
| 170 | L | VI | V | V | V | |
| 174 | I | L | LVW | L | FIL [symbol] (SEQ ID | [symbol] Y (SEQ ID |
| | | | | | NO: 2124) | NO: 2125) |
| 175 | E | EN | — | — | — | |
| 177 | F | K | [symbol] RQL (SEQ | K | H [symbol] R | [symbol] FNS (SEQ |
| | | | ID NO: 2126) | | | ID NO: 2127) |
| 183 | E | — | EDG | — | — | |
| 184 | P | PL | — | — | — | |
| 185 | A | — | AD | — | — | |
| 195 | C | SRAC (SEQ | SRAC (SEQ | S | S | |
| | | ID NO: 2128) | ID NO: 2128) | | | |
| 203 | C | SRAC (sEQ | SR [symbol] C (SEQ | A | A | |
| | | ID NO: 2128) | ID NO: 2128) | | | |

(—) = same as TetR
Italic = biased incorporation by design
BOLD and Oversized = Bias from screening
Residues in parentheses = unintended mutations

TABLE 6A
Oligonucleotides for assembly and rescue of Libraries L10 and
L11.

| Oligo | | | Pool |
|---|---|---|---|
| Name | SEQ ID No | Sequence | # |
| L10:1 | 890 | TGGCACGTCAAGAACAAGCGAGCTCTGCTAGACGCTATGGCC | 10a |
| L10:2 | 891 | ATCGAGATGCTCGATCSCCACGCTATACACTWCTTACYCTTG | 10b |
| L10:3 | 892 | TTCGAGATGCTCGATCSCCACGCTATACACTWCTTACYCTTG | |
| L10:4 | 893 | ATCGAGATGCTCGATCSCCACGCTATACACTWCWGTCYCTTG | |
| L10:5 | 894 | TTCGAGATGCTCGATCSCCACGCTATACACTWCWGTCYCTTG | |
| L10:6 | 895 | ATCGAGATGCTCGATCSCCACGCTATACACTTGTTACYCTTG | |
| L10:7 | 898 | TTCGAGATGCTCGATCSCCACGCTATACACTTGTTACYCTTG | |
| L10:8 | 897 | ATCGAGATGCTCGATCSCCACGCTATACACTTGWGTCYCTTG | |
| L10:9 | 898 | TTCGAGATGCTCGATCSCCACGCTATACACTTGWGTCYCTTG | |
| L10:10 | 899 | ATCGAGATGCTCGATCSCCACGCTMCCCACTWCTTACYCTTG | |
| L10:11 | 900 | TTCGAGATGCTCGATCSCCACGCTMCCCACTWCTTACYCTTG | |
| L10:12 | 901 | ATCGAGATGCTCGATCSCCACGCTMCCCACTWCWGTCYCTTG | |
| L10:13 | 902 | TTCGAGATGCTCGATCSCCACGCTMCCCACTWCWGTCYCTTG | |
| L10:14 | 903 | ATCGAGATGCTCGATCSCCACGCTMCCCACTTGTTACYCTTG | |
| L10:15 | 904 | TTCGAGATGCTCGATCSCCACGCTMCCCACTTGTTACYCTTG | |
| L10:16 | 905 | ATCGAGATGCTCGATCSCCACGCTMCCCACTTGWGTCYCTTG | |
| L10:17 | 906 | TTCGAGATGCTCGATCSCCACGCTMCCCACTTGWGTCYCTTG | |
| L10:18 | 907 | GAAGGGGMAAGCTGGCAAGACTTCTTGAGGAACAAMGCTAAG | 10c |
| L10:19 | 908 | TCCATGAGAAACGCTTTGCTCAGTCACCGTGATGGAGCCAAG | 10d |
| L10:20 | 909 | TCCATGAGAYGTGCTTTGCTCAGTCACCGTGATGGAGCCAAG | |
| L10:21 | 910 | GCGTGTCTAGGTACGGGCTTMACGGAGCAAAACTATGAAACT | 10e |
| L10:22 | 911 | GTGTGTCTAGGTACGGGCTTMACGGAGCAAAACTATGAAACT | |
| L10:23 | 912 | GCGTGTCTAGGTACGGGCTTMACGGAGCAACAATATGAAACT | |
| L10:24 | 913 | GTGTGTCTAGGTACGGGCTTMACGGAGCAACAATATGAAACT | |
| L10:25 | 914 | ACGGAGAACMGCCTTGCCTTCCTGTGTCAACAAGGTTTCTCC | 10f |
| L10:26 | 915 | GCGGAGAACMGCCTTGCCTTCCTGTGTCAACAAGGTTTCTCC | |
| L10:27 | 916 | ACGGAGAACMGCCTTGCCTTCCTGACGCAACAAGGTTTCTCC | |
| L10:28 | 917 | GCGGAGAACMGCCTTGCCTTCCTGACGCAACAAGGTTTCTCC | |
| L10:29 | 918 | CTTGAGAACGCCCTCTACGCATGGCAAGACSTGGGGATCTAC | 10g |
| L10:30 | 919 | CTTGAGAACGCCCTCTACGCATGGCAAKCASTGGGGATCTAC | |
| L10:31 | 920 | CTTGAGAACGCCCTCTACGCAATGCAAGACSTGGGGATCTAC | |
| L10:32 | 921 | CTTGAGAACGCCCTCTACGCAATGCAAKCASTGGGGATCTAC | |
| L10:33 | 922 | ACTCTGGGTTGSGYGTTGCTGGATCAAGAGCTGCAAGTCGCT | 10h |
| L10:34 | 923 | ACTCTGGGTKCGGYGTTGCTGGATCAAGAGCTGCAAGTCGCT | |
| L10:35 | 924 | AAGGAGGAGAGGGAAACACCTACTACTGATAGTAWGCCGCCA | 10i |
| L10:36 | 925 | CTGRTACGACAAGCTCTGAACCTCAAGGATCACCAAGGTGCA | 10j |
| L10:37 | 926 | CTGRTACGACAAGCTCTGGAACTCAAGGATCACCAAGGTGCA | |
| L10:38 | 927 | GAGCYCGCCTTCCTGTTCGGCCTTGAACTGATCATAGCTGGA | 10k |
| L10:39 | 928 | GAGCYCGCCTTCCTGTTCGGCCTTGAACTGATCATAHGCGGA | |
| L10:40 | 929 | TTGGAGAAGCAGCTGAAGGCTGAAAGTGGGTCTTAATGATAG | 10L |
| L10:41 | 930 | TTGGAGAAGCAGCTGAAGHGTGAAAGTGGGTCTTAATGATAG | |
| L10:42 | 931 | GTGGSGATCGAGCATCTCGAWGGCCATAGCGTCTAGCAGAGC | 10m |
| L10:43 | 932 | GTCTTGCCAGCTTKCCCCTTCCAAGRGTAAGWAGTGTATAGC | 10n |
| L10:44 | 933 | GTCTTGCCAGCTTKCCCCTTCCAAGRGACWGWAGTGTATAGC | |
| L10:45 | 934 | GTCTTGCCAGCTTKCCCCTTCCAAGRGTAACAAGTGTATAGC | |
| L10:46 | 935 | GTCTTGCCAGCTTKCCCCTTCCAAGRGACWCAAGTGTATAGC | |
| L10:47 | 935 | GTCTTGCCAGCTTKCCCCTTCCAAGRGTAAGWAGTGGGKAGC | |
| L10:48 | 937 | GTCTTGCCAGCTTKCCCCTTCCAAGRGACWGWAGTGGGKAGC | |
| L10:49 | 938 | GTCTTGCCAGCTTKCCCCTTCCAAGRGTAACAAGTGGGKAGC | |
| L10:50 | 939 | GTCTTGCCAGCTTKCCCCTTCCAAGRGACWCAAGTGGGKAGC | |
| L10:51 | 940 | GAGCAAAGCGTTTCTCATGGACTTAGCKTTGTTCCTCAAGAA | 10o |
| L10:52 | 941 | GAGCAAAGCACRTCTCATGGACTTAGCKTTGTTCCTCAAGAA | |
| L10:53 | 942 | GAAGCCCGTACCTAGACACRCCTTGGCTCCATCACGGTGACT | 10p |
| L10:54 | 943 | TAAGCCCGTACCTAGACACRCCTTGGCTCCATCACGGTGACT | |
| L10:55 | 944 | GAAGGCAAGGCKGTTCTCCGYAGTTTCATAGTTTTGCTCCGT | 10q |
| L10:56 | 945 | GAAGGCAAGGCKGTTCTCCGYAGTTTCATATTGTTGCTCCGT | |
| L10:57 | 946 | TGCGTAGAGGGCGTTCTCAAGGGAGAAACCTTGTTGACACAG | 10r |
| L10:58 | 947 | TGCGTAGAGGGCGTTCTCAAGGGAGAAACCTTGTTGCGTCAG | |
| L10:59 | 948 | CAGCAACRCSCAACCCAGAGTGTAGATCCCCASGTCTTGCCA | 10s |
| L10:60 | 949 | CAGCAACRCCGMACCCAGAGTGTAGATCCCCASGTCTTGCCA | |
| L10:61 | 950 | CAGCAACRCSCAACCCAGAGTGTAGATCCCCASTGMTTGCCA | |
| L10:62 | 951 | CAGCAACRCCGMACCCAGAGTGTAGATCCCCASTGMTTGCCA | |
| L10:63 | 952 | CAGCAACRCSCAACCCAGAGTGTAGATCCCCASGTCTTGCAT | |
| L10:64 | 953 | CAGCAACRCCGMACCCAGAGTGTAGATCCCCASGTCTTGCAT | |
| L10:65 | 954 | CAGCAACRCSCAACCCAGAGTGTAGATCCCCASTGMTTGCAT | |
| L10:66 | 955 | CAGCAACRCCGMACCCAGAGTGTAGATCCCCASTGMTTGCAT | |
| L10:67 | 956 | AGGTGTTTCCCTCTCCTCCTTAGCGACTTGCAGCTCTTGATC | 10t |
| L10:68 | 957 | GTTCAGAGCTTGTCGTAYCAGTGGCGGCWTACTATCAGTAGT | 10u |
| L10:69 | 968 | TTCCAGAGCTTGTCGTAYCAGTGGCGGCWTACTATCAGTAGT | |
| L10:70 | 959 | GCCGAACAGGAAGGCGRGCTCTGCACCTTGGTGATCCTTGAG | 10v |
| L10:71 | 960 | AGCCTTCAGCTGCTTCTCCAATCCAGCTATGATCAGTTCAAG | 10w |
| L10:72 | 961 | ACGCTTCAGCTGCTTCTCCAATCCAGCTATGATCAGTTCAAG | |
| L10:73 | 962 | ACTCTTCAGCTGCTTCTCCAATCCAGCTATGATCAGTTCAAG | |
| L10:74 | 963 | ACACTTCAGCTGCTTCTCCAATCCAGCTATGATCAGTTCAAG | |
| L10:75 | 964 | AGCCTTCAGCTGCTTCTCCAATCCGCDTATGATCAGTTCAAG | |
| L10:76 | 966 | ACGCTTCAGCTGCTTCTCCAATCCGCDTATGATCAGTTCAAG | |
| L10:77 | 966 | ACTCTTCAGCTGCTTCTCCAATCCGCDTATGATCAGTTCAAG | |
| L10:78 | 967 | ACACTTCAGCTGCTTCTCCAATCCGCDTATGATCAGTTCAAG | |
| L10:79 | 988 | GCGCCAAGGTACCTTCTGCAGCTATCATTAAGACCCACTTTC | 10x |

TABLE 6B

| | SEQ | | |
|---|---|---|---|
| Oligo | ID | | Pool |
| Name | NO | Sequence | # |
| L11:1 | 969 | TGGCACGTCAAGAACAAGCGAGCTCTGCTAGA | 11a |
| | | CGCTATGGCC | |
| L11:2 | 970 | ATTGAGATGCTCAACAGGCACGCTACCCASTA | 11b |
| | | CCTACCTTTG | |
| L11:3 | 971 | ATTGAGATGCTCAACAGGCACGCTACCCASTA | |
| | | CTSTCCTTTG | |
| L11:4 | 972 | ATTGAGATGCTCAACAGGCACGCTACCTATTA | |
| | | CCTACCTTTG | |
| L11:5 | 973 | ATTGAGATGCTCAACAGGCACGCTACCTATTA | |
| | | CTSTCCTTTG | |
| L11:6 | 974 | ATTGAGATGCTCGAKAGGCACGCTACCCASTA | |
| | | CCTACCTTTG | |
| L11:7 | 975 | ATTGAGATGCTCGAKAGGCACGCTACCCASTA | |
| | | CTSTCCTTTG | |
| L11:8 | 976 | ATTGAGATGCTCGAKAGGCACGCTACCTATTA | |
| | | CCTACCTTTG | |
| L11:9 | 977 | ATTGAGATGCTCGAKAGGCACGCTACCTATTA | |
| | | CTSTCCTTTG | |
| L11:10 | 978 | GWGGGGGAAAGCTGGCAARATTTCTTGAGGAA | 11c |
| | | CAACGCTAAG | |
| L11:11 | 979 | TCCATGAGAAATGCTTTGCTCAGTCACCGTGA | 11d |
| | | TGGAGCCAAG | |
| L11:12 | 980 | TCCATGAGAYGTGCTTTGCTCAGTCACCGTGA | |
| | | TGGAGCCAAG | |
| L11:13 | 981 | GTCTGTCTAGGTACGGSGDTCACGGAGAACCA | 11e |
| | | GTATGAAACT | |
| L11:14 | 982 | GTCTGTCTAGGTACGGSGDTCACGGAGCAACA | |
| | | GTATGAAACT | |
| L11:15 | 983 | GTCTGTCTAGGTACGGSGTGGACGGAGAACCA | |
| | | GTATGAAACT | |
| L11:16 | 984 | GTCTGTCTAGGTACGGSGTGGACGGAGCAACA | |
| | | GTATGAAACT | |
| L11:17 | 985 | CTTGAGAACTCACTTGCCTTCCTGTGCCAACA | 11f |
| | | AGGTTTCTCC | |
| L11:18 | 986 | GTTGAGAACTCACTTGCCTTCCTGTGCCAACA | |
| | | AGGTTTCTCC | |
| L11:19 | 987 | ATTGAGAACTCACTTGCCTTCCTGTGCCAACA | |
| | | AGGTTTCTCC | |
| L11:20 | 988 | CTTGAGAACTCACTTGCCTTCCTGACGCAACA | |
| | | AGGTTTCTCC | |
| L11:21 | 989 | GTTGAGAACTCACTTGCCTTCCTGACGCAACA | |
| | | AGGTTTCTCC | |
| L11:22 | 990 | ATTGAGAACTCACTTGCCTTCCTGACGCAACA | |
| | | AGGTTTCTCC | |
| L11:23 | 991 | CTTGAGAACCAGCTTGCCTTCCTGTGCCAACA | |
| | | AGGTTTCTCC | |
| L11:24 | 992 | GTTGAGAACCAGCTTGCCTTCCTGTGCCAACA | |
| | | AGGTTTCTCC | |
| L11:25 | 993 | ATTGAGAACCAGCTTGCCTTCCTGTGCCAACA | |
| | | AGGTTTCTCC | |
| L11:26 | 994 | CTTGAGAACCAGCTTGCCTTCCTGACGCAACA | |
| | | AGGTTTCTCC | |
| L11:27 | 995 | GTTGAGAACCAGCTTGCCTTCCTGACGCAACA | |
| | | AGGTTTCTCC | |
| L11:28 | 996 | ATTGAGAACCAGCTTGCCTTCCTGACGCAACA | |
| | | AGGTTTCTCC | |
| L11:29 | 997 | CTTGAGAACATGCTTGCCTTCCTGTGCCAACA | |
| | | AGGTTTCTCC | |
| L11:30 | 998 | GTTGAGAACATGCTTGCCTTCCTGTGCCAACA | |
| | | AGGTTTCTCC | |
| L11:31 | 999 | ATTGAGAACATGCTTGCCTTCCTGTGCCAACA | |
| | | AGGTTTCTCC | |
| L11:32 | 1000 | CTTGAGAACATGCTTGCCTTCCTGACGCAACA | |
| | | AGGTTTCTCC | |
| L11:33 | 1001 | GTTGAGAACATGCTTGCCTTCCTGACGCAACA | |
| | | AGGTTTCTCC | |
| L11:34 | 1002 | ATTGAGAACATGCTTGCCTTCCTGACGCAACA | |
| | | AGGTTTCTCC | |
| L11:35 | 1003 | GCCGAGAACTCACTTGCCTTCCTGTGCCAACA | |
| | | AGGTTTCTCC | |
| L11:36 | 1004 | GCCGAGAACTCACTTGCCTTCCTGACGCAACA | |
| | | AGGTTTCTCC | |
| L11:37 | 1005 | GCCGAGAACCAGCTTGCCTTCCTGTGCCAACA | |
| | | AGGTTTCTCC | |
| L11:38 | 1006 | GCCGAGAACCAGCTTGCCTTCCTGACGCAACA | |
| | | AGGTTTCTCC | |
| L11:39 | 1007 | GCCGAGAACATGCTTGCCTTCCTGTGCCAACA | |
| | | AGGTTTCTCC | |
| L11:40 | 1008 | GCCGAGAACATGCTTGCCTTCCTGACGCAACA | |
| | | AGGTTTCTCC | |
| L11:41 | 1009 | CTTGAGAATGCCCTCTACGCAATGCRGGCTGT | 11g |
| | | TCGGATCTWC | |
| L11:42 | 1010 | CTTGAGAATGCCCTCTACGCAATGCRGGCTGT | |
| | | TGSCATCTWC | |
| L11:43 | 1011 | CTTGAGCAWGCCCTCTACGCAATGCRGGCTGT | |
| | | TCGGATCTWC | |
| L11:44 | 1012 | CTTGAGCAWGCCCTCTACGCAATGCRGGCTGT | |
| | | TGSCATCTWC | |
| L11:45 | 1013 | ACTCTGGGTTSCGTCTTGTGGGATCAAGAGCT | 11h |
| | | ACAAGTCGCT | |
| L11:46 | 1014 | ACTCTGGGTTSCGTCTTGTGGGATCAAGAGAD | |
| | | GCAAGTCGCT | |
| L11:47 | 1015 | ACTCTGGGTTSCGTCTTGSTAGATCAAGAGCT | |
| | | ACAAGTCGCT | |
| L11:48 | 1016 | ACTCTGGGTTSCGTCTTGSTAGATCAAGAGAD | |
| | | GCAAGTCGCT | |
| L11:49 | 1017 | AAGGAGGAGAGGGAAACACCTACTACTGATAG | 11i |
| | | TATGCCGCCA | |
| L11:50 | 1018 | AAGGAGGAGAGGGAAACACCTCAGACTGATAG | |
| | | TATGCCGCCA | |
| L11:51 | 1019 | CTGGTTCGACAAGCTKTGGAACTCCDGGATCA | 11j |
| | | CCAAGGTGCA | |
| L11:52 | 1020 | CTGGTTCGACAAGCTKTGGAACTCAAAGATCA | |
| | | CCAAGGTGCA | |
| L11:53 | 1021 | CTGGTTCGACAAGCTTGGGAACTCCDGGATCA | |
| | | CCAAGGTGCA | |
| L11:54 | 1022 | CTGGTTCGACAAGCTTGGGAACTCAAAGATCA | |
| | | CCAAGGTGCA | |
| L11:55 | 1023 | GRWCCAGMTTTCCTGTTCGGCCTTGAACTGAT | 11k |
| | | CATAGCAGGA | |
| L11:56 | 1024 | GRWCCAGMTTTCCTGTTCGGCCTTGAACTGAT | |
| | | CATAHGCGGA | |
| L11:57 | 1025 | TTGGAGAAGCAGCTGAAGHGCGAAAGTGGGTC | 11L |
| | | TTAATGATAG | |
| L11:58 | 1026 | TTGGAGAAGCAGCTGAAGGCGGAAAGTGGGTC | |
| | | TTAATGATAG | |
| L11:59 | 1027 | GTGCCTGTTGAGCATCTCAATGGCCATAGCGT | 11m |
| | | CTAGCAGAGC | |
| L11:60 | 1028 | GTGCCTMTCGAGCATCTCAATGGCCATAGCGT | |
| | | CTAGCAGAGC | |
| L11:61 | 1029 | ATYTTGCCAGCTTTCCCCCWCCAAAGGTAGGT | 11n |
| | | ASTGGGTAGC | |
| L11:62 | 1030 | ATYTTGCCAGCTTTCCCCCWCCAAAGGASAGT | |
| | | ASTGGGTAGC | |
| L11:63 | 1031 | ATYTTGCCAGCTTTCCCCCWCCAAAGGTAGGT | |
| | | AATAGGTAGC | |
| L11:64 | 1032 | ATYTTGCCAGCTTTCCCCCWCCAAAGGASAGT | |
| | | AATAGGTAGC | |
| L11:65 | 1033 | GAGCAAAGCATTTCTCATGGACTTAGCGTTGT | 11o |
| | | TCCTCAAGAA | |
| L11:66 | 1034 | GAGCAAAGCACRTCTCATGGACTTAGCGTTGT | |
| | | TCCTCAAGAA | |
| L11:67 | 1035 | GAHCSCCGTACCTAGACAGACCTTGGCTCCAT | 11p |
| | | CACGGTGACT | |
| L11:68 | 1036 | CCACSCCGTACCTAGACAGACCTTGGCTCCAT | |
| | | CACGGTGACT | |
| L11:69 | 1037 | GAAGGCAAGTGAGTTCTCAABAGTTTCATACT | 11q |
| | | GGTTCTCCGT | |
| L11:70 | 1038 | GAAGGCAAGCTGGTTCTCAABAGTTTCATACT | |
| | | GGTTCTCCGT | |
| L11:71 | 1039 | GAAGGCAAGCATGTTCTCAABAGTTTCATACT | |
| | | GGTTCTCCGT | |
| L11:72 | 1040 | GAAGGCAAGTGAGTTCTCGGCAGTTTCATACT | |
| | | GGTTCTCCGT | |
| L11:73 | 1041 | GAAGGCAAGCTGGTTCTCGGCAGTTTCATACT | |
| | | GGTTCTCCGT | |
| L11:74 | 1042 | GAAGGCAAGCATGTTCTCGGCAGTTTCATACT | |
| | | GGTTCTCCGT | |
| L11:75 | 1043 | GAAGGCAAGTGAGTTCTCAABAGTTTCATACT | |
| | | GTTGCTCCGT | |
| L11:76 | 1044 | GAAGGCAAGCTGGTTCTCAABAGTTTCATACT | |
| | | GTTGCTCCGT | |
| L11:77 | 1045 | GAAGGCAAGCATGTTCTCAABAGTTTCATACT | |
| | | GTTGCTCCGT | |
| L11:78 | 1046 | GAAGGCAAGTGAGTTCTCGGCAGTTTCATACT | |
| | | GTTGCTCCGT | |
| L11:79 | 1047 | GAAGGCAAGCTGGTTCTCGGCAGTTTCATACT | |
| | | GTTGCTCCGT | |
| L11:80 | 1048 | GAAGGCAAGCATGTTCTCGGCAGTTTCATACT | |
| | | GTTGCTCCGT | |
| L11:81 | 1049 | TGCGTAGAGGGCATTCTCAAGGGAGAAACCTT | 11r |
| | | GTTGGCACAG | |
| L11:82 | 1050 | TGCGTAGAGGGCWTGCTCAAGGGAGAAACCTT | |
| | | GTTGGCACAG | |
| L11:83 | 1051 | TGCGTAGAGGGCATTCTCAAGGGAGAAACCTT | |
| | | GTTGCGTCAG | |
| L11:84 | 1052 | TGCGTAGAGGGCWTGCTCAAGGGAGAAACCTT | |
| | | GTTGCGTCAG | |
| L11:85 | 1053 | CCACAAGACGSAACCCAGAGTGWAGATCCGAA | 11s |
| | | CAGCCYGCAT | |
| L11:86 | 1054 | TASCAAGACGSAACCCAGAGTGWAGATCCGAA | |
| | | CAGCCYGCAT | |
| L11:87 | 1055 | CCACAAGACGSAACCCAGAGTGWAGATGSCAA | |
| | | CAGCCYGCAT | |
| L11:88 | 1056 | TASCAAGACGSAACCCAGAGTGWAGATGSCAA | |
| | | CAGCCYGCAT | |
| L11:89 | 1057 | AGGTGTTTCCCTCTCCTCCTTAGCGACTTGTA | 11t |
| | | GCTCTTGATC | |
| L11:90 | 1058 | AGGTGTTTCCCTCTCCTCCTTAGCGACTTGCH | |
| | | TCTCTTGATC | |
| L11:91 | 1059 | TTCCAMAGCTTGTCGAACCAGTGGCGGCATAC | 11u |
| | | TATCAGTAGT | |
| L11:92 | 1060 | TTCCCAAGCTTGTCGAACCAGTGGCGGCATAC | |
| | | TATCAGTAGT | |
| L11:93 | 1061 | TTCCAMAGCTTGTCGAACCAGTGGCGGCATAC | |
| | | TATCAGTCTG | |
| L11:94 | 1062 | TTCCCAAGCTTGTCGAACCAGTGGCGGCATAC | |
| | | TATCAGTCTG | |
| L11:95 | 1063 | GCCGAACAGGAAAKCTGGWYCTGCACCTTGGT | 11v |
| | | GATCCHGGAG | |
| L11:96 | 1064 | GCCGAACAGGAAAKCTGGWYCTGCACCTTGGT | |
| | | GATCTTTGAG | |
| L11:97 | 1065 | GCDCTTCAGCTGCTTCTCCAATCCTGCTATGA | 11w |
| | | TCAGTTCAAG | |
| L11:98 | 1066 | CGCCTTCAGCTGCTTCTCCAATCCTGCTATGA | |
| | | TCAGTTCAAG | |
| L11:99 | 1067 | GCDCTTCAGCTGCTTCTCCAATCCGCDTATGA | |
| | | TCAGTTCAAG | |
| L11:100 | 1068 | CGCCTTCAGCTGCTTCTCCAATCCGCDTATGA | |
| | | TCAGTTCAAG | |
| L11:101 | 1069 | GCGCCAAGGTACCTTCTGCAGCTATCATTAAG | 11x |
| | | ACCCACTTTC | |

TABLE 6C

| Oligo | SEQ ID | | |
|---|---|---|---|
| Name | NO | Sequence | Pool |
| EsRA11:1 | 1070 | TGGCACGTCAAGAACAAGCGAGCTCTGCTAGACGCTATGGCC | A11a |
| EsRA11:2 | 1071 | ATTGAGATGCTCGATAGGCACGCTACCCACTACTSTCCTTTG | A11b |
| EsRA11:3 | 1072 | ATTGAGATGCTCGATAGGCACGCTACCCACTACCTACCTTTG | |
| EsRA11:4 | 1073 | GAAGGGGAAAGCTGGCAAGACTTCTTGAGGAACAACGCTAAG | A11c |
| EsRA11:5 | 1074 | TCCATGAGAYGCGCTTTGCTCAGTCACCGTGATGGAGCCAAG | A11d |
| EsRA11:6 | 1075 | TCCATGAGAAATGCTTTGCTCAGTCACCGTGATGGAGCCAAG | |
| EsRA11:7 | 1076 | GTCTGTCTAGGTACGGGCTTCACGGAGCAACAGTATGAAACT | A11e |
| EsRA11:8 | 1077 | GCTGAGAACAGCCTTGCCTTCCTGACACAACAAGGTTTCTCC | A11f |
| EsRA11:9 | 1078 | GCTGAGAACAGCCTTGCCTTCCTGTGTCAACAAGGTTTCTCC | |
| EsRA11:10 | 1079 | CTTGAGAACGCCCICTACGCAATGCAAGCTGTTGGGATCTAC | A11g |
| EsRA11:11 | 1080 | ACTCTGGGTWGTGTCTTGCTGGATCAAGAGCTGCAAGTCGCT | A11h |
| EsRA11:12 | 1081 | AAGGAGGAGAGGGAAACACCTACTACTGATAGTATGCCGCCA | A11i |
| EsRA11:13 | 1082 | CTGGTTCGACAAGCTCTGGAACTCAAGGATCACCAAGGTGCA | A11j |
| EsRA11:14 | 1083 | GAGCCAGCCTTCCTGTTCGGCCTTGAACTGATCATAGCAGGA | A11k |
| EsRA11:15 | 1084 | GAGCCAGCCTTCCTGTTCGGCCTTGAACTGATCATAHGCGGA | |
| EsRA11:16 | 1085 | TTGGAGAAGCAGCTGAAGGCCGAAAGTGGGTCTTAATGATAG | A11L |
| EsRA11:17 | 1086 | TTGGAGAAGCAGCTGAAGHGTGAAAGTGGGTCTTAATGATAG | |
| EsRA11:18 | 1087 | GTGCCTATCGAGCATCTCAATGGCCATAGCGTCTAGCAGAGC | A11m |
| EsRA11:19 | 1088 | GTCTTGCCAGCTTTCCCCTTCCAAAGGASAGTAGTGGGTAGC | A11n |
| EsRA11:20 | 1089 | GTCTTGCCAGCTTTCCCCTTCCAAAGGTAGGTAGTGGGTAGC | |
| EsRA11:21 | 1090 | GAGCAAAGCGCRTCTCATGGACTTAGCGTTGTTCCTCAAGAA | A11o |
| EsRA11:22 | 1091 | GAGCAAAGCATTTCTCATGGACTTAGCGTTGTTCCTCAAGAA | |
| EsRA11:23 | 1092 | GAAGCCCGTACCTAGACAGACCTTGGCTCCATCACGGTGACT | A11p |
| EsRA11:24 | 1093 | GAAGGCAAGGCTGTTCTCAGCAGTTTCATACTGTTGCTCCGT | A11q |
| EsRA11:25 | 1094 | TGCGTAGAGGGCGTTCTCAAGGGAGAAACCTTGTTGTGTCAG | A11r |
| EsRA11:26 | 1095 | TGCGTAGAGGGCGTTCTCAAGGGAGAAACCTTGTTGACACAG | |
| EsRA11:27 | 1096 | CAGCAAGACACWACCCAGAGTGTAGATCCCAACAGCTTGCAT | A11s |
| EsRA11:28 | 1097 | AGGTGTTTCCCTCTCCTCCTTAGCGACTTGCAGCTCTTGATC | A11t |
| EsRA11:29 | 1098 | TTCCAGAGCTTGTCGAACCAGTGGCGGCATACTATCAGTAGT | A11u |
| EsRA11:30 | 1099 | GCCGAACAGGAAGGCTGGCTCTGCACCTTGGTGATCCTTGAG | A11v |
| EsRA11:31 | 1100 | GGCCTTCAGCTGCTTCTCCAATCCTGCTATGATCAGTTCAAG | A11w |
| EsRA11:32 | 1101 | ACDCTTCAGCTGCTTCTCCAATCCTGCTATGATCAGTTCAAG | |
| EsRA11:33 | 1102 | GGCCTTCAGCTGCTTCTCCAATCCGCDTATGATCAGTTCAAG | |
| EsRA11:34 | 1103 | ACDCTTCAGCTGCTTCTCCAATCCGCDTATGATCAGTTCAAG | |
| EsRA11:35 | 1104 | GCGCCAAGGTACCTTCTGCAGCTATCATTAAGACCCACTTTC | A11x |
| SynSU5′ | 1105 | CACGTCAAGAACAAGCGAGCTCTGCTAGAC | |
| SynSU3′ | 1106 | GGAACTTCGGCGCGCCAAGGTACCTTCTGCAGCTATC | |

Following library assembly and cloning approximately 100-L10 and 130-L11 putative hits were identified from ˜20,000 repressor positive clones. The clones were re-arrayed and ranked for repressor and ligand activity by relative colony color on M9 X-gal indicator (U.S. Utility application Ser. No. 13/086,765, filed on Apr. 14, 2011 and in US Application Publication 2010-0105141, both of which are herein incorporated by reference in their entirety) plates containing 0, 1.5 and 7 ppb ethametsulfuron. All putative hits and 180 random clones from each library were sequenced and the data sets compared to create sequence activity relationships (Table 5). Library 10 results show P69L, E73A, and N82K substitutions are biased in improved clones while C144 was strongly selected over the diversity as 31 vs. 11; 31 vs. 10; 28 vs. 4; and 85 vs. 42% of the hits contained these residues compared to the randomly selected population, respectively. Although 157F was poorly incorporated in the library (none in the random population), it was found in 5% of the hit population—mostly associated with the top ligand responsive clones. Incorporation data for L11 shows that residues G104, F105, Q108, A113, Q135, G138, Y140, C144, L147, L151, and K177 were all nearly 100% conserved. The results for positions 104, 105, 135, 147, and 151 corroborate the results for the in vitro mutagenesis study showing these residues to be highly important for activity. Additionally, residues 68C and S116 were also selectively maintained over optional diversity while C121T and C203A were both preferred as 71 vs. 45 and 56 vs. 35% of the respective hits vs. random clones contained these latter changes. Top hits from libraries L10 and L11 are shown in Table 7.

### B. Fifth Round Shuffling

One of the key and often overlooked aspects of any gene switch is maintenance of a very low level of expression in the ‘off’ state. To enhance the stringency of the in vivo repressor assay a new library vector, pVER7571, was constructed with a mutated ribosome binding site to lower the basal level of repressor produced in our assay strain and thus enhance the sensitivity of ‘leakiness’ detection. Library L12 was constructed in this new vector. Library L12 focused on reiterative shuffling of positive residue diversity from libraries L10 & L11 and (Table 5). Library L12 was constructed from thirty-two oligonucleotides (Table 8).

TABLE 8
Oligonucleotides for assembly of library L12.

| | | SEQ |
|---|---|---|
| | | ID |
| Oligo | Sequence | NO |
| L12:1 | TGGCACGTCAAGAACAAGCGAGCTCTGCTAGACGCTAT | 1107 |
| | GGCC | |
| L12:2 | ATCGAGATGCTCGATCSCCACGCTATACACTWTTTACY | 1108 |
| | ATTG | |
| L12:3 | TTCGAGATGCTCGATCSCCACGCTATACACTWTTTACY | 1109 |
| | ATTG | |
| L12:4 | ATCGAGATGCTCGATCSCCACGCTMCCCACTWTTTACY | 1110 |
| | ATTG | |
| L12:5 | TTCGAGATGCTCGATCSCCACGCTMCCCACTWTTTACY | 1111 |
| | ATTG | |
| L12:6 | GAAGGGGMAAGCTGGCAAAATTTCTTGAGGAACAAMGC | 1112 |
| | TAAG | |
| L12:7 | TCCATGAGAAACGCTTTGCTCAGTCACCGTGATGGAGC | 1113 |
| | CAAG | |
| L12:8 | GTCTGTCTAGGTACGGGCTTCACGGAGCAACAATATGA | 1114 |
| | AACT | |
| L12:9 | GCGGAGAACCGCCTTGCCTTCCTGACACAACAAGGTTT | 1115 |
| | CTCC | |
| L12:10 | CTTGAGAACGCCCTCTACGCATGGCAAGCAGTGGGGAT | 1116 |
| | CTAC | |
| L12:11 | CTTGAGCAGGCCCTCTACGCATGGCAAGCAGTGGGGAT | 1117 |
| | CTAC | |
| L12:12 | ACTCTGGGTTGTGTCTTGCTGGATCAAGAGCTGCAAGT | 1118 |
| | CGCT | |
| L12:13 | AAGGAGGAGAGGGAAACACCTACTACTGATAGTATGCC | 1119 |
| | GCCA | |
| L12:14 | CTGGTTCGACAAGCTKTAGAACTCAAGGATCACCAAGG | 1120 |
| | TGCA | |
| L12:15 | CTGGTTCGACAAGCTTGGGAACTCAAGGATCACCAAGG | 1121 |
| | TGCA | |
| L12:16 | GAGCCAGCCTTCCTGTTCGGCCTTGAACTGATCATATC | 1122 |
| | AGGA | |
| L12:17 | TTGGAGAAGCAGCTGAAGGCAGAAAGTGGGTCTTAATG | 1123 |
| | ATAG | |
| L12:18 | GTGGSGATCGAGCATCTCGAWGGCCATAGCGTCTAGCA | 1124 |
| | GAGC | |
| L12:19 | ATTTTGCCAGCTTKCCCCTTCCAATRGTAAAWAGTGTA | 1125 |
| | TAGC | |
| L12:20 | ATTTTGCCAGCTTKCCCCTTCCAATRGTAAAWAGTGGG | 1126 |
| | KAGC | |
| L12:21 | GAGCAAAGCGTTTCTCATGGACTTAGCKTTGTTCCTCA | 1127 |
| | AGAA | |
| L12:22 | GAAGCCCGTACCTAGACAGACCTTGGCTCCATCACGGT | 1128 |
| | GACT | |
| L12:23 | GAAGGCAAGGCGGTTCTCCGCAGTTTCATATTGTTGCT | 1129 |
| | CCGT | |
| L12:24 | TGCGTAGAGGGCGTTCTCAAGGGAGAAACCTTGTTGTG | 1130 |
| | TCAG | |
| L12:25 | TGCGTAGAGGGCCTGCTCAAGGGAGAAACCTTGTTGTG | 1131 |
| | TCAG | |
| L12:26 | CAGCAAGACACAACCCAGAGTGTAGATCCCCACTGCTT | 1132 |
| | GCCA | |
| L12:27 | AGGTGTTTCCCTCTCCTCCTTAGCGACTTGCAGCTCTT | 1133 |
| | GATC | |
| L12:28 | TTCTAMAGCTTGTCGAACCAGTGGCGGCATACTATCAG | 1134 |
| | TAGT | |
| L12:29 | TTCCCAAGCTTGTCGAACCAGTGGCGGCATACTATCAG | 1135 |
| | TAGT | |
| L12:30 | GCCGAACAGGAAGGCTGGCTCTGCACCTTGGTGATCCT | 1136 |
| | TGAG | |
| L12:31 | TGCCTTCAGCTGCTTCTCCAATCCTGATATGATCAGTT | 1137 |
| | CAAG | |
| L12:32 | GCGCCAAGGTACCTTCTGCAGCTATCATTAAGACCCAC | 1138 |
| | TTTC | |

Approximately 10,000 clones from library L12 were screened using the genetic plate assay with no inducer to detect leaky B-gal expression and then addition of 2 ppb ethametsulfuron plus and minus 0.002% arabinose. The latter treatment increases the stringency of induction since arabinose induces repressor production. Sixty-six putative hits were ranked for activity and their sequences determined. Sequences were also determined from a population of 94 random clones and the two data sets compared. The data showed that wt TetR residues 157, R62, P69, E73, and N82 and substitutions T651 and F67Y were preferred. With the exception of E73 and N82 the preferences were modest. An alignment of the top hits from L12 is shown in Table 7.

### C. Sixth Round Shuffling

A sixth round of shuffling using vector pVER7571 incorporated the best diversity from Rd5 shuffling (Table 5). The fully synthetic library was constructed from oligonucleotides shown in Table 9. 7,500 clones were screened by the M9 X-gal plate based assay for repression in the absence of any inducers and induction in the presence of 2 ppb Es+/−0.002% arabinose. Forty-six putative hits were re-arrayed and replica plated onto the same series of M9 X-gal assay plates. The hits were ranked for induction and repression and their sequences determined in addition to 92 randomly selected clones. Sequence analysis of the hit population show that N82, W116, and to a lesser extent Y174 were strongly selected against relative to the alternative diversity (2 vs 25; 0 vs. 41; and 9 vs. 45%, respectively). Also, within the top performing group of hits W82, F134, A177, and to a lesser degree Q108 were selected for improved activity relative to the alternative diversity at these positions. Sequences of L15 hits are shown in Table 7.

TABLE 9
Oligonucleotides for assembly of library L15.

| | | SEQ |
|---|---|---|
| Oligo | | ID |
| Name | Sequence | NO |
| L15:1 | TGGCACGTCAAGAACAAGCGAGCTCTGCTAGACGCTA | 1139 |
| | TGGCC | |
| L15:2 | ATAGAGATGCTCGATCSGCACCAAAYTCACTACTTAC | 1140 |
| | CCTTG | |
| L15:3 | ATAGAGATGCTCGATCSGCACAVGAYTCACTACTTAC | 1141 |
| | CCTTG | |
| L15:4 | GAAGGGGAAAGCTGGCAARATTTCTTGAGGAACWGGG | 1142 |
| | CTAAG | |
| L15:5 | GAAGGGGAAAGCTGGCAARATTTCTTGAGGAACAAKG | 1143 |
| | CTAAG | |
| L15:6 | TCCATGAGAAATGCTTTGCTCAGTCACCGTGATGGAG | 1144 |
| | CCAAG | |
| L15:7 | GTCGCACTAGGTACGGGCTTCACGGAGMRACAATATG | 1145 |
| | AAACT | |
| L15:8 | GTCTGTCTAGGTACGGGCTTCACGGAGMRACAATATG | 1146 |
| | AAACT | |
| L15:9 | ATGGAGAACTSGCTTGCCTTCCTGACACAACAAGGTT | 1147 |
| | TCTCC | |
| L15:10 | ATGGAGAACAASCTTGCCTTCCTGACACAACAAGGTT | 1148 |
| | TCTCC | |
| L15:11 | CAAGAGAACTSGCTTGCCTTCCTGACACAACAAGGTT | 1149 |
| | TCTCC | |
| L15:12 | CAAGAGAACAASCTTGCCTTCCTGACACAACAAGGTT | 1150 |
| | TCTCC | |
| L15:13 | GCTGAGAACTSGCTTGCCTTCCTGACACAACAAGGTT | 1151 |
| | TCTCC | |
| L15:14 | TCTGAGAACTSGCTTGCCTTCCTGACACAACAAGGTT | 1152 |
| | TCTCC | |
| L15:15 | GCTGAGAACAASCTTGCCTTCCTGACACAACAAGGTT | 1153 |
| | TCTCC | |
| L15:16 | TCTGAGAACAASCTTGCCTTCCTGACACAACAAGGTT | 1154 |
| | TCTCC | |
| L15:17 | CTTGAGAACGCCCTCTACGCATTCCAAGCAGTGGGGA | 1155 |
| | TCTAC | |
| L15:18 | CTTGAGAACGCCCTCTACGCAAKGCAAGCAGTGGGGA | 1156 |
| | TCTAC | |
| L15:19 | CTTGAGAACGCCCTCTACGCAAATCAAGCAGTGGGGA | 1157 |
| | TCTAC | |
| L15:20 | ACTCTGGGTTGTGTCTTGCTGGATCAAGAGCTGCAAG | 1158 |
| | TCGCT | |
| L15:21 | AAGGAGGAGAGGGAAACACCTACTACTGATAGTATGC | 1159 |
| | CGCCA | |
| L15:22 | CTGGTTCGACAAGCTTACGAACTCGCGGATCACCAAG | 1160 |
| | GTGCA | |
| L15:23 | CTGGTTCGACAAGCTTACGAACTCTYCGATCACCAAG | 1161 |
| | GTGCA | |
| L15:24 | CTGGTTCGACAAGCTTACGAACTCAATGATCACCAAG | 1162 |
| | GTGCA | |
| L15:25 | CTGGTTCGACAAGCTDTTGAACTCGCGGATCACCAAG | 1163 |
| | GTGCA | |
| L15:26 | CTGGTTCGACAAGCTDTTGAACTCTYCGATCACCAAG | 1164 |
| | GTGCA | |
| L15:27 | CTGGTTCGACAAGCTDTTGAACTCAATGATCACCAAG | 1165 |
| | GTGCA | |
| L15:28 | GAGCCAGCCTTCCTGTTCGGCCTTGAACTGATCATAT | 1166 |
| | CAGGA | |
| L15:29 | TTGGAGAAGCAGCTGAAGGCCGAAAGTGGGTCTTAAT | 1167 |
| | GATAG | |
| L15:30 | GTGCSGATCGAGCATCTCTATGGCCATAGCGTCTAGC | 1168 |
| | AGAGC | |
| L15:31 | ATYTTGCCAGCTTTCCCCTTCCAAGGGTAAGTAGTGA | 1169 |
| | RTTTG | |
| L15:32 | ATYTTGCCAGCTTTCCCCTTCCAAGGGTAAGTAGTGA | 1170 |
| | RTCBT | |
| L15:33 | GAGCAAAGCATTTCTCATGGACTTAGCCCWGTTCCTC | 1171 |
| | AAGAA | |
| L15:34 | GAGCAAAGCATTTCTCATGGACTTAGCMTTGTTCCTC | 1172 |
| | AAGAA | |
| L15:35 | GAAGCCCGTACCTAGTGCGACCTTGGCTCCATCACGG | 1173 |
| | TGACT | |
| L15:36 | GAAGCCCGTACCTAGACAGACCTTGGCTCCATCACGG | 1174 |
| | TGACT | |
| L15:37 | GAAGGCAAGCSAGTTCTCCATAGTTTCATATTGTYKC | 1175 |
| | TCCGT | |
| L15:38 | GAAGGCAAGSTTGTTCTCCATAGTTTCATATTGTYKC | 1176 |
| | TCCGT | |
| L15:39 | GAAGGCAAGCSAGTTCTCTTGAGTTTCATATTGTYKC | 1177 |
| | TCCGT | |
| L15:40 | GAAGGCAAGSTTGTTCTCTTGAGTTTCATATTGTYKC | 1178 |
| | TCCGT | |
| L15:41 | GAAGGCAAGCSAGTTCTCAGMAGTTTCATATTGTYKC | 1179 |
| | TCCGT | |
| L15:42 | GAAGGCAAGSTTGTTCTCAGMAGTTTCATATTGTYKC | 1180 |
| | TCCGT | |
| L15:43 | TGCGTAGAGGGCGTTCTCAAGGGAGAAACCTTGTTGT | 1181 |
| | GTCAG | |
| L15:44 | CAGCAAGACACAACCCAGAGTGTAGATCCCCACTGCT | 1182 |
| | TGGAA | |
| L15:45 | CAGCAAGACACAACCCAGAGTGTAGATCCCCACTGCT | 1183 |
| | TGCMT | |
| L15:46 | CAGCAAGACACAACCCAGAGTGTAGATCCCCACTGCT | 1184 |
| | TGATT | |
| L15:47 | AGGTGTTTCCCTCTCCTCCTTAGCGACTTGCAGCTCT | 1185 |
| | TGATC | |
| L15:48 | TTCGTAAGCTTGTCGAACCAGTGGCGGCATACTATCA | 1186 |
| | GTAGT | |
| L15:49 | TTCAAHAGCTTGTCGAACCAGTGGCGGCATACTATCA | 1187 |
| | GTAGT | |
| L15:50 | GCCGAACAGGAAGGCTGGCTCTGCACCTTGGTGATCC | 1188 |
| | GCGAG | |
| L15:51 | GCCGAACAGGAAGGCTGGCTCTGCACCTTGGTGATCG | 1189 |
| | RAGAG | |
| L15:52 | GCCGAACAGGAAGGCTGGCTCTGCACCTTGGTGATCA | 1190 |
| | TTGAG | |
| L15:53 | GGCCTTCAGCTGCTTCTCCAATCCTGATATGATCAGT | 1191 |
| | TCAAG | |
| L15:54 | GCGCCAAGGTACCTTCTGCAGCTATCATTAAGACCCA | 1192 |
| | CTTTC | |

TABLE 7
Sequence summary of top hits from Libraries L10, L11, L12, L13, and L15.

| | Sequence Position/Residue Substitution | | | | | | | | | | | | | | | | | | | | | | | |
|---|---|---|---|---|---|---|---|---|---|---|---|---|---|---|---|---|---|---|---|---|---|---|---|---|
| Clone | 55 | 57 | 60 | 61 | 62 | 64 | 65 | 67 | 68 | 69 | 72 | 73 | 75 | 77 | 82 | 85 | 86 | 88 | 92 | 99 | 100 | 101 | 104 | 105 |
| TetR (B) | L | I | L | D | R | H | T | F | C | P | G | E | W | D | N | S | F | C | S | V | H | L | R | P |
| L10-A04 | M | — | — | — | — | A | — | Y | — | — | — | — | — | — | K | — | M | N | — | — | C | — | G | F |
| L10-A05 | M | — | — | — | — | A | — | Y | — | — | — | — | — | — | K | — | M | — | — | — | C | — | G | F |
| L10-A06 | M | — | — | — | — | A | — | Y | L | — | — | A | — | — | K | — | M | N | — | — | C | — | G | F |
| L10-A09 | M | — | — | — | P | A | I | L | L | — | — | A | — | — | — | — | M | R | — | — | C | — | G | F |
| L10-A11 | M | — | — | — | — | A | — | Y | L | — | — | — | — | — | K | — | M | — | — | — | C | — | G | F |
| L10-B02 | M | — | — | — | P | A | — | — | — | L | — | — | — | — | — | — | M | N | — | — | C | — | G | F |
| L10-B03 | M | — | — | — | — | A | — | Y | S | — | — | — | — | — | K | — | M | — | — | — | C | — | G | F |
| L10-B06 | M | — | — | — | P | A | P | L | L | — | — | A | — | — | — | — | M | — | — | — | C | — | G | F |
| L10-B07 | M | — | — | — | — | A | I | L | L | — | — | — | — | — | — | — | M | — | — | — | C | — | G | F |
| L10-B08 | M | — | — | — | — | A | — | Y | L | — | — | A | — | — | K | — | M | R | — | — | C | — | G | F |
| L11-C02 | M | — | — | — | P | A | — | Y | S | — | — | — | — | — | K | — | M | — | — | — | C | — | G | F |
| L11-C06 | M | — | — | — | — | A | — | Y | S | — | R | — | — | N | — | — | M | N | — | — | C | — | G | F |
| L12-1-10 | M | F | — | — | — | A | I | — | L | — | — | A | — | N | T | — | M | N | — | — | C | — | G | F |
| L12-1-11 | M | F | — | — | P | A | I | Y | L | — | — | — | — | N | H | — | M | N | — | — | C | — | G | F |
| L12-1-21 | M | F | — | — | — | A | P | Y | L | — | — | A | — | N | — | — | M | N | — | — | C | — | G | F |
| L12-2-13 | M | — | — | — | — | A | I | Y | L | — | — | A | — | N | — | — | M | N | — | — | C | — | G | F |
| L12-2-23 | M | F | — | — | — | A | — | Y | L | — | — | — | — | N | — | — | M | N | — | — | C | — | G | F |
| L12-2-27 | M | F | — | — | — | A | I | Y | L | — | — | A | — | N | — | — | M | N | — | L | C | — | G | F |
| L12-2-48 | M | — | — | — | — | A | I | Y | L | — | — | — | — | N | — | — | M | N | — | — | C | — | G | F |
| L13-1-9 | M | — | — | — | — | A | — | Y | — | — | — | — | — | — | K | — | M | N | — | — | A | — | G | F |
| L13-1-10 | M | — | F | — | — | D | — | Y | — | — | — | — | C | — | K | — | M | N | — | — | A | — | G | F |
| L13-1-16 | M | — | F | — | — | K | — | Y | — | — | — | — | — | — | R | — | M | N | — | — | A | — | G | F |
| L13-1-42 | M | — | — | — | — | K | — | Y | — | — | — | — | — | — | K | — | M | N | — | — | A | — | G | F |
| L13-1-43 | M | — | — | — | — | A | — | Y | — | — | — | — | — | — | R | — | M | N | — | — | A | — | G | F |
| L13-2-18 | M | — | F | — | — | A | — | Y | — | — | — | — | — | — | K | — | M | N | R | — | A | — | G | F |
| L13-2-23 | M | — | F | — | — | A | — | Y | — | — | — | — | — | — | K | — | M | N | — | — | A | — | G | F |
| L13-2-24 | M | — | — | — | — | K | — | Y | L | — | — | — | — | — | — | — | M | N | — | — | C | — | G | F |
| L15-1 | M | — | — | — | — | Q | V | N | L | — | — | — | — | — | W | — | M | N | — | — | C | — | G | F |
| L15-14 | M | — | — | — | — | R | I | Y | L | — | — | — | — | — | K | — | M | N | — | — | C | — | G | F |
| L15-20 | M | — | — | — | P | K | I | Y | L | — | — | — | — | — | R | — | M | N | — | — | C | — | G | F |
| L15-35 | M | — | — | — | — | T | — | Y | L | — | — | — | — | N | W | — | M | N | — | — | C | — | G | F |
| L15-36 | M | — | — | G | — | K | — | Y | L | — | — | — | — | — | W | — | M | N | — | — | C | — | G | F |
| L15-41 | M | — | — | — | — | K | — | Y | L | — | — | — | — | — | K | — | M | I | — | — | C | — | G | F |

| | Sequence Position/Residue Substitution | | | | | | | | | | | | | | | | | | | | | | |
|---|---|---|---|---|---|---|---|---|---|---|---|---|---|---|---|---|---|---|---|---|---|---|---|
| Clone | 108 | 113 | 116 | 118 | 121 | 129 | 134 | 135 | 139 | 140 | 144 | 145 | 147 | 148 | 150 | 151 | 153 | 170 | 174 | 177 | 184 | 195 | 203 |
| TetR (B) | K | L | Q | A | C | N | L | S | H | F | C | V | E | D | E | H | V | L | I | F | P | C | C |
| L10-A04 | Q | A | S | — | — | — | M | Q | I | Y | — | — | L | — | — | L | — | V | L | K | — | A | A |
| L10-A05 | Q | A | S | — | — | — | M | Q | I | Y | — | — | L | — | — | L | — | V | L | K | — | S | A |
| L10-A06 | Q | A | S | — | — | — | M | Q | I | Y | — | — | L | — | — | L | — | V | L | K | — | A | S |
| L10-A09 | Q | A | S | — | — | — | M | Q | I | Y | — | — | L | — | — | L | — | V | L | K | — | — | A |
| L10-A11 | Q | A | S | — | T | — | M | Q | I | Y | — | — | L | — | — | L | — | V | L | K | L | R | — |
| L10-B02 | Q | A | S | — | — | — | M | Q | I | Y | — | — | L | — | — | L | — | V | L | K | — | S | — |
| L10-B03 | Q | A | S | — | T | — | M | Q | I | Y | — | — | L | — | — | L | — | V | L | K | — | A | A |
| L10-B06 | Q | A | S | — | T | — | M | Q | I | Y | — | A | L | — | — | L | — | V | L | K | — | S | R |
| L10-B07 | Q | A | R | — | T | — | W | Q | I | Y | — | — | L | — | — | L | — | V | L | K | — | G | S |
| L10-B08 | Q | A | S | — | T | — | M | Q | I | Y | — | — | L | N | — | L | — | V | L | K | — | A | A |
| L10-C02 | Q | A | S | — | — | — | M | Q | I | Y | S | A | L | — | — | L | — | V | L | K | — | R | — |
| L10-C06 | Q | A | S | — | T | — | M | Q | I | Y | — | — | L | — | — | L | — | V | V | K | — | A | — |
| L12-1-10 | R | A | R | — | T | Q | W | Q | I | Y | — | — | L | — | — | L | — | V | W | K | — | S | A |
| L12-1-11 | Q | A | R | — | T | — | W | Q | I | Y | — | — | L | — | — | L | — | V | W | K | — | S | A |
| L12-1-21 | Q | A | H | — | T | — | W | Q | I | Y | — | — | L | — | Q | L | — | V | W | K | — | S | A |
| L12-2-13 | Q | A | S | — | T | Q | W | Q | I | Y | — | — | L | — | — | L | F | V | V | K | — | S | A |
| L12-2-23 | R | A | R | — | T | — | W | Q | I | Y | — | — | L | — | — | L | — | V | W | K | — | S | A |
| L12-2-27 | Q | A | R | — | T | Q | W | Q | I | Y | — | — | L | — | — | L | — | V | W | K | — | S | A |
| L12-2-48 | Q | A | R | — | T | — | W | Q | I | Y | — | — | L | — | — | L | — | V | L | K | — | S | A |
| L13-1-9 | Q | M | S | — | T | — | F | Q | I | Y | — | — | L | — | — | L | — | V | Y | K | — | — | A |
| L13-1-10 | Q | A | S | — | T | — | F | Q | I | Y | — | — | L | — | — | L | — | V | — | H | — | S | — |
| L13-1-16 | Q | M | S | — | T | — | M | Q | I | Y | — | — | L | — | — | L | — | V | Y | K | — | — | A |
| L13-1-42 | Q | M | S | — | T | — | M | Q | I | Y | — | — | L | — | — | L | — | V | Y | K | — | S | — |
| L13-1-43 | Q | M | S | — | T | — | F | Q | I | Y | — | — | L | — | — | L | — | V | Y | K | — | — | — |
| L13-2-18 | Q | A | C | — | T | — | F | Q | I | Y | — | — | L | — | — | L | — | V | — | K | — | — | — |
| L13-2-23 | Q | A | C | — | T | — | F | Q | I | Y | — | — | L | — | — | L | — | V | Y | K | — | — | — |
| L13-2-24 | Q | A | W | — | T | — | F | Q | I | Y | — | — | L | — | — | L | — | V | L | H | — | S | A |
| L15-1 | R | S | K | — | T | — | F | Q | I | Y | — | — | L | — | — | L | — | V | — | A | — | S | A |
| L15-14 | Q | Q | S | — | T | — | N | Q | I | Y | — | — | L | — | — | L | — | V | — | A | — | S | A |
| L15-20 | R | A | T | — | T | — | F | Q | I | Y | — | — | L | — | — | L | — | V | Y | A | — | S | A |
| L15-35 | Q | M | S | — | T | — | M | Q | I | Y | — | — | L | — | — | L | — | V | V | A | — | S | A |
| L15-36 | Q | M | N | D | T | — | M | Q | I | Y | R | — | L | — | — | L | — | V | F | — | — | S | A |
| L15-41 | — | A | T | — | T | — | F | Q | I | Y | — | — | L | — | — | L | — | V | F | A | — | S | A |

Various nucleotide sequences of the top hits from Libraries L10, L11, L12, L13, and L15 are set forth in SEQ ID NOS: 1193-1380. Various amino acid sequences of top hits from Libraries L10, L11, L12, L13, and L15 are set forth in SEQ ID NOS: 1381-1568.

### Example 4

### Chlorsulfuron Repressor Shuffling

### A. Second-Round Shuffling

The original library was designed to thifensulfuron, but once induction activity was established with other SU compounds having potentially better soil and in planta stability properties than the original ligand, the evolution process was re-directed towards these alternative ligands. Of particular interest were herbicides metsulfuron, sulfometuron, ethametsulfuron and chlorsulfuron. For this objective, parental clones L1-9, -22, -29 and -44 were chosen for further shuffling. Clone L1-9 has strong activity on both ethametsulfuron and chlorsulfuron; clone L1-22 has strong sulfometuron activity; clone L1-29 has moderate metsulfuron activity; and clone L1-44 has moderate activity on metsulfuron, ethametsulfuron and chlorsulfuron. (Data not shown.). No clones found in the initial screen were exceptionally reactive to metsulfuron. These four clones were also chosen due to their relatively strong repressor activity, showing low β-gal background activity without inducer. Strong repressor activity is important for establishing a system which is both highly sensitive to the presence of inducer, and tightly off in the absence of inducer.

Based on the sequence information from parental clones L1-9, -22, -29 and -44, two second round libraries were designed, constructed and screened. The first library, L2, consisted of a ‘family’ shuffle whereby the amino acid diversity between the selected parental clones was varied using synthetic assembly of oligonucleotides to find clones improved in responsiveness to any of the four new target ligands. A summary of the diversity used and the resulting hit sequences for library L2 is shown in Table 10.

TABLE 10

| | Amino acid residue position | | | | | | | | | | | |
|---|---|---|---|---|---|---|---|---|---|---|---|---|
| Clone | 60 | 64 | 82 | 86 | 100 | 104 | 105 | 113 | 116 | 134 | 135 | 138 |
| wt | L | H | N | F | H | R | P | L | Q | L | S | G |
| Parents | | | | | | | | | | | | |
| L1-9 | — | A | — | M | C | G | F | A | S | M | Q | C |
| L1-22 | M | — | T | Y | C | A | I | K | N | R | Q | R |
| L1-29 | M | Q | T | M | W | — | W | P | M | W | — | C |
| L1-44 | — | A | — | Y | Y | A | V | A | — | V | K | A |
| Hits | | | | | | | | | | | | |
| L2-2 | — | Q | — | M | C | — | F | K | — | V | — | R |
| L2-9 | M | Q | — | M | Y | — | W | A | — | W | — | A |
| L2-10 | — | A | — | M | W | G | W | K | M | M | — | R |
| L2-13 | — | Q | — | M | C | — | W | A | — | W | Q | R |
| L2-14 | M | A | — | M | C | — | W | A | M | V | — | R |
| L2-18 | M | Q | T | M | W | — | W | A | — | M | — | R |
| L1-45 | A | Q | — | W | W | G | L | P | V | T | Q | R |
| Un- | ran- | ran- | ran- | ran- | W > C, | R >> G, | W > V > | ran- | ran- | ran- | S >> Q, | A >> C, |
| selected | dom | dom | dom | dom | Y | A | I, F | dom | dom | dom | K | R |
| frequency | | | | | | | | | | | | |

| | Amino acid residue position | | | | | | | Inducer |
|---|---|---|---|---|---|---|---|---|
| Clone | 139 | 147 | 151 | 164 | 174 | 177 | 203 | preference |
| wt | H | E | H | D | I | F | C | atc |
| Parents | | | | | | | | |
| L1-9 | I | L | L | — | L | K | — | 4, 9 (weak) |
| L1-22 | V | F | M | — | S | L | S | 3 |
| L1-29 | N | S | R | — | W | S | — | 9 (weak) |
| L1-44 | G | W | S | A | V | A | — | 9 (weak) |
| Hits | | | | | | | | |
| L2-2 | I | W | M | — | W | L | — | 4 (inverse) |
| L2-9 | I | W | S | — | S | K | — | 9 (leaky) |
| L2-10 | I | L | L | — | W | K | — | 4 (leaky) |
| L2-13 | I | S | M | — | V | K | — | 9 |
| L2-14 | V | F | S | A | L | K | — | 9 |
| L2-18 | N | F | L | A | W | K | — | 9 |
| L1-45 | — | G | R | — | A | L | — | 3, 4 |
| Unselected | G, N > | random | random | random | random | random | C >> S | |
| frequency | V > I | | | | | | | |

The oligonucleotides used to construct the library are shown in Table 11. The L2 oligonucleotides were assembled, cloned and screened as per the protocol described for library L1 except that each ligand was tested at 2 ppm to increase the stringency of the assay, which is a 10-fold reduction from 1st round library screening concentration.

TABLE 11

| | | SEQ |
|---|---|---|
| Oligo | Sequence | ID |
| L2:01 | TATTGGCATGTAAAAAATAAGCGAGCTCTGCTCGACGC | 1569 |
| | CTTA | |
| L2:02 | GCCATTGAGATGWTGGATAGGCACCASACTCACTTTTG | 1570 |
| | CCCT | |
| L2:03 | GCCATTGAGATGWTGGATAGGCACGCAACTCACTTTTG | 1571 |
| | CCCT | |
| L2:04 | TTAGAAGGGGAAAGCTGGCAAGATTTTTTACGTAATAM | 1572 |
| | TGCT | |
| L2:05 | AAAAGTTACAGATGTGCTTTACTAAGTCATCGCGATGG | 1573 |
| | AGCA | |
| L2:06 | AAAAGTATGAGATGTGCTTTACTAAGTCATCGCGATGG | 1574 |
| | AGCA | |
| L2:07 | AAAGTATRTTTAGGTACACGCDTCACAGAAAAACAGTA | 1575 |
| | TGAA | |
| L2:08 | AAAGTATRTTTAGGTACACGCTGGACAGAAAAACAGTA | 1576 |
| | TGAA | |
| L2:09 | AAAGTATRTTTAGGTACAGSTDTCACAGAAAAACAGTA | 1577 |
| | TGAA | |
| L2:10 | AAAGTATRTTTAGGTACAGSTTGGACAGAAAAACAGTA | 1578 |
| | TGAA | |
| L2:11 | AAAGTATGGTTAGGTACACGCDTCACAGAAAAACAGTA | 1579 |
| | TGAA | |
| L2:12 | AAAGTATGGTTAGGTACACGCTGGACAGAAAAACAGTA | 1580 |
| | TGAA | |
| L2:13 | AAAGTATGGTTAGGTACAGSTDTCACAGAAAAACAGTA | 1581 |
| | TGAA | |
| L2:14 | AAAGTATGGTTAGGTACAGSTTGGACAGAAAAACAGTA | 1582 |
| | TGAA | |
| L2:15 | ACTAAAGAAAATARCTTAGCCTTTTTATGCCAACAAGG | 1583 |
| | TTTT | |
| L2:16 | ACTAAAGAAAATCAATTAGCCTTTTTATGCCAACAAGG | 1584 |
| | TTTT | |
| L2:17 | ACTAAAGAAAATATGTTAGCCTTTTTATGCCAACAAGG | 1585 |
| | TTTT | |
| L2:18 | ACTSCTGAAAATARCTTAGCCTTTTTATGCCAACAAGG | 1586 |
| | TTTT | |
| L2:19 | ACTSCTGAAAATCAATTAGCCTTTTTATGCCAACAAGG | 1587 |
| | TTTT | |
| L2:20 | ACTSCTGAAAATATGTTAGCCTTTTTATGCCAACAAGG | 1588 |
| | TTTT | |
| L2:21 | TCACTAGAGAATGCATTATATGCARTGAGTGCTGTGGC | 1589 |
| | TAWT | |
| L2:22 | TCACTAGAGAATGCATTATATGCARTGAGTGCTGTGGC | 1590 |
| | TGKT | |
| L2:23 | TCACTAGAGAATGCATTATATGCARTGAGTGCTGTGYG | 1591 |
| | CAWT | |
| L2:24 | TCACTAGAGAATGCATTATATGCARTGAGTGCTGTGYG | 1592 |
| | CGKT | |
| L2:25 | TCACTAGAGAATGCATTATATGCARTGMAAGCTGTGGC | 1593 |
| | TAWT | |
| L2:26 | TCACTAGAGAATGCATTATATGCARTGMAAGCTGTGGC | 1594 |
| | TGKT | |
| L2:27 | TCACTAGAGAATGCATTATATGCARTGMAAGCTGTGYG | 1595 |
| | CAWT | |
| L2:28 | TCACTAGAGAATGCATTATATGCARTGMAAGCTGTGYG | 1596 |
| | CGKT | |
| L2:29 | TCACTAGAGAATGCATTATATGCAWGGAGTGCTGTGGC | 1597 |
| | TAWT | |
| L2:30 | TCACTAGAGAATGCATTATATGCAWGGAGTGCTGTGGC | 1598 |
| | TGKT | |
| L2:31 | TCACTAGAGAATGCATTATATGCAWGGAGTGCTGTGYG | 1599 |
| | CAWT | |
| L2:32 | TCACTAGAGAATGCATTATATGCAWGGAGTGCTGTGYG | 1600 |
| | CGKT | |
| L2:33 | TCACTAGAGAATGCATTATATGCAWGGMAAGCTGTGGC | 1601 |
| | TAWT | |
| L2:34 | TCACTAGAGAATGCATTATATGCAWGGMAAGCTGTGGC | 1602 |
| | TGKT | |
| L2:35 | TCACTAGAGAATGCATTATATGCAWGGMAAGCTGTGYG | 1603 |
| | CAWT | |
| L2:36 | TCACTAGAGAATGCATTATATGCAWGGMAAGCTGTGYG | 1604 |
| | CGKT | |
| L2:37 | TTTACTTTAGGTTGCGTATTGTKGGATCAAGAGAGMCA | 1605 |
| | AGTC | |
| L2:38 | TTTACTTTAGGTTGCGTATTGTKGGATCAAGAGMTGCA | 1606 |
| | AGTC | |
| L2:39 | TTTACTTTAGGTTGCGTATTGTYTGATCAAGAGAGMCA | 1607 |
| | AGTC | |
| L2:40 | TTTACTTTAGGTTGCGTATTGTYTGATCAAGAGMTGCA | 1608 |
| | AGTC | |
| L2:41 | GCTAAAGAAGAAAGGGAAACACCTACTACTGMTAGTAT | 1609 |
| | GCCG | |
| L2:42 | CCATTATTACGACAAGCTAGTGAATTATTGGATCACCA | 1610 |
| | AGGT | |
| L2:43 | CCATTATTACGACAAGCTAGTGAATTAKCAGATCACCA | 1611 |
| | AGGT | |
| L2:44 | CCATTATTACGACAAGCTAGTGAATTAAAGGATCACCA | 1612 |
| | AGGT | |
| L2:45 | CCATTATTACGACAAGCTTKGGAATTATTGGATCACCA | 1613 |
| | AGGT | |
| L2:46 | CCATTATTACGACAAGCTTKGGAATTAKCAGATCACCA | 1614 |
| | AGGT | |
| L2:47 | CCATTATTACGACAAGCTTKGGAATTAAAGGATCACCA | 1615 |
| | AGGT | |
| L2:48 | CCATTATTACGACAAGCTGTAGAATTATTGGATCACCA | 1616 |
| | AGGT | |
| L2:49 | CCATTATTACGACAAGCTGTAGAATTAKCAGATCACCA | 1617 |
| | AGGT | |
| L2:50 | CCATTATTACGACAAGCTGTAGAATTAAAGGATCACCA | 1618 |
| | AGGT | |
| L2:51 | GCAGAGCCAGCCTTCTTATTCGGCCTTGAATTGATCAT | 1619 |
| | ATGC | |
| L2:52 | GGATTAGAAAAACAACTTAAATSCGAAAGTGGGTCT | 1620 |
| | TAA | |
| L2:53 | CCTATCCAWCATCTCAATGGCTAAGGCGTCGAGCAGAG | 1621 |
| | CTCG | |
| L2:54 | TTGCCAGCTTTCCCCTTCTAAAGGGCAAAAGTGAGTST | 1622 |
| | GGTG | |
| L2:55 | TTGCCAGCTTTCCCCTTCTAAAGGGCAAAAGTGAGTTG | 1623 |
| | CGTG | |
| L2:56 | TAAAGCACATCTGTAACTTTTAGCAKTATTACGTAAAA | 1624 |
| | AATC | |
| L2:57 | TAAAGCACATCTCATACTTTTAGCAKTATTACGTAAAA | 1625 |
| | AATC | |
| L2:58 | GCGTGTACCTAAAYATACTTTTGCTCCATCGCGATGAC | 1626 |
| | TTAG | |
| L2:59 | ASCTGTACCTAAAYATACTTTTGCTCCATCGCGATGAC | 1627 |
| | TTAG | |
| L2:60 | GCGTGTACCTAACCATACTTTTGCTCCATCGCGATGAC | 1628 |
| | TTAG | |
| L2:61 | ASCTGTACCTAACCATACTTTTGCTCCATCGCGATGAC | 1629 |
| | TTAG | |
| L2:62 | GGCTAAGYTATTTTCTTTAGTTTCATACTGTTTTTCTG | 1630 |
| | TGAH | |
| L2:63 | GGCTAATTGATTTTCTTTAGTTTCATACTGTTTTTCTG | 1631 |
| | TGAH | |
| L2:64 | GGCTAACATATTTTCTTTAGTTTCATACTGTTTTTCTG | 1632 |
| | TGAH | |
| L2:65 | GGCTAAGYTATTTTCAGSAGTTTCATACTGTTTTTCTG | 1633 |
| | TGAH | |
| L2:66 | GGCTAATTGATTTTCAGSAGTTTCATACTGTTTTTCTG | 1634 |
| | TGAH | |
| L2:67 | GGCTAACATATTTTCAGSAGTTTCATACTGTTTTTCTG | 1635 |
| | TGAH | |
| L2:68 | GGCTAAGYTATTTTCTTTAGTTTCATACTGTTTTTCTG | 1636 |
| | TCCA | |
| L2:69 | GGCTAATTGATTTTCTTTAGTTTCATACTGTTTTTCTG | 1637 |
| | TCCA | |
| L2:70 | GGCTAACATATTTTCTTTAGTTTCATACTGTTTTTCTG | 1638 |
| | TCCA | |
| L2:71 | GGCTAAGYTATTTTCAGSAGTTTCATACTGTTTTTCTG | 1639 |
| | TCCA | |
| L2:72 | GGCTAATTGATTTTCAGSAGTTTCATACTGTTTTTCTG | 1640 |
| | TCCA | |
| L2:73 | GGCTAACATATTTTCAGSAGTTTCATACTGTTTTTCTG | 1641 |
| | TCCA | |
| L2:74 | ATATAATGCATTCTCTAGTGAAAAACCTTGTTGGCATA | 1642 |
| | AAAA | |
| L2:75 | CAATACGCAACCTAAAGTAAAAWTAGCCACAGCACTCA | 1643 |
| | YTGC | |
| L2:76 | CAATACGCAACCTAAAGTAAAAMCAGCCACAGCACTCA | 1644 |
| | YTGC | |
| L2:77 | CAATACGCAACCTAAAGTAAAAWTGCRCACAGCACTCA | 1645 |
| | YTGC | |
| L2:78 | CAATACGCAACCTAAAGTAAAAMCGCRCACAGCACTCA | 1646 |
| | YTGC | |
| L2:79 | CAATACGCAACCTAAAGTAAAAWTAGCCACAGCTTKCA | 1647 |
| | YTGC | |
| L2:80 | CAATACGCAACCTAAAGTAAAAMCAGCCACAGCTTKCA | 1648 |
| | YTGC | |
| L2:81 | CAATACGCAACCTAAAGTAAAAWTGCRCACAGCTTKCA | 1649 |
| | YTGC | |
| L2:82 | CAATACGCAACCTAAAGTAAAAMCGCRCACAGCTTKCA | 1650 |
| | YTGC | |
| L2:83 | CAATACGCAACCTAAAGTAAAAWTAGCCACAGCACTCC | 1651 |
| | WTGC | |
| L2:84 | CAATACGCAACCTAAAGTAAAAMCAGCCACAGCACTCC | 1652 |
| | WTGC | |
| L2:85 | CAATACGCAACCTAAAGTAAAAWTGCRCACAGCACTCC | 1653 |
| | WTGC | |
| L2:86 | CAATACGCAACCTAAAGTAAAAMCGCRCACAGCACTCC | 1654 |
| | WTGC | |
| L2:87 | CAATACGCAACCTAAAGTAAAAWTAGCCACAGCTTKCC | 1655 |
| | WTGC | |
| L2:88 | CAATACGCAACCTAAAGTAAAAMCAGCCACAGCTTKCC | 1656 |
| | WTGC | |
| L2:89 | CAATACGCAACCTAAAGTAAAAWTGCRCACAGCTTKCC | 1657 |
| | WTGC | |
| L2:90 | CAATACGCAACCTAAAGTAAAAMCGCRCACAGCTTKCC | 1658 |
| | WTGC | |
| L2:91 | TGTTTCCCTTTCTTCTTTAGCGACTTGKCTCTCTTGAT | 1659 |
| | CCMA | |
| L2:92 | TGTTTCCCTTTCTTCTTTAGCGACTTGCAKCTCTTGAT | 1660 |
| | CCMA | |
| L2:93 | TGTTTCCCTTTCTTCTTTAGCGACTTGKCTCTCTTGAT | 1661 |
| | CARA | |
| L2:94 | TGTTTCCCTTTCTTCTTTAGCGACTTGCAKCTCTTGAT | 1662 |
| | CARA | |
| L2:95 | ACTAGCTTGTCGTAATAATGGCGGCATACTAKCAGTAG | 1663 |
| | TAGG | |
| L2:96 | CMAAGCTTGTCGTAATAATGGCGGCATACTAKCAGTAG | 1664 |
| | TAGG | |
| L2:97 | TACAGCTTGTCGTAATAATGGCGGCATACTAKCAGTAG | 1665 |
| | TAGG | |
| L2:98 | GAATAAGAAGGCTGGCTCTGCACCTTGGTGATCCAATA | 1666 |
| | ATTC | |
| L2:99 | GAATAAGAAGGCTGGCTCTGCACCTTGGTGATCTGMTA | 1667 |
| | ATTC | |
| L2:100 | GAATAAGAAGGCTGGCTCTGCACCTTGGTGATCCTTTA | 1668 |
| | ATTC | |
| L2:101 | TTTAAGTTGTTTTTCTAATCCGCATATGATCAATTCAA | 1669 |
| | GGCC | |
| L2:102 | GGGAACTTCGGCGCGCCTTAAGACCCACTTTCGSA | 1670 |

### A. Third Round Library Design and Screening

### Library L6: Shuffling for Enhanced Chlorsulfuron Response

Since clones L2-14 and L2-18 had the best chlorsulfuron activity profile from library L2, their amino acid diversity was used as the basis for the next round of shuffling. In addition to the diversity provided by these backbone sequences, additional residue changes thought to enhance packing of chlorsulfuron based on the 3D model predictions were included. New amino acid positions targeted were 67, 109, 112 and 173 (see, Table 12). Substitution of Gln (Q) at position 108 and Val (V) at position 170 were shown to likely be important changes in library L4 for gaining enhanced SU responsiveness and so were varied here as well. A summary of the diversity chose is shown in Table 12. The oligonucleotides designed and used to generate library 6 are shown in Table 13.

Library L6 was assembled, rescued, ligated into pVER7314, transformed into E. coli KM3 and plated out onto LB carbenicillin/kanamycin, and carbenicillin only control media as before. Library plates were then picked into 42 384-well microtiter plates (˜16,000 clones) containing 60 μl LB carbenicillin (Cb) broth per well. After overnight growth at 37° C. the cultures were stamped onto M9 assay plates containing no inducer, 0.2 ppm, and 2.0 ppm chlorsulfuron as test inducer. Following incubation at 30° C. for ˜48 hrs, putative hits responding to chlorsulfuron treatment as determined by increased blue colony color were re-arrayed into six 96-well microtiter plates and used to stamp a fresh set of M9 assay plates to confirm the above results. For a more detailed analysis of the relative induction by chlorsulfuron, digital photographs were taken of the plates after various time points of incubation at 30° C. and colony color intensity measured using the digital image analysis freeware program ImageJ (Rasband, US National Institutes of Health, Bethesda, Md., USA, rsb.info.nih.gov/ij/, 1997-2007). Using these results enabled ranking of clones in multiplex format by background activity (no inducer), activation with low or high level inducer application (blue color with inducer), and fold activation (activation divided by background). Activation studies using 0.2 μg/ml chlorsulfuron as inducer for the top set of clones shows an approximately 3 fold improvement in activation while obtaining lower un-induced levels of expression (Data not shown.) In addition to this analysis, DNA sequence information for most clones (490 clones) was obtained and the deduced polypeptides aligned with each other as well as with their corresponding activity information. From this analysis sequence-activity relationships were derived. (Data not shown.) Residues biased for improved activity are indicated in larger bold type. Briefly, C at position 100, and Q at positions 108 and 109 strongly correlated with activation, while R at position 138, L at position 170, and A or G at position 173 were highly preferred in clones with the lowest background activity. Though some positions were strongly biased, i.e., observed more frequently in the selected population, the entirety of introduced diversity was observed in the full hit population. This information will aid in the design of further libraries to improve responsiveness to chlorsulfuron.

TABLE 12

| | Amino acid residue position | | | | | | | | | | | | | |
|---|---|---|---|---|---|---|---|---|---|---|---|---|---|---|
| | 60 | 64 | 67 | 82 | 86 | 100 | 105 | 108 | 109 | 112 | 113 | 116 | 134 | 138 |
| | | Library Diversity | | | | | | | | | | | | |
| | | A | M | N | | C | | Q | M | S | | M | M | G |
| | | Q | Y | T | | W | | K | L | T | | Q | V | R |
| | | | F | | | | | | Q | A | | | | |
| | | | L | | | | | | H | G | | | | |
| Sequence | | | I | | | | | | | | | | | |
| Name | | | V | | | | | | | | | | | |
| wt reference | L | H | F | N | F | H | P | K | Q | T | L | Q | L | G |
| L2-14 | M | A | F | N | M | C | W | K | Q | T | A | M | V | R |
| L2-18 | M | Q | F | T | M | W | W | K | Q | T | A | Q | M | R |
| L6-1B03 | M | A | I | N | M | C | W | Q | Q | A | A | M | V | R |
| L6-2C09 | M | Q | Y | T | M | C | W | Q | L | T | A | Q | M | R |
| L6-2D07 | M | Q | F | T | M | C | W | Q | Q | T | A | M | M | R |
| L6-3H02 | M | A | Y | T | M | C | W | Q | H | S | A | M | V | R |
| L6-4D10 | M | Q | Y | N | M | C | W | K | Q | S | A | M | V | R |
| L6-5F05 | M | A | I | N | M | C | W | Q | Q | A | A | Q | V | R |
| L6-5G06 | M | Q | Y | N | M | C | W | Q | Q | T | A | Q | V | R |
| L6-5H06 | M | Q | I | N | M | C | W | K | Q | T | A | M | V | R |
| L6-5H12 | M | A | Y | N | M | C | W | K | Q | T | A | Q | M | R |
| L6-6F07 | M | A | L | T | M | C | W | Q | Q | S | A | M | M | R |
| Bias in top | | none | Y | N | | C | | Q | Q | none | | none | V | R |
| population | | | | | | | | | | | | | | |

| | Amino acid residue | | | | | | | | | | | |
|---|---|---|---|---|---|---|---|---|---|---|---|---|
| | position | | | | | | | | | | | |
| | 139 | 147 | 151 | 164 | 170 | 173 | 174 | 177 | 178 | | | |
| | Library Diversity | | | | | | | | | | | |
| | N | | S | | L | G | L | | | | | 0.2 ppm |
| Sequence | V | | L | | A | A | W | | | 0.2 ppm | Control | 48 hr/Control |
| Name | | | | | V | V | | | | 48 hr | 84 hr | 84 hr |
| wt reference | H | E | H | D | L | A | I | F | D | 5.2 | 5.3 | 1.0 |
| L2-14 | V | F | S | A | L | A | L | K | D | 11.8 | 6.6 | 1.8 |
| L2-18 | N | F | L | A | L | A | W | K | D | 5.9 | 5.7 | 1.0 |
| L6-1B03 | V | F | S | A | L | A | W | K | D | 30.0 | 6.6 | 4.6 |
| L6-2C09 | V | F | L | A | L | A | W | K | D | 13.6 | 5.2 | 2.6 |
| L6-2D07 | V | F | S | A | V | A | W | K | D | 20.0 | 5.8 | 3.4 |
| L6-3H02 | V | F | S | A | V | A | W | K | V | 15.8 | 5.6 | 2.8 |
| L6-4D10 | V | F | S | A | L | A | W | K | D | 18.4 | 5.0 | 3.7 |
| L6-5F05 | V | F | L | A | L | A | W | K | D | 22.0 | 5.4 | 4.1 |
| L6-5G06 | V | F | L | A | L | G | W | K | D | 34.4 | 7.0 | 4.9 |
| L6-5H06 | V | F | L | A | V | A | W | K | D | 13.7 | 5.1 | 2.7 |
| L6-5H12 | V | F | L | A | V | A | W | K | D | 23.7 | 5.7 | 4.2 |
| L6-6F07 | V | F | S | A | L | A | W | K | D | 11.6 | 5.1 | 2.3 |
| Bias in top | V | | none | | L | A/G | W | | D | | | |
| population | | | | | | | | | | | | |

TABLE 13

| | | SEQ |
|---|---|---|
| Oligo | Sequence | ID |
| L6:1 | TATTGGCATGTAAAAAATAAGCGAGCTCTGCTCGACG | 1671 |
| | CCTTA | |
| L6:2 | GCAGAGCCAGCCTTCTTATTCGGCCTTGAATTGATCA | 1672 |
| | TATGC | |
| L6:3 | ATATAATGCATTCTCTAGTGAAAAACCTTGTTGGCAT | 1673 |
| | AAAAA | |
| L6:4 | TTTAAGTTGTTTTTCTAATCCGCATATGATCAATTCA | 1674 |
| | AGGCC | |
| L6:5 | TTAGAAGGGGAAAGCTGGCAAGATTTTTTACGTAATA | 1675 |
| | MTGCT | |
| L6:6 | TAAAGCACATCTCATACTTTTAGCAKTATTACGTAAA | 1676 |
| | AAATC | |
| L6:7 | TTGCCAGCTTTCCCCTTCTAAAGGGCAMAHGTGAGTT | 1677 |
| | GCGTG | |
| L6:8 | TTGCCAGCTTTCCCCTTCTAAAGGGCAATAGTGAGTT | 1678 |
| | GCGTG | |
| L6:9 | GAATAAGAAGGCTGGCTCTGCACCTTGGTGATCCTTT | 1679 |
| | AATTC | |
| L6:10 | GCCATTGAGATGATGGATAGGCACGCAACTCACTATT | 1680 |
| | GCCCT | |
| L6:11 | RSTGCTGAAAATATGTTAGCCTTTTTATGCCAACAAG | 1681 |
| | GTTTT | |
| L6:12 | TTTACTTTAGGTTGCGTATTGTTTGATCAAGAGCTCC | 1682 |
| | AAGTC | |
| L6:13 | TGTTTCCCTTTCTTCTTTAGCGACTTGGAGCTCTTGA | 1683 |
| | TCAAA | |
| L6:14 | GCCATTGAGATGATGGATAGGCACGCAACTCACDTKT | 1684 |
| | GCCCT | |
| L6:15 | GCCATTGAGATGATGGATAGGCACCAAACTCACDTKT | 1685 |
| | GCCCT | |
| L6:16 | GCCATTGAGATGATGGATAGGCACCAAACTCACTATT | 1686 |
| | GCCCT | |
| L6:17 | AAAAGTATGAGATGTGCTTTACTAAGCCATCGCGATG | 1687 |
| | GAGCA | |
| L6:18 | AAAGTATGKTTAGGTACACGCTGGACAGAAMAACAWT | 1688 |
| | ATGAA | |
| L6:19 | AAAGTATGKTTAGGTACACGCTGGACAGAAMAAWTGT | 1689 |
| | ATGAA | |
| L6:20 | RSTGCTGAAAATCAATTAGCCTTTTTATGCCAACAAG | 1690 |
| | GTTTT | |
| L6:21 | TCACTAGAGAATGCATTATATGCARTGAGTGCGTGGR | 1691 |
| | GGGTG | |
| L6:22 | TCACTAGAGAATGCATTATATGCARTGAGTGCGTGGR | 1692 |
| | GGAAC | |
| L6:23 | TTTACTTTAGGTTGCGTATTGTTTGATCAAGAGAGCC | 1693 |
| | AAGTC | |
| L6:24 | GCTAAAGAAGAAAGGGAAACACCTACTACTGCTAGTA | 1694 |
| | TGCCG | |
| L6:25 | CCATTAKTGCGACAAGBTTKGGAATTAAAGGATCACC | 1695 |
| | AAGGT | |
| L6:26 | CCATTAGCCCGACAAGBTTKGGAATTAAAGGATCACC | 1696 |
| | AAGGT | |
| L6:27 | GGATTAGAAAAACAACTTAAATGCGAAAGTGGGTCTT | 1697 |
| | AA | |
| L6:28 | CCTATCCATCATCTCAATGGCTAAGGCGTCGAGCAGA | 1698 |
| | GCTCG | |
| L6:29 | TTGCCAGCTTTCCCCTTCTAAAGGGCAMAHGTGAGTT | 1699 |
| | TGGTG | |
| L6:30 | TTGCCAGCTTTCCCCTTCTAAAGGGCAATAGTGAGTT | 1700 |
| | TGGTG | |
| L6:31 | GCGTGTACCTAAMCATACTTTTGCTCCATCGCGATGG | 1701 |
| | CTTAG | |
| L6:32 | GGCTAACATATTTTCAGCASYTTCATAWTGTTKTTCT | 1702 |
| | GTCCA | |
| L6:33 | GGCTAATTGATTTTCAGCASYTTCATAWTGTTKTTCT | 1703 |
| | GTCCA | |
| L6:34 | GGCTAACATATTTTCAGCASYTTCATACAWTTKTTCT | 1704 |
| | GTCCA | |
| L6:35 | GGCTAATTGATTTTCAGCASYTTCATACAWTTKTTCT | 1705 |
| | GTCCA | |
| L6:36 | CAATACGCAACCTAAAGTAAACACCCYCACAGCACTC | 1706 |
| | AYTGC | |
| L6:37 | CAATACGCAACCTAAAGTAAAGTTCCYCACAGCACTC | 1707 |
| | AYTGC | |
| L6:38 | TGTTTCCCTTTCTTCTTTAGCGACTTGGCTCTCTTGA | 1708 |
| | TCAAA | |
| L6:39 | CMAAVCTTGTCGCAMTAATGGCGGCATACTAGCAGTA | 1709 |
| | GTAGG | |
| L6:40 | CMAAVCTTGTCGGGCTAATGGCGGCATACTAGCAGTA | 1710 |
| | GTAGG | |
| L6:41 | GGGAACTTCGGCGCGCCTTAAGACCCACTTTCGCA | 1711 |

### B. Fourth Round Shuffling

Library L8 Construction and Screening.

Fourth round shuffling incorporated the best diversity from Rd3 shuffling (BB1860) as well as computational diversity (Table 14). The fully synthetic library was constructed from oligonucleotides shown in Tables 15A and 15B. As diversity was very high the library oligo mix was spiked into the parental hit variant oligo mix (5, 10, and 25% mixes) to titer down the number of residue changes per clone. In addition, to varying residues for Cs activity, seven residues (C68, C86, C88, C121, C144, C195, and C203) were varied with TetR family phylogenetic substitutions in an attempt to reduce the number of cysteine residues in the repressor. The PCR assembled libraries were cloned SacI/AscI into pVER7334. This plasmid encodes P_{BAD} promoter controlled expression of a plant optimized TetR DNA binding domain fused to the wt ligand binding domain of TetR(B) encoded by native Tn10 sequence on a SacI to AscI fragment. Approximately 15,000 clones were screened for blue colony color on the M9 XgaI assay plates+/−200 ppb Chlorsulfuron (Cs). Clones were ranked by ratio of color with inducer after 24 hrs incubation over colony color without inducer for 48 hrs of incubation. The sequence trend in the overall larger population of hits (first re-array) was that L55, R104, W105 and L170 were maintained while the C144A substitution was highly preferred. Sequence trends within the hit population were then noted with respect to repression, induction and fold induction (which corrects for leakiness). For repression C68L and C144A are favored in the highly repressed population: 57% and 93% in the top 40 repressed clones vs. 35% and 66% for the remaining 209 clones, respectively. the sequence analysis reveals that substitutions V134L and S135 to E, D, T, or Q were overrepresented. A sequence alignment of the top 20 clones is shown in Table 16.

TABLE 14
Library diversity summary for fourth, fifth and sixth round Chlorsulfuron
repressor shuffling.

| Sequence | TetR(B) | | | |
|---|---|---|---|---|
| position | Sequence | L8 | CsL3 | CsL4.2 |
| 55 | L | M [symbol] | — | — |
| 60 | L | ML | HMN | [symbol] M |
| 64 | H | QILV (SEQ ID | G [symbol] S | G [symbol] |
| | | NO: 2129) | | |
| 67 | F | Y | | — |
| 68 | C | LSC | L | L |
| 78 | F | — | — | [symbol] Y |
| 82 | N | NLIV (SEQ ID | Y [symbol] | FY |
| | | NO: 2130) | | |
| 86 | F | WFYILMC (SEQ | [symbol] S | M |
| | | ID NO: 2131) | | |
| 88 | C | RNC | R | CLR |
| 100 | H | WMVC (SEQ ID | AS | AS |
| | | NO: 2132) | | |
| 104 | R | A [symbol] G | R | R |
| 105 | P | L [symbol] FY (SEQ ID | W | W |
| | | NO: 2133) | | |
| 108 | K | Q | — | — |
| 112 | T | ST | — | — |
| 113 | L | AV | [symbol] G | A |
| 116 | Q | M | L [symbol] | M |
| 117 | L | ML | — | — |
| 121 | C | TC | T | T |
| 131 | L | ML | — | — |
| 134 | L | IV [symbol] | LTV | T |
| 135 | S | AC [symbol] K [symbol] RS [symbol] | DG [symbol] | DS |
| | | (SEQ ID NO: 2134) | | |
| 137 | V | AV | — | — |
| 138 | G | R | H [symbol] | R |
| 139 | H | IV | IV | V |
| 144 | C | W [symbol] C | A | A |
| 147 | E | LGKCRFWV | [symbol] V | [symbol] Q |
| | | (SEQ ID | | |
| | | NO: 2135) | | |
| 151 | H | S | GQS | G [symbol] |
| 155 | K | — | — | KN |
| 163 | T | — | — | PT |
| 165 | S | — | — | RS |
| 170 | L | I [symbol] | L | — |
| 173 | A | — | — | — |
| 174 | I | W | W | W |
| 177 | F | QK | K | K |
| 178 | D | — | — | DE [symbol] |
| 195 | C | SRAC (SEQ ID | A | A |
| | | NO: 2128) | | |
| 203 | C | SRAC (SEQ ID | R | R |
| | | NO: 2128) | | |

TABLE 15A
Library L8 assembly oligonucleotides

| Oligo | SEQ ID NO | Sequence | Group |
|---|---|---|---|
| L8:1 | 1712 | CACACAGGAATCCATGGCCAGACTCGACAAGAGCAAGGTG | 1 |
| L8:2 | 1713 | ATCAACAGCGCACTGGAGCTGCTGAACGAGGTCGGAATCGAA | 2 |
| L8:3 | 1714 | GGCCTCACAACCCGTAAACTCGCCCAGAAGCTCGGGGTAGAG | 3 |
| L8:4 | 1715 | CAGCCTACATTGTATTGGCACGTCAAGAACAAGCGAGCTCTG | 4 |
| L8:5 | 1716 | CTAGACGCCWTGGCCATTGAGATGWTGGATAGGCACCAWACC | 5 |
| L8:6 | 1717 | CTAGACGCCWTGGCCATTGAGATGWTGGATAGGCACVTTACC | |
| L8:7 | 1718 | CACTACTGCCCTTTGGAAGGGGAAAGCTGGCAAGACTTCTTG | 6 |
| L8:8 | 1719 | AGGAACAACGCTAAGAGCWTSAGATGTGCTTTGCTCAGTCAC | 7 |
| L8:9 | 1720 | AGGAACAACGCTAAGAGCTGGAGATGTGCTTTGCTCAGTCAC | |
| L8:10 | 1721 | AGGAACAACGCTAAGAGCTACAGATGTGCTTTGCTCAGTCAC | |
| L8:11 | 1722 | AGGAACVTTGCTAAGAGCWTSAGATGTGCTTTGCTCAGTCAC | |
| L8:12 | 1723 | AGGAACVTTGCTAAGAGCTGGAGATGTGCTTTGCTCAGTCAC | |
| L8:13 | 1724 | AGGAACVTTGCTAAGAGCTACAGATGTGCTTTGCTCAGTCAC | 8 |
| L8:14 | 1725 | CGTGATGGAGCCAAGGTCTGSCTAGGTACAGCGTKGACGGAG | |
| L8:15 | 1726 | CGTGATGGAGCCAAGGTCTGSCTAGGTACAGCGTWCACGGAG | |
| L8:16 | 1727 | CGTGATGGAGCCAAGGTCTGSCTAGGTACASGGTKGACGGAG | |
| L8:17 | 1728 | CGTGATGGAGCCAAGGTCTGSCTAGGTACASGGTWCACGGAG | |
| L8:18 | 1729 | CGTGATGGAGCCAAGGTCRTGCTAGGTACAGCGTKGACGGAG | |
| L8:19 | 1730 | CGTGATGGAGCCAAGGTCRTGCTAGGTACAGCGTWCACGGAG | |
| L8:20 | 1731 | CGTGATGGAGCCAAGGTCRTGCTAGGTACASGGTKGACGGAG | |
| L8:21 | 1732 | CGTGATGGAGCCAAGGTCRTGCTAGGTACASGGTWCACGGAG | |
| L8:22 | 1733 | CAACAGTATGAAWCTGYGGAGAACATGWTGGCCTTCCTGTGC | 9 |
| L8:23 | 1734 | CAACAAGGTTTCTCCCTTGAGAATGCCWTGTACGCAVTCDCG | 10 |
| L8:24 | 1735 | CAACAAGGTTTCTCCCTTGAGAATGCCWTGTACGCAVTCMAG | |
| L8:25 | 1736 | CAACAAGGTTTCTCCCTTGAGAATGCCWTGTACGCAVTCYGC | |
| L8:26 | 1737 | CAACAAGGTTTCTCCCTTGAGAATGCCWTGTACGCAVTCGAM | |
| L8:27 | 1738 | GCTGYGCGGRTTTTCACTCTGGGTTGCGTATTGBKGGATCAA | 11 |
| L8:28 | 1739 | GCTGYGCGGRTTTTCACTCTGGGTTGCGTATTGAAGGATCAA | |
| L8:29 | 1740 | GCTGYGCGGRTTTTCACTCTGGGTTGCGTATTGTKTGATCAA | |
| L8:30 | 1741 | GAGTCCCAAGTCGCTAAGGAGGAGAGGGAAACACCTACTACT | 12 |
| L8:31 | 1742 | GATAGTATGCCGCCACTGMTTCGACAAGCTTGGGAACTCMAA | 13 |
| L8:32 | 1743 | GATCACCAAGGTGCAGAGCCAGCCTTCCTGTTCGGCCTTGAA | 14 |
| L8:33 | 1744 | TTGATCATATGCGGATTGGAGAAGCAGCTGAAGTGTGAAAGT | 15 |
| L8:34 | 1745 | GGGTCTTAAGGCGCGCCGAAGTTCCC | 16 |
| L8:35 | 1746 | CAGCTCCAGTGCGCTGTTGATCACCTTGCTCTTGTCGAGTCT | 17 |
| L8:36 | 1747 | GAGTTTACGGGTTGTGAGGCCTTCGATTCCGACCTCGTTCAG | 18 |
| L8:37 | 1748 | GTGCCAATACAATGTAGGCTGCTCTACCCCGAGCTTCTGGGC | 19 |
| L8:38 | 1749 | CTCAATGGCCAWGGCGTCTAGCAGAGCTCGCTTGTTCTTGAC | 20 |
| L8:39 | 1750 | CCCTTCCAAAGGGCAGTAGTGGGTWTGGTGCCTATCCAWCAT | 21 |
| L8:40 | 1751 | CCCTTCCAAAGGGCAGTAGTGGGTAABGTGCCTATCCAWCAT | |
| L8:41 | 1752 | SAWGCTCTTAGCGTTGTTCCTCAAGAAGTCTTGCCAGCTTTC | 22 |
| L8:42 | 1753 | CCAGCTCTTAGCGTTGTTCCTCAAGAAGTCTTGCCAGCTTTC | |
| L8:43 | 1754 | GTAGCTCTTAGCGTTGTTCCTCAAGAAGTCTTGCCAGCTTTC | |
| L8:44 | 1755 | SAWGCTCTTAGCAABGTTCCTCAAGAAGTCTTGCCAGCTTTC | |
| L8:45 | 1756 | CCAGCTCTTAGCAABGTTCCTCAAGAAGTCTTGCCAGCTTTC | |
| L8:46 | 1757 | GTAGCTCTTAGCAABGTTCCTCAAGAAGTCTTGCCAGCTTTC | |
| L8:47 | 1758 | SCAGACCTTGGCTCCATCACGGTGACTGAGCAAAGCACATCT | 23 |
| L8:48 | 1759 | CAYGACCTTGGCTCCATCACGGTGACTGAGCAAAGCACATCT | |
| L8:49 | 1760 | CTCCRCAGWTTCATACTGTTGCTCCGTCMACGCTGTACCTAG | 24 |
| L8:50 | 1761 | CTCCRCAGWTTCATACTGTTGCTCCGTGWACGCTGTACCTAG | |
| L8:51 | 1762 | CTCCRCAGWTTCATACTGTTGCTCCGTCMACCSTGTACCTAG | |
| L8:52 | 1763 | CTCCRCAGWTTCATACTGTTGCTCCGTGWACCSTGTACCTAG | |
| L8:53 | 1764 | CTCAAGGGAGAAACCTTGTTGGCACAGGAAGGCCAWCATGTT | 25 |
| L8:54 | 1765 | CAGAGTGAAAAYCCGCRCAGCCGHGABTGCGTACAWGGCATT | 26 |
| L8:55 | 1766 | CAGAGTGAAAAYCCGCRCAGCCTKGABTGCGTACAWGGCATT | |
| L8:56 | 1767 | CAGAGTGAAAAYCCGCRCAGCGCRGABTGCGTACAWGGCATT | |
| L8:57 | 1768 | CAGAGTGAAAAYCCGCRCAGCKTCGABTGCGTACAWGGCATT | |
| L8:58 | 1769 | CTCCTTAGCGACTTGGGACTCTTGATCCMVCAATACGCAACC | 27 |
| L8:59 | 1770 | CTCCTTAGCGACTTGGGACTCTTGATCCTTCAATACGCAACC | |
| L8:60 | 1771 | CTCCTTAGCGACTTGGGACTCTTGATCAMACAATACGCAACC | |
| L8:61 | 1772 | AAKCAGTGGCGGCATACTATCAGTAGTAGGTGTTTCCCTCTC | 28 |
| L8:62 | 1773 | TGGCTCTGCACCTTGGTGATCTTKGAGTTCCCAAGCTTGTCG | 29 |
| L8:63 | 1774 | CTCCAATCCGCATATGATCAATTCAAGGCCGAACAGGAAGGC | 30 |
| L8:64 | 1775 | CTTCGGCGCGCCTTAAGACCCACTTTCACACTTCAGCTGCTT | 31 |

TABLE 15B
Oligonucleotide mixes encoding parent clone for library L8.

| | SEQ ID | | |
|---|---|---|---|
| Oligo | NO | Oligo Sequence | Group |
| L6-4010:01 | 1776 | CAGCCTACATTGTATTGGCACGTCAAGAACAAGCGAGCTCTG | 4 |
| L6-4010:02 | 1777 | CTAGACGCCTTGGCCATTGAGATGATGGATAGGCACCAAACC | 5 |
| L6-4010:03 | 1778 | CACTACTYGCCTTTGGAAGGGGAAAGCTGGCAAGACTTCTTG | 6 |
| L6-4010:04 | 1779 | AGGAACAACGCTAAGAGCTGCAGACGTGCTTTGCTCAGTCAC | 7 |
| L6-4010:05 | 1780 | AGGAACAACGCTAAGAGCTGCAGAAATGCTTTGCTCAGTCAC | |
| L6-4010:06 | 1781 | CGTGATGGAGCCAAGGTCTGCCTAGGTACACGGTGGACGGAG | 8 |
| L6-4D10:07 | 1782 | CAACAGTATGAATCTGCGGAGAACATGTTGGCCTTCCTGACC | 9 |
| L6-4010:08 | 1783 | CAACAAGGTTTCTCCCTTGAGAATGCCTTGTACGCAGTCTCC | 10 |
| L6-4010:09 | 1784 | GCTGTGCGGGTTTTCACTCTGGGTTGGGTATTGTTCGATCAA | 11 |
| L6-4010:10 | 1785 | GCTGTGCGGGTTTTCACTCTGGGTGCCGTATTGTTCGATCAA | |
| L6-4010:11 | 1786 | GAGTCCCAAGTCGCTAAGGAGGAGAGGGAAACACCTACTACT | 12 |
| L6-4010:12 | 1787 | GATAGTATGCCGCCACTGCTTCGACAAGCTTGGGAACTCAAA | 13 |
| L6-4010:13 | 1788 | GATCACCAAGGTGCAGAGCCAGCCTTCCTGTTCGGCCTTGAA | 14 |
| L6-4D10:14 | 1789 | TTGATCATAKCCGGATTGGAGAAGCAGCTGAAGKCAGAAAGT | 15 |
| L6-4010:15 | 1790 | TTGATCATAKCCGGATTGGAGAAGCAGCTGAAGAGAGAAAGT | |
| L6-4010:16 | 1791 | TTGATCATACGCGGATTGGAGAAGCAGCTGAAGKCAGAAAGT | |
| L6-4010:17 | 1792 | TTGATCATACGCGGATTGGAGAAGCAGCTGAAGAGAGAAAGT | |
| L6-4010:18 | 1793 | GGGTCTTAATGATAGCTGCAGAAGGTACCTTGGCGCGCC | 16 |
| L6-4010:19 | 1794 | CTCAATGGCCAAGGCGTCTAGCAGAGCTCGCTTGTTCTTGAC | 20 |
| L6-4010:20 | 1795 | CCCTTCCAAAGGCRAGTAGTGGGTTTGGTGCCTATCCATCAT | 21 |
| L6-4010:21 | 1796 | GCAGCTCTTAGCGTTGTTCCTCAAGAAGTCTTGCCAGCTTTC | 22 |
| L6-4010:22 | 1797 | GCAGACCTTGGCTCCATCACGGTGACTGAGCAAAGCACGTCT | 23 |
| L6-4010:23 | 1798 | GCAGACCTTGGCTCCATCACGGTGACTGAGCAAAGCATTTCT | |
| L6-4010:24 | 1799 | CTCCGCAGATTCATACTGTTGCTCCGTCCACCGTGTACCTAG | 24 |
| L6-4010:25 | 1800 | CTCAAGGGAGAAACCTTGTTGGGTCAGGAAGGCCAACATGTT | 25 |
| L6-4010:26 | 1801 | CAGAGTGAAAACCCGCACAGCGGAGACTGCGTACAAGGCATT | 26 |
| L6-4010:27 | 1802 | CTCCTTAGCGACTTGGGACTCTTGATCGAACAATACCCAACC | 27 |
| L6-4010:28 | 1803 | CTCCTTAGCGACTTGGGACTCTTGATCGAACAATACGGCACC | |
| L6-4010:29 | 1804 | AAGCAGTGGCGGCATACTATCAGTAGTAGGTGTTTCCCTCTC | 28 |
| L6-4010:30 | 1805 | TGGCTCTGCACCTTGGTGATCTTTGAGTTCCCAAGCTTGTCG | 29 |
| L6-4D10:31 | 1806 | CTCCAATCCGGMTATGATCAATTCAAGGCCGAACAGGAAGGC | 30 |
| L6-4010:32 | 1807 | CTCCAATCCGCGTATGATCAATTCAAGGCCGAACAGGAAGGC | |
| L6-4010:33 | 1808 | CTGCAGCTATCATTAAGACCCACTTTCTGMCTTCAGCTGCTT | 31 |
| L6-4010:34 | 1809 | CTGCAGCTATCATTAAGACCCACTTTCTCTCTTCAGCTGCTT | |

TABLE 16
Sequence alignment and relative performance of the top 20 L8 hits relative
to parent clone L6-4D10.

| | Colony Assay | | | | | | | | | | | | | | | | |
|---|---|---|---|---|---|---|---|---|---|---|---|---|---|---|---|---|---|
| | Results | | | Residue and Sequence Position | | | | | | | | | | | | | |
| Clone | IND | REP | F. IND | 60 | 64 | 67 | 68 | 86 | 88 | 90 | 100 | 105 | 108 | 112 | 113 | 116 | 121 |
| TetR | ND | ND | ND | L | H | F | — | F | — | — | H | P | — | T | L | Q | — |
| L6-4D10 | 0.2 | 0.6 | 0.4 | M | Q | Y | C | C | C | L | C | W | K | S | A | M | C |
| L8-3F09 | 5.6 | 0.6 | 9.7 | — | — | — | S | L | — | — | — | — | Q | — | — | — | T |
| L8-1A04 | 12.2 | 2.0 | 6.2 | — | — | — | L | C | N | — | — | — | Q | T | — | — | T |
| L8-3B08 | 13.0 | 2.1 | 6.1 | — | — | — | S | L | — | — | — | — | Q | — | — | — | T |
| L8-1B12 | 12.5 | 2.4 | 5.1 | — | — | — | S | W | — | — | — | — | Q | — | — | — | T |
| L8-3D03 | 5.9 | 1.2 | 4.9 | — | — | — | L | C | N | — | — | — | Q | — | — | — | T |
| L8-2F12 | 2.7 | 0.7 | 3.6 | — | — | — | S | C | N | — | — | — | Q | — | — | — | T |
| L8-3F02 | 3.4 | 1.0 | 3.5 | — | — | — | S | C | R | — | — | — | Q | — | — | — | — |
| L8-3E05 | 1.4 | 0.4 | 3.4 | — | — | — | — | L | — | — | — | — | Q | — | — | — | T |
| L8-3A05 | 0.3 | 0.1 | 3.3 | — | — | — | S | C | N | — | — | — | Q | — | — | — | T |
| L8-3A04 | 0.5 | 0.1 | 3.3 | — | — | — | S | C | R | — | — | — | Q | — | — | — | T |
| L8-1A03 | 8.6 | 2.8 | 3.1 | — | — | — | S | C | N | — | — | — | Q | — | — | — | T |
| L8-3F01 | 1.7 | 0.6 | 3.0 | — | — | — | L | C | R | — | — | — | Q | T | — | — | T |
| L8-3A07 | 0.7 | 0.2 | 2.9 | — | — | — | S | M | — | — | — | — | Q | — | — | — | T |
| L8-1A06 | 2.1 | 0.8 | 2.7 | — | — | — | S | C | R | V | — | — | Q | — | — | — | T |
| L8-2H01 | 12.9 | 4.8 | 2.7 | — | — | — | S | C | R | — | — | — | Q | — | — | — | T |
| L8-3F08 | 1.5 | 0.6 | 2.7 | — | — | — | S | C | N | — | V | — | Q | — | — | — | T |
| L8-3A06 | 0.3 | 0.1 | 2.6 | — | — | — | L | C | N | — | — | — | Q | — | — | — | T |
| L8-1E04 | 1.5 | 0.6 | 2.5 | — | — | — | L | C | N | — | — | — | Q | — | — | — | T |
| L8-1A05 | 10.8 | 4.4 | 2.5 | — | — | — | S | C | R | — | — | — | Q | — | — | — | T |
| L8-3B03 | 0.6 | 0.3 | 2.4 | — | — | — | L | C | R | — | — | — | Q | — | — | — | T |

| | Residue and Sequence Position | | | | | | | | | | | | | | |
|---|---|---|---|---|---|---|---|---|---|---|---|---|---|---|---|
| Clone | 131 | 134 | 135 | 137 | 138 | 139 | 144 | 147 | 151 | 164 | 174 | 177 | 195 | 203 | 205 |
| TetR | — | L | — | — | G | H | — | E | H | D | I | F | — | — | — |
| L6-4D10 | L | V | S | V | R | V | C | F | S | A | W | K | C | C | S |
| L8-3F09 | — | L | T | — | — | — | W | — | — | D | — | — | A | R | — |
| L8-1A04 | — | L | E | A | — | — | A | — | — | D | — | — | S | R | — |
| L8-3B08 | — | — | D | A | — | — | W | — | — | D | — | — | R | R | — |
| L8-1B12 | — | L | E | — | — | — | W | — | — | D | — | — | A | A | — |
| L8-3D03 | — | L | D | — | — | — | A | — | — | D | — | — | R | S | — |
| L8-2F12 | — | L | Q | — | — | — | W | — | — | D | — | — | R | S | — |
| L8-3F02 | — | — | — | — | — | I | A | — | — | D | — | — | A | R | — |
| L8-3E05 | — | L | Q | — | — | I | — | — | — | D | — | — | R | R | — |
| L8-3A05 | — | — | — | — | — | — | A | — | — | D | — | — | S | S | — |
| L8-3A04 | — | — | — | — | — | — | A | — | — | D | — | — | R | R | C |
| L8-1A03 | — | L | D | — | — | — | A | — | — | D | — | — | A | R | — |
| L8-3F01 | — | — | — | — | — | — | A | — | — | D | — | — | A | R | — |
| L8-3A07 | — | — | — | — | — | — | W | — | — | D | — | — | A | R | — |
| L8-1A06 | — | — | — | — | — | — | A | — | — | D | — | — | A | R | — |
| L8-2H01 | — | L | E | — | — | — | A | — | — | D | — | — | R | R | — |
| L8-3F08 | M | L | E | A | — | I | W | — | — | D | — | — | R | S | — |
| L8-3A06 | — | — | — | — | — | — | A | — | — | D | — | — | S | R | — |
| L8-1E04 | — | — | — | — | — | — | A | Y | — | D | — | — | R | A | — |
| L8-1A05 | — | — | D | — | — | — | A | — | — | D | — | — | A | A | — |
| L8-3B03 | — | — | — | — | — | — | A | — | — | D | — | — | R | S | — |

Clones ranked by blue colony color intensity thru ImageJ analysis.
IND = induction with 200 ppb Cs at 24 hrs
REP = repression measured without inducer after 48 hrs
F. IND = fold induction: induction with 200 ppb Cs at 24 hrs/repression at 48 hrs

### C. Fifth Round Chlorsulfuron Repressor Shuffling

Saturation mutagenesis of ligand binding pocket: To generate novel diversity for further rounds of shuffling residues 60, 64, 82, 86, 100, 104, 105, 113, 116, 134, 135, 138, 139, 147, 151, 174, and 177 in L8 hit L8-3F01 were subjected to NNK substitution mutagenesis with the following primers shown in Table 17.

TABLE 17
Oligonucleotides used for saturation mutagenesis of putative ligand binding
pocket residues.

| | Residue/ | | SEQ ID |
|---|---|---|---|
| Oligo | Strand | Sequence | NO |
| 3F1-60T | 60 top | CCTTGGCCATTGAGATGNNKGATAGGCACCAAACCCACTAC | 1810 |
| 3F1-60B | 60 bottom | GTAGTGGGTTTGGTGCCTATCMNNCATCTCAATGGCCAAGG | 1811 |
| 3F1-64T | 64 top | GAGATGATGGATAGGCACNNKACCCACTACTTGCCTTTG | 1812 |
| 3F1-64B | 64 bottom | CAAAGGCAAGTAGTGGGTMNNGTGCCTATCCATCATCTC | 1813 |
| 3F1-82T | 60 top | CAAGACTTCTTGAGGAACNNKGCTAAGAGCTGCAGACGTG | 1814 |
| 3F1-82B | 82 bottom | CACGTCTGCAGCTCTTAGCMNNGTTCCTCAAGAAGTCTTG | 1815 |
| 3F1-86T | 86 top | GAGGAACAACGCTAAGAGCNNKAGACGTGCTTTGCTCAGTC | 1816 |
| 3F1-86B | 86 bottom | GACTGAGCAAAGCACGTCTMNNGCTCTTAGCGTTGTTCCTC | 1817 |
| 3F1-100T | 100 top | CGTGATGGAGCCAAGGTCNNKCTAGGTACACGGTGGACG | 1818 |
| 3F1-100B | 100 bottom | CGTCCACCGTGTACCTAGMNNGACCTTGGCTCCATCACG | 1819 |
| 3F1-104T | 104 top | CAAGGTCTGCCTAGGTACANNKTGGACGGAGCAACAGTATG | 1820 |
| 3F1-104B | 104 bottom | CATACTGTTGCTCCGTCCAMNNTGTACCTAGGCAGACCTTG | 1821 |
| 3F1-105T | 105 top | GTCTGCCTAGGTACACGGNNKACGGAGCAACAGTATGAAAC | 1822 |
| 3F1-105B | 105 bottom | GTTTCATACTGTTGCTCCGTMNNCCGTGTACCTAGGCAGAC | 1823 |
| 3F1-113T | 113 top primer | GAGCAACAGTATGAAACTNNKGAGAACATGTTGGCCTTCC | 1824 |
| 3F1-113B | 113 bottom | GGAAGGCCAACATGTTCTCMNNAGTTTCATACTGTTGCTC | 1825 |
| 3F1-116T | 116 top | GTATGAAACTGCGGAGAACNNKTTGGCCTTCCTGACCCAAC | 1826 |
| 3F1-116B | 116 bottom | GTTGGGTCAGGAAGGCCAAMNNGTTCTCCGCAGTTTCATAC | 1827 |
| 3F1-134T | 134 top | GAGAATGCCTTGTACGCANNKTCCGCTGTGCGGGTTTTCAC | 1828 |
| 3F1-134B | 134 bottom | GTGAAAACCCGCACAGCGGAMNNTGCGTACAAGGCATTCTC | 1829 |
| 3F1-135T | 135 top | GAATGCCTTGTACGCAGTCNNKGCTGTGCGGGTTTTCACTC | 1830 |
| 3F1-135B | 135 bottom | GAGTGAAAACCCGCACAGCMNNGACTGCGTACAAGGCATTC | 1831 |
| 3F1-138T | 138 top | GTACGCAGTCTCCGCTGTGNNKGTTTTCACTCTGGGTGCC | 1832 |
| 3F1-138B | 138 bottom | GGCACCCAGAGTGAAAACMNNCACAGCGGAGACTGCGTAC | 1833 |
| 3F1-139T | 139 top | ACGCAGTCTCCGCTGTGCGGNNKTTCACTCTGGGTGCCGTA | 1834 |
| 3F1-139B | 139 bottom | TACGGCACCCAGAGTGAAMNNCCGCACAGCGGAGACTGCGT | 1835 |
| 3F1-147T | 147 top | CACTCTGGGTGCCGTATTGNNKGATCAAGAGTCCCAAGTC | 1836 |
| 3F1-147B | 147 bottom | GACTTGGGACTCTTGATCMNNCAATACGGCACCCAGAGTG | 1837 |
| 3F1-151T | 151 top | CGTATTGTTCGATCAAGAGNNKCAAGTCGCTAAGGAGGAGAG | 1838 |
| 3F1-151B | 151B | CTCTCCTCCTTAGCGACTTGMNNCTCTTGATCGAACAATACG | 1839 |
| 3F1-174T | 174 top | GCCACTGCTTCGACAAGCTNNKGAACTCAAAGATCACCAAG | 1840 |
| 3F1-174B | 174 bottom | CTTGGTGATCTTTGAGTTCMNNAGCTTGTCGAAGCAGTGGC | 1841 |
| 3F1-177T | 177 top | TCGACAAGCTTGGGAACTCNNKGATCACCAAGGTGCAGAGC | 1842 |
| 3F1-177B | 177 bottom | GCTCTGCACCTTGGTGATCMNNGAGTTCCCAAGCTTGTCGA | 1843 |

Mutagenesis reactions were transformed into library strain Km3 and 96 colonies tested for substitution by DNA sequence analysis. Substitutions representing each possible residue at each position were then re-arrayed in triplicate onto M9 X-gal assay plates with 0, 20 and 200 ppb Chlorsulfuron. Plates were incubated at 37° C. for 24 and 48 hrs prior to imaging. Residue substitutions were then ranked by activation (emphasis on 20 ppb Cs) and repression characteristics (emphasis on 48 hr time point). The mutation with the greatest impact on activity was substitution of residue N82 to phenylalanine or tyrosine. Tryptophan substitution also improved activity at N82 but not nearly as much as either phe or tyr. Substitutions S135D, S135E, F147Q, F147V and S151Q all dramatically increase sensitivity to Chlorsulfuron induction however partially at the expense of repressor function. All other preferred substitutions shown in Table 18 either improved repression or improved sensitivity to inducer without compromising repressor function. Certain residues were indispensible to function such as R104, W105, and W174 as substitutions were not allowed. Other residue positions such as R138 and K177 were also flagged as critical since functional substitutions were extremely limited.

TABLE 18
Summary of saturation mutagenesis results.

| | Residue targeted for mutagenesis | | | | | | | | | | | | | | | | |
|---|---|---|---|---|---|---|---|---|---|---|---|---|---|---|---|---|---|
| | M60 | Q64 | N82 | C86 | C100 | R104 | W105 | A113 | M116 | V134 | S135 | R138 | V139 | F147 | S151 | W174 | K177 |
| Top | H | D | [symbol] | G | A | [symbol] * | [symbol] * | A | L | L | D | H | I | F | G | [symbol] * | [symbol] |
| Substitutions | M | E | [symbol] | M | C | | | G | M | T | E | [symbol] | L | L | Q | | R |
| | N | G | | S | S | | | V | Q | V | G | | V | M | S | | |
| | | Q | | T | | | | | | | S | | | Q | | | |
| | | S | | | | | | | | | | | | V | | | |

Bold = highly sensitive response but slightly leaky;
Bold and italic = highly selected residues;
* = only residue that functions at the respective position

Library CsL3 construction and screening: Based on the IVM results the top performing residue substitutions were incorporated into library CsL3 (Table 14). The library was assembled with the oligonucleotides shown below in Table 19. The first and last primers in each set were used as rescue primers. To enable purification of hit proteins, a 6×His-tag between was added to the C-terminus of the ligand binding domain of each clone during the assembly and rescue process. The library was then inserted into pVER7334 SacI/AscI, transformed into E. coli assay strain Km3 and selected on LB+40 ug/ml Kanamycin and 50 ug/ml Carbenicillin. Approximately 10,000 colonies were then re-arrayed into 384-well format, and replica plated onto M9 XgaI assay medium containing 0 or 20 ppb Cs. Colony color was then assessed at 24 and 96 hrs of incubation at 37° C. Results showed that residue substitutions N82F, V134T, and F147Q were highly preferred as was the maintenance of residues Q64, A113, M116, S135, R138, and V139. Interestingly the very best hits had a random F147L substitution resulting in an additional ˜2× increase in activity over the next best clones. Also, while the C86M substitution was less frequent in the overall hit population it occurred in all top 26 clones.

TABLE 19
Oligonucleotides encoding library CsL3.

| | SEQ ID | | |
|---|---|---|---|
| Oligo | NO | Sequence | Group |
| CsL3:1 | 1844 | TGGCACGTCAAGAACAAGCGAGCTCTGCTAGACGCCTTGGCC | 1 |
| CsL3:2 | 1845 | ATTGAGATGMATGATAGGCACRGCACCCACTACTTGCCTTTG | 2 |
| CsL3:3 | 1846 | ATTGAGATGMATGATAGGCACCAGACCCACTACTTGCCTTTG | |
| CsL3:4 | 1847 | ATTGAGATGATGGATAGGCACRGCACCCACTACTTGCCTTTG | |
| CsL3:5 | 1848 | ATTGAGATGATGGATAGGCACCAGACCCACTACTTGCCTTTG | |
| CsL3:6 | 1849 | GAAGGGGAAAGCTGGCAAGACTTCTTGAGGAACTWCGCTAAG | 3 |
| CsL3:7 | 1850 | AGCTCCCGACGTGCTTTGCTCAGTCACCGTGATGGAGCCAAG | 4 |
| CsL3:8 | 1851 | AGCATGCGACGTGCTTTGCTCAGTCACCGTGATGGAGCCAAG | |
| CsL3:9 | 1852 | GTCKCGCTTGGTACACGGTGGACGGAGCAACAGTATGAAACT | 5 |
| CsL3:10 | 1853 | GSAGAGAACWTGTTGGCCTTCCTGACCCAACAAGGTTTCTCC | 6 |
| CsL3:11 | 1854 | CTTGAGAATGCCTTGTACGCAACCGRCGCTGTGCRTRTTTTC | 7 |
| CsL3:12 | 1855 | CTTGAGAATGCCTTGTACGCAACCTCAGCTGTGCRTRTTTTC | |
| CsL3:13 | 1856 | CTTGAGAATGCCTTGTACGCASTGGRCGCTGTGCRTRTTTTC | |
| CsL3:14 | 1857 | CTTGAGAATGCCTTGTACGCASTGTCAGCTGTGCRTRTTTTC | |
| CsL3:15 | 1858 | ACTCTGGGTGCCGTATTGGTGGATCAAGAGRGCCAAGTCGCT | 8 |
| CsL3:16 | 1859 | ACTCTGGGTGCCGTATTGGTGGATCAAGAGCAGCAAGTCGCT | |
| CsL3:17 | 1860 | ACTCTGGGTGCCGTATTGCAAGATCAAGAGRGCCAAGTCGCT | |
| CsL3:18 | 1861 | ACTCTGGGTGCCGTATTGCAAGATCAAGAGCAGCAAGTCGCT | |
| CsL3:19 | 1862 | AAGGAGGAGAGGGAAACACCTACTACTGATAGTATGCCGCCA | 9 |
| CsL3:20 | 1863 | CTGCTTCGACAAGCCTGGGAACTCAAAGATCACCAAGGTGCA | 10 |
| CsL3:21 | 1864 | GAGCCAGCCTTCCTGTTCGGCCTTGAATTGATCATAGCCGGA | 11 |
| CsL3:22 | 1865 | TTGGAGAAGCAGCTGAAGAGAGAAAGTGGGTCTCACCATCAC | 12 |
| CsL3:23 | 1866 | GTGCCTATCATKCATCTCAATGGCCAAGGCGTCTAGCAGAGC | 13 |
| CsL3:24 | 1867 | GTGCCTATCCATCATCTCAATGGCCAAGGCGTCTAGCAGAGC | |
| CsL3:25 | 1868 | GTCTTGCCAGCTTTCCCCTTCCAAAGGCAAGTAGTGGGTGCT | 14 |
| CsL3:26 | 1869 | GTCTTGCCAGCTTTCCCCTTCCAAAGGCAAGTAGTGGGTGCC | |
| CsL3:27 | 1870 | GTCTTGCCAGCTTTCCCCTTCCAAAGGCAAGTAGTGGGTCTG | |
| CsL3:28 | 1871 | GAGCAAAGCACGTCGGGAGCTCTTAGCGWAGTTCCTCAAGAA | 15 |
| CsL3:29 | 1872 | GAGCAAAGCACGTCGCATGCTCTTAGCGWAGTTCCTCAAGAA | |
| CsL3:30 | 1873 | CCACCGTGTACCAAGCGMGACCTTGGCTCCATCACGGTGACT | 16 |
| CsL3:31 | 1874 | GAAGGCCAACAWGTTCTCTSCAGTTTCATACTGTTGCTCCGT | 17 |
| CsL3:32 | 1875 | TGCGTACAAGGCATTCTCAAGGGAGAAACCTTGTTGGGTCAG | 18 |
| CsL3:33 | 1876 | CACCAATACGGCACCCAGAGTGAAAAYAYGCACAGCGYCGGT | 19 |
| CsL3:34 | 1877 | TTGCAATACGGCACCCAGAGTGAAAAYAYGCACAGCGYCGGT | |
| CsL3:35 | 1878 | CACCAATACGGCACCCAGAGTGAAAAYAYGCACAGCTGAGGT | |
| CsL3:36 | 1879 | TTGCAATACGGCACCCAGAGTGAAAAYAYGCACAGCTGAGGT | |
| CsL3:37 | 1880 | CACCAATACGGCACCCAGAGTGAAAAYAYGCACAGCGYCCAC | |
| CsL3:38 | 1881 | CACCAATACGGCACCCAGAGTGAAAAYAYGCACAGCGYCCAG | |
| CsL3:39 | 1882 | TTGCAATACGGCACCCAGAGTGAAAAYAYGCACAGCGYCCAC | |
| CsL3:40 | 1883 | TTGCAATACGGCACCCAGAGTGAAAAYAYGCACAGCGYCCAG | |
| CsL3:41 | 1884 | CACCAATACGGCACCCAGAGTGAAAAYAYGCACAGCTGACAC | |
| CsL3:42 | 1885 | CACCAATACGGCACCCAGAGTGAAAAYAYGCACAGCTGACAG | |
| CsL3:43 | 1886 | TTGCAATACGGCACCCAGAGTGAAAAYAYGCACAGCTGACAC | |
| CsL3:44 | 1887 | TTGCAATACGGCACCCAGAGTGAAAAYAYGCACAGCTGACAG | |
| CsL3:45 | 1888 | AGGTGTTTCCCTCTCCTCCTTAGCGACTTGGCYCTCTTGATC | 20 |
| CsL3:46 | 1889 | AGGTGTTTCCCTCTCCTCCTTAGCGACTTGCTGCTCTTGATC | |
| CsL3:47 | 1890 | TTCCCAGGCTTGTCGAAGCAGTGGCGGCATACTATCAGTAGT | 21 |
| CsL3:48 | 1891 | GCCGAACAGGAAGGCTGGCTCTGCACCTTGGTGATCTTTGAG | 22 |
| CsL3:49 | 1892 | TCTCTTCAGCTGCTTCTCCAATCCGGCTATGATCAATTCAAG | 23 |
| CsL3:50 | 1893 | CACAGGCGCGCCTTAGTGATGGTGGTGATGGTGAGACCCACTTTC | 24 |

TABLE 20
Performance of the top 20CsL3 hits and associated residue substitutions
relative to the parent clone L8-F301.

| | Colony Assay | | | | | | | | | | | | | | | | | | | | | | | | |
|---|---|---|---|---|---|---|---|---|---|---|---|---|---|---|---|---|---|---|---|---|---|---|---|---|---|
| | Results | | | Residue and Sequence Position | | | | | | | | | | | | | | | | | | | | | |
| CsL3 Hit | REP | IND | F.IND | 60 | 64 | 82 | 86 | 100 | 113 | 121 | 126 | 128 | 134 | 135 | 139 | 147 | 151 | 152 | 155 | 156 | 157 | 158 | 163 | 192 | 202 |
| L8-F301 | 0.7 | 1.6 | 2.5 | M | Q | N | C | C | A | T | S | E | V | S | V | F | S | Q | K | E | E | R | T | L | K |
| 1C12 | 0.9 | 8.8 | 9.5 | H | G | F | M | S | — | — | — | — | T | — | — | L | Q | — | — | — | — | — | — | — | * |
| 1B11 | 1.3 | 10.8 | 8.0 | — | — | F | M | A | — | — | — | — | T | — | — | L | Q | — | — | — | — | — | — | — | — |
| 1A07 | 1.5 | 8.1 | 5.4 | — | — | F | M | S | — | — | P | — | T | D | — | Q | G | — | — | — | — | — | — | — | — |
| 1B04 | 2.2 | 10.5 | 4.8 | H | — | F | M | S | — | — | — | — | T | — | — | Q | Q | — | — | — | — | — | P | — | — |
| 2E09 | 1.3 | 5.7 | 4.5 | H | — | F | M | A | G | — | — | — | T | — | — | Q | G | — | — | — | — | — | — | — | — |
| 2D11 | 0.9 | 3.9 | 4.3 | N | — | F | M | S | — | — | — | — | T | — | — | Q | — | — | — | — | — | — | — | — | — |
| 2B09 | 0.9 | 3.8 | 4.3 | — | — | Y | M | S | — | — | — | — | T | — | — | Q | G | — | N | — | — | — | — | — | — |
| 2B06 | 1.3 | 5.6 | 4.2 | H | — | F | M | S | G | — | — | — | — | D | — | Q | G | — | — | — | D | — | — | — | — |
| 2A01 | 1.4 | 5.9 | 4.2 | — | G | F | M | S | — | — | — | — | T | — | — | Q | — | — | — | — | — | — | — | — | — |
| 2D10 | 1.1 | 4.7 | 4.2 | H | — | F | M | S | — | — | — | — | T | — | — | Q | — | — | — | — | — | — | — | — | — |
| 2D02 | 1.6 | 6.3 | 3.9 | — | — | F | M | S | — | — | P | — | T | D | — | Q | G | — | — | — | — | — | — | — | — |
| 2E07 | 0.9 | 3.4 | 3.8 | — | — | Y | M | A | — | — | — | — | T | — | — | Q | G | — | — | — | — | — | — | — | — |
| 2E12 | 1.2 | 4.4 | 3.8 | — | — | Y | M | A | — | — | — | — | T | G | — | Q | — | H | — | V | — | — | — | — | — |
| 1C01 | 1.5 | 5.5 | 3.7 | — | G | Y | M | A | — | — | — | — | T | G | I | Q | — | — | — | — | — | — | — | V | — |
| 1B05 | 1.3 | 4.8 | 3.6 | — | — | Y | M | A | — | — | — | — | T | — | — | Q | G | — | — | — | — | — | — | — | — |
| 2E10 | 0.4 | 1.3 | 3.5 | H | R | Y | M | S | — | — | — | — | T | — | — | V | Q | — | — | — | — | T | — | — | — |
| 2B12 | 1.7 | 6.1 | 3.5 | — | — | F | M | S | — | — | — | — | T | — | — | Q | — | — | — | — | — | — | — | — | N |
| 2E08 | 2.2 | 7.6 | 3.4 | — | — | F | M | A | — | — | — | — | T | — | — | Q | G | — | — | — | — | — | — | — | — |
| 2E11 | 2.1 | 7.2 | 3.4 | — | — | F | M | S | — | I | — | Q | L | — | — | Q | — | — | — | — | — | — | — | — | — |
| 2D12 | 2.1 | 7.0 | 3.4 | — | S | F | M | A | G | — | — | — | T | — | — | Q | — | — | — | — | — | — | — | — | — |

IND = induction with 20 ppb CS; REP = repression in absence of inducer; F. IND = fold induction (IND/REP)

### D. Sixth Round Chlorsulfuron Repressor Shuffling

Creating novel diversity through random mutagenesis. In order to create new diversity for shuffling the top clone from CsL3 was subjected to error prone PCR mutagenesis using Mutazyme (Stratagene). The mutated PCR product encoding the CsR ligand binding domain was inserted into library expression vector pVER7334 as a SacI to AscI fragment, transformed into library strain Km3 and plated onto LB+40 ug/ml Kanamycin and 50 ug/ml Carbenecillin. Approximately 10,000 colonies were then replica plated onto M9 XgaI assay medium+/−20 ppb Cs. Putative hits were then re-arrayed and replica plated onto the same assay medium. Performance was gauged by the level of blue colony color after 24 hrs incubation on inducer (induction) and 72 hrs incubation without inducer (repression). The top hits were then subjected to liquid B-galactosidase assays for quantitative assessment (Table 21). The results reveal that modification of position D178 is important as mutation to either V or E improves activity at least two-fold. Substitutions F78Y, R88C, and S165R may also have made contributions to activity.

TABLE 21
Performance of the top CsL3-MTZ hits and associated residue substitutions
relative to the parent clone CsL3-C12 and L8-F301.

| | B-galactosidase assay | | | Residue and Sequence Position | | | | | | | | | | | | |
|---|---|---|---|---|---|---|---|---|---|---|---|---|---|---|---|---|
| Clone | IND | REP | F. IND | 60 | 64 | 78 | 82 | 86 | 88 | 100 | 134 | 147 | 151 | 165 | 178 | 202 |
| L8-3F01 | 8 | 7 | 1 | M | Q | F | N | C | R | C | V | F | S | S | D | K |
| CsL3-C12 | 218 | 17 | 13 | H | G | — | F | M | — | S | T | L | Q | — | — | * |
| CsL3-C12-MTZ-2 | 287 | 9 | 30 | H | G | — | F | M | — | S | T | L | Q | — | V | * |
| CsL3-C12-MTZ-4 | 460 | 18 | 25 | H | G | Y | F | M | — | S | T | L | Q | — | E | * |
| CsL3-C12-MTZ-3 | 347 | 21 | 16 | H | G | — | F | M | C | S | T | L | Q | R | — | * |
| CsL3-C12-MTZ-5 | 440 | 29 | 15 | H | G | — | F | M | — | S | T | L | Q | — | E | * |

IND = induction with 20 ppb CS; REP = repression in absence of inducer; F. IND = fold induction (IND/REP)

Construction and screening of library CsL4.2. Seventh round library CsL4.2 was designed based on the best diversity from CsL3 and CsL3-MTZ library screens (Table 14). The library was assembled with oligonucleotides shown below in Table 22. The first and last primers were used as rescue primers. CsL4.2 included a C-terminal 6×His-tag extension to facilitate protein purification. The library was assembled and cloned into vector pVER7334 SacI to AscI, transformed into library assay strain Km3 and plated onto LB+40 ug/ml Kanamycin and 50 ug/ml carbenecillin. Approximately 8,000 colonies were re-arrayed into 384-well format and replica plated onto M9 XgaI assay medium+/−2 ppb Cs. Putative hits were re-arrayed in 96-well format onto the same media for re-testing. Confirmed hits were then tested for induction and repression aspects in liquid culture using B-galactosidase assays. Results show that F82, L147, V178, and to a lesser extent Q151 were strongly selected for in the hit population. Although there was no preference at position 135 in the larger hit population, the top six clones all had the S135D substitution (Table 23).

TABLE 22
Library 4.2 assembly oligonucleotides.

| | SEQ ID | | |
|---|---|---|---|
| Oligo | NO | Sequence | Group |
| CsL4.2-1 | 1894 | TGGCACGTCAAGAACAAGCGAGCTCTGCTAGACGCCTTGGCC | 1 |
| CsL4.2-2 | 1895 | ATTGAGATGCATGATAGGCACGGAACCCACTACTTGCCTTTG | 2 |
| CsL4.2-3 | 1896 | ATTGAGATGCATGATAGGCACCAAACCCACTACTTGCCTTTG | |
| CsL4.2-4 | 1897 | ATTGAGATGATGGATAGGCACGGAACCCACTACTTGCCTTTG | |
| CsL4.2-5 | 1898 | ATTGAGATGATGGATAGGCACCAAACCCACTACTTGCCTTTG | |
| CsL4.2-6 | 1899 | GAAGGGGAAAGCTGGCAAGACTWTTTGAGGAACTWTGCTAAG | 3 |
| CsL4.2-7 | 1900 | AGCATGCGACKAGCTTTGCTCAGTCACCGTGATGGAGCCAAG | 4 |
| CsL4.2-8 | 1901 | AGCATGCGATGCGCTTTGCTCAGTCACCGTGATGGAGCCAAG | |
| CsL4.2-9 | 1902 | GTCKCCCTTGGTACACGGTGGACGGAGCAACAGTATGAAACT | 5 |
| CsL4.2-10 | 1903 | GCGGAGAACATGTTGGCCTTCCTGACCCAACAAGGTTTCTCC | 6 |
| CsL4.2-11 | 1904 | CTTGAGAATGCCTTGTACGCAACAGATGCTGTGCGGGTTTTC | 7 |
| CsL4.2-12 | 1905 | CTTGAGAATGCCTTGTACGCAACAAGCGCTGTGCGGGTTTTC | |
| CsL4.2-13 | 1906 | ACTCTGGGTGCCGTATTGCWGGATCAAGAGGGACAAGTCGCT | 8 |
| CsL4.2-14 | 1907 | ACTCTGGGTGCCGTATTGCWGGATCAAGAGCAACAAGTCGCT | |
| CsL4.2-15 | 1908 | AAKGAGGAGAGGGAAACACCTACTMCTGATAGWATGCCGCCA | 9 |
| CsL4.2-16 | 1909 | CTGCTTCGACAAGCCTGGGAACTCAAAGWKCACCAAGGTGCA | 10 |
| CsL4.2-17 | 1910 | GAGCCAGCCTTCCTGTTCGGCCTTGAATTGATCATAGCCGGA | 11 |
| CsL4.2-18 | 1911 | TTGGAGAAGCAGCTGAAGAGAGAAAGTGGGTCTCACCATCAC | 12 |
| CsL4.2-19 | 1912 | GTGCCTATCATGCATCTCAATGGCCAAGGCGTCTAGCAGAGC | 13 |
| CsL4.2-20 | 1913 | GTGCCTATCCATCATCTCAATGGCCAAGGCGTCTAGCAGAGC | |
| CsL4.2-21 | 1914 | GTCTTGCCAGCTTTCCCCTTCCAAAGGCAAGTAGTGGGTTCC | 14 |
| CsL4.2-22 | 1915 | GTCTTGCCAGCTTTCCCCTTCCAAAGGCAAGTAGTGGGTTTG | |
| CsL4.2-23 | 1916 | GAGCAAAGCTMGTCGCATGCTCTTAGCAWAGTTCCTCAAAWA | 15 |
| CsL4.2-24 | 1917 | GAGCAAAGCGCATCGCATGCTCTTAGCAWAGTTCCTCAAAWA | |
| CsL4.2-25 | 1918 | CCACCGTGTACCAAGGGMGACCTTGGCTCCATCACGGTGACT | 16 |
| CsL4.2-26 | 1919 | GAAGGCCAACATGTTCTCCGCAGTTTCATACTGTTGCTCCGT | 17 |
| CsL4.2-27 | 1920 | TGCGTACAAGGCATTCTCAAGGGAGAAACCTTGTTGGGTCAG | 18 |
| CsL4.2-28 | 1921 | CWGCAATACGGCACCCAGAGTGAAAACCCGCACAGCATCTGT | 19 |
| CsL4.2-29 | 1922 | CWGCAATACGGCACCCAGAGTGAAAACCCGCACAGCGCTTGT | |
| CsL4.2-30 | 1923 | AGGTGTTTCCCTCTCCTCMTTAGCGACTTGTCCCTCTTGATC | 20 |
| CsL4.2-31 | 1924 | AGGTGTTTCCCTCTCCTCMTTAGCGACTTGTTGCTCTTGATC | |
| CsL4.2-32 | 1925 | TTCCCAGGCTTGTCGAAGCAGTGGCGGCATWCTATCAGKAGT | 21 |
| CsL4.2-33 | 1926 | GCCGAACAGGAAGGCTGGCTCTGCACCTTGGTGMWCTTTGAG | 22 |
| CsL4.2-34 | 1927 | TCTCTTCAGCTGCTTCTCCAATCCGGCTATGATCAATTCAAG | 23 |
| CsL4.2-35 | 1928 | CACAGGCGCGCCTTAGTGATGGTGGTGATGGTGAGACCCACTTTC | 24 |

TABLE 23
Performance of the top 20 CsL4.2 hits and associated residue substitutions
relative to the parent clone L8-F301.

| | B- | | | | | | | | | | | | | | | | | | | | | | | | | | |
|---|---|---|---|---|---|---|---|---|---|---|---|---|---|---|---|---|---|---|---|---|---|---|---|---|---|---|---|
| | galactosidase | | | Residue and Sequence Position | | | | | | | | | | | | | | | | | | | | | | | |
| Clone | REP | IND | F. IND | 60 | 64 | 78 | 82 | 86 | 88 | 99 | 100 | 119 | 123 | 134 | 135 | 147 | 151 | 155 | 156 | 157 | 163 | 165 | 171 | 178 | 193 | 202 | 204 |
| L8-3F01 | 0.4 | 0.9 | 2.0 | M | Q | F | N | C | R | V | C | F | Q | V | S | F | S | K | E | E | T | S | R | D | I | K | E |
| CsL4.2-20 | 0.2 | 7.4 | 39.8 | H | — | — | F | M | L | — | S | — | — | T | D | L | Q | — | — | — | P | — | — | V | — | — | — |
| CsL4.2-15 | 0.2 | 4.0 | 25.5 | H | — | — | F | M | — | — | S | — | — | T | D | L | Q | — | — | — | — | — | — | V | — | — | — |
| CsL4.2-22 | 0.3 | 5.4 | 20.8 | H | — | — | F | M | — | I | A | — | — | T | D | L | Q | — | — | — | P | — | — | — | — | — | — |
| CsL4.2-07 | 0.3 | 6.5 | 18.9 | H | — | Y | F | M | C | I | A | — | — | T | D | L | Q | — | — | — | P | — | — | V | — | — | — |
| CsL4.2-16 | 0.3 | 3.8 | 15.2 | — | — | Y | F | M | C | — | S | — | — | T | D | L | G | N | — | — | P | R | — | V | — | — | — |
| CsL4.2-08 | 0.7 | 10.7 | 15.0 | — | — | — | F | M | — | — | A | — | H | T | D | L | Q | — | — | — | P | — | — | V | — | — | — |
| CsL4.2-24 | 0.4 | 5.4 | 14.3 | H | — | Y | F | M | C | — | A | — | — | T | — | L | G | N | — | — | P | — | — | V | — | — | — |
| CsL4.2-21 | 0.2 | 3.2 | 13.2 | — | G | — | Y | M | — | — | A | C | — | T | — | L | Q | N | — | — | — | — | — | V | — | — | — |
| CsL4.2-28 | 0.5 | 5.3 | 11.3 | — | — | Y | F | M | C | — | A | — | — | T | — | L | Q | — | Q | — | P | — | — | V | — | — | — |
| CsL4.2-30 | 0.5 | 4.9 | 10.8 | H | — | — | F | M | — | — | A | — | — | T | — | L | G | N | — | — | P | — | — | V | — | — | — |
| CsL4.2-26 | 0.3 | 3.1 | 10.6 | H | — | Y | F | M | C | — | S | — | — | T | — | L | Q | — | — | — | P | R | — | V | — | — | — |
| CsL4.2-23 | 1.0 | 10.4 | 10.5 | — | — | Y | F | M | C | — | A | — | — | T | — | L | Q | — | — | — | P | R | — | V | — | — | — |
| CsL4.2-04 | 0.4 | 4.3 | 10.2 | H | — | — | F | M | C | — | A | — | — | T | D | L | Q | N | — | G | — | — | — | E | — | — | — |
| CsL4.2-01 | 0.4 | 3.8 | 9.8 | H | — | Y | F | M | — | — | A | — | — | T | D | L | G | — | — | — | — | — | — | V | — | — | — |
| CsL4.2-17 | 0.3 | 3.1 | 9.7 | — | — | Y | F | M | C | — | A | — | — | T | — | L | Q | — | — | — | — | — | — | V | — | — | — |
| CsL4.2-12 | 0.7 | 6.4 | 9.5 | H | G | — | F | M | — | — | A | — | — | T | — | L | G | N | — | — | — | — | — | V | — | — | — |
| CsL4.2-18 | 0.7 | 6.8 | 9.3 | — | — | — | F | M | C | — | A | — | — | T | — | L | Q | — | — | — | P | R | — | V | L | — | — |
| CsL4.2-27 | 0.4 | 3.2 | 9.1 | — | — | — | F | M | C | — | S | — | — | T | D | L | Q | — | — | — | P | R | Q | E | — | — | D |
| CsL4.2-11 | 0.5 | 4.8 | 8.9 | H | G | Y | F | M | — | — | S | — | — | T | — | L | Q | — | — | — | — | — | — | E | — | X | — |

IND = induction with 20 ppb CS; REP = repression in absence of inducer; F. IND = fold induction (IND/REP)

### E. In Vitro Mutagenesis of Residue D178

Since residue position D178 [relative to TetR(B)] was found by random mutagenesis to be important for activity further mining was sought. To this end, saturation mutagenesis was performed at this position on top CsR hits CsL4.2-15 and CsL4.2-20 using the following top and bottom strand primers in a Phusion DNA polymerase PCR reaction (New England Biolabs):

(SEQ ID NO: 2136)
GCCTGGGAACTCAAANNKCACCAAGGTGCAGAGC
and
(SEQ ID NO: 2137)
GCTCTGCACCTTGGTGMNNTTTGAGTTCCCAGGC.

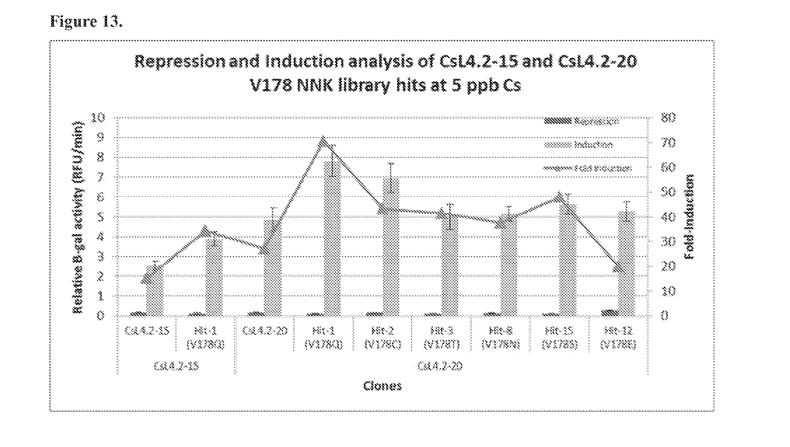

Mutagenesis reactions were transformed into E. coli assay strain Km3 and plated onto LB+50 ug/ml Carbenecillin. Colonies were then re-arrayed into 384 well format and replica plated onto M9 XgaI assay medium+/−5 ppb Chlorsulfuron. Putative hits were then re-arrayed and analyzed by B-galactosidase assays relative to the parent clones (FIG. 13). The results show that V178 substitutions in CsL4.2-20 to C, N, Q, S, or T all yield improved activity. However, the most active substitution, V178Q, led to an approximately 2× improvement in both CsL4.2-15 and CsL4.2-20 backbones.

### F. Modification of SU Selectivity Thru Binding Pocket Mutagenesis

### Example 5

### Crystal Structure Determination of CsR(CsL4.2)

To better understand the mechanism of the engineered sulfonylurea repressors and to help guide future design/selection efforts, the crystal structures of two repressor variants were solved by x-ray crystallography in the presence and absence of their respective ligands. The structures of ethametsulfuron repressors EsR(L7-D1 also referred to as L7-3E03 in table 1B) and EsR(L11-C6 also referred of as L11-17(C06) in table 1B) were determined in their ligand-free and ethametsulfuron-bound states, respectively. The chlorsulfuron repressor variant CsR(L4.2-20) was solved both with and without chlorsulfuron bound. The atomic coordinates from these crystal structures were determined and deposited at Protein Data Bank (PDB).

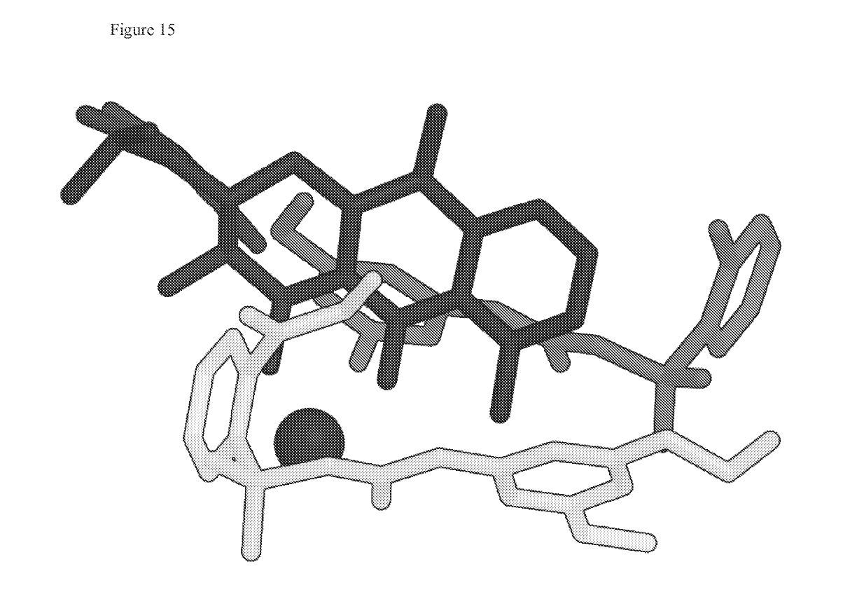

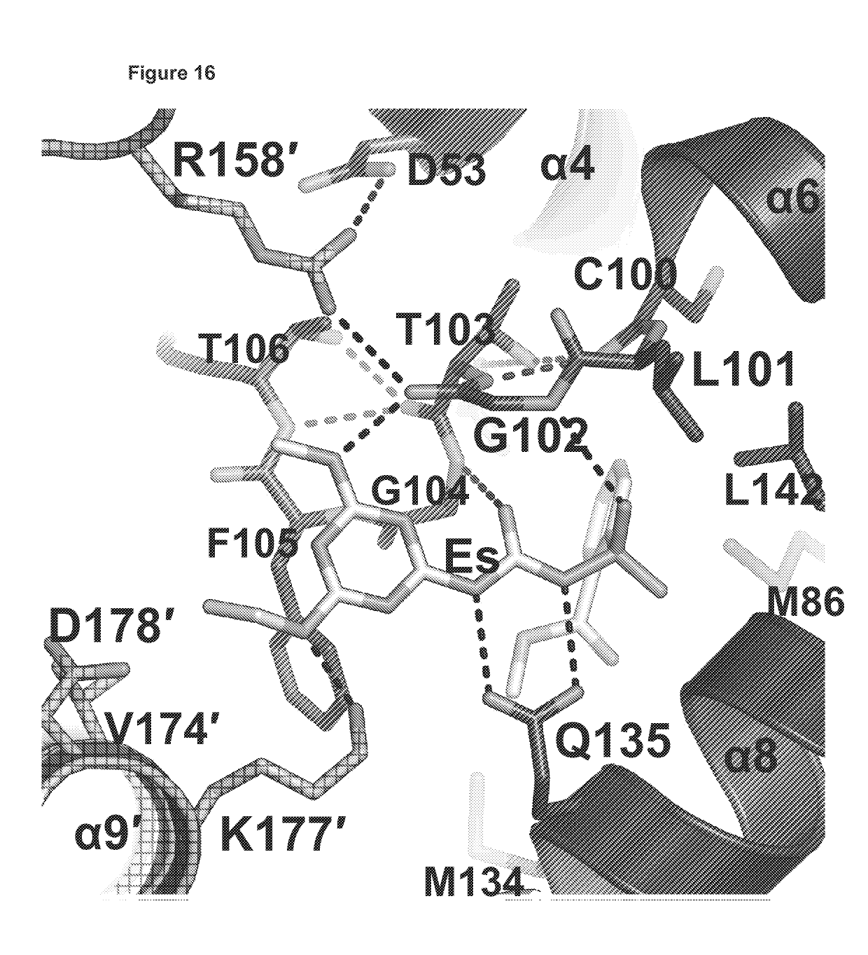

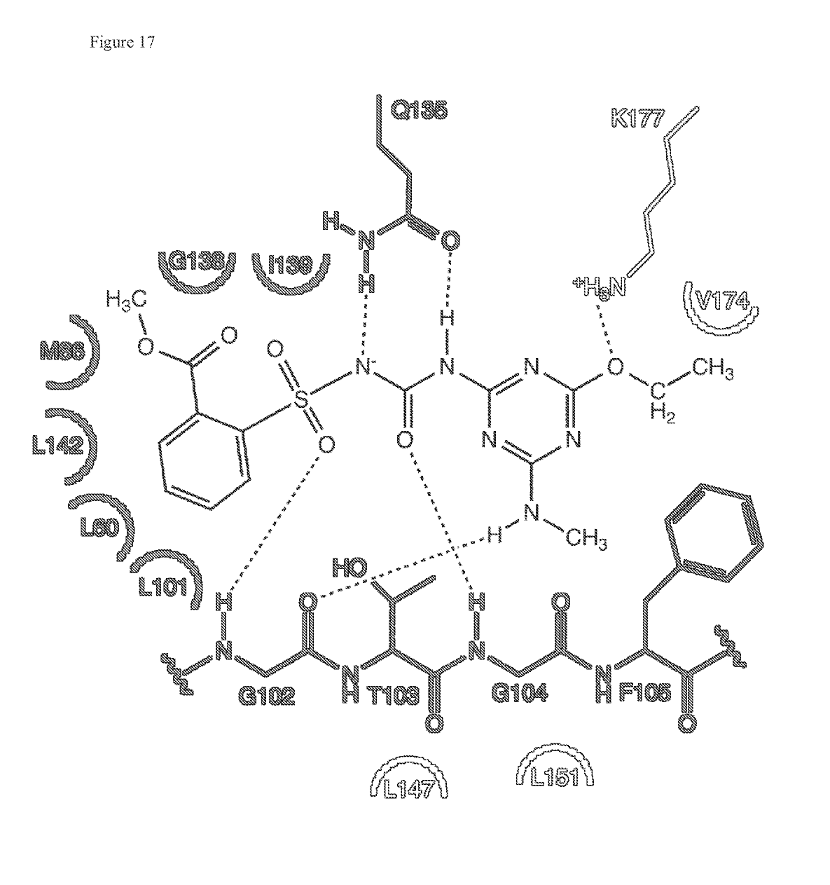

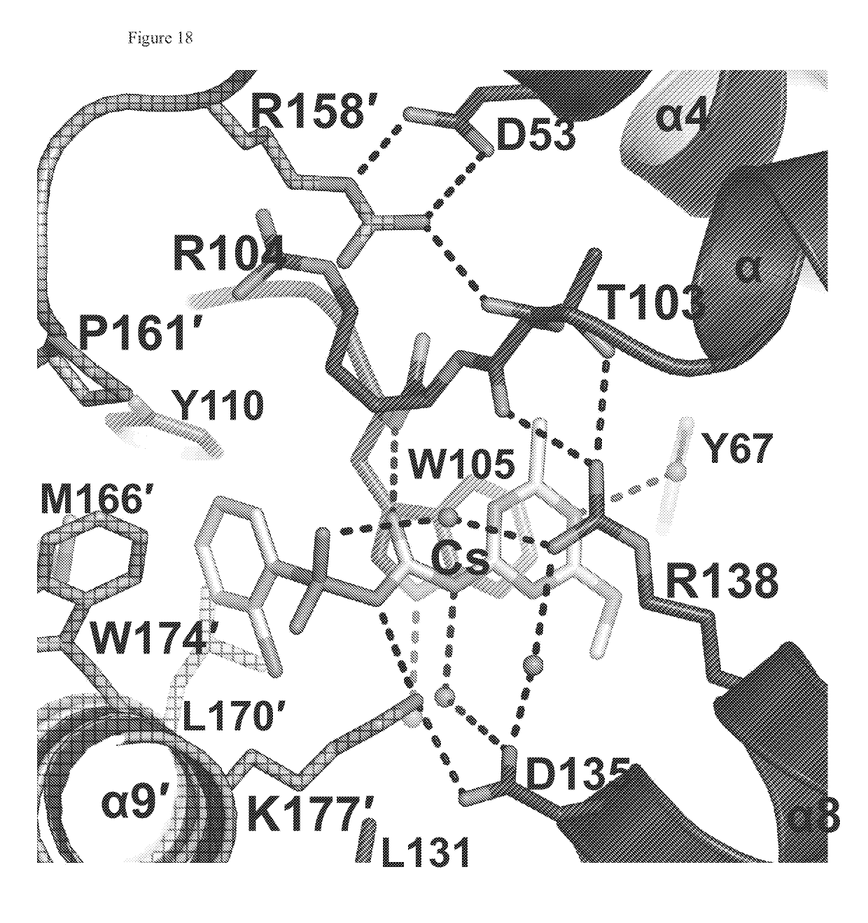

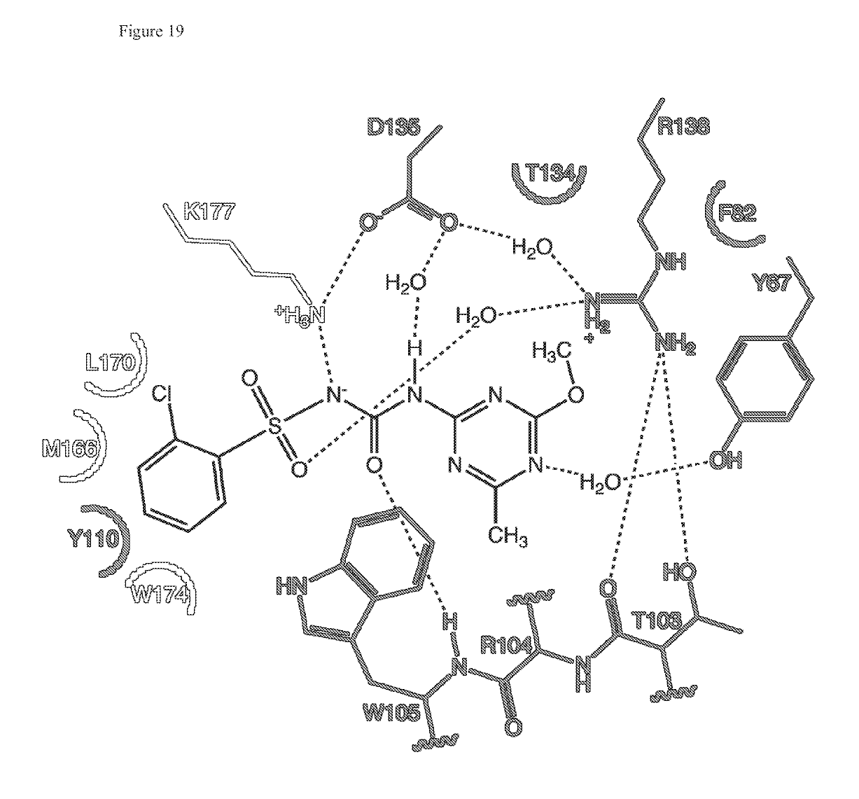

All structures showed a dimeric organization for the repressors, with helical structures generally similar to the tetracycline repressor, both in the ligand-bound and ligand-free states. In ligand-bound structures, the ligands Es and Cs were observed bound to the equivalent binding pockets where tetracycline binds to TetR. However, the orientation of the ligands and mode of interaction with the respective repressor were distinct from each other and from tetracycline (FIG. 15). Numerous specific polar and non-polar interactions were observed between the sulfonylurea repressors and their bound ligands (FIGS. 16-19).

The determination of the high-resolution crystal structures, particularly those in complex with the target ligands, has dramatically improved the ability to target the proteins for systematic improvement. Most importantly, the structures have allowed delineation of the positions of the repressors into three classes: 1) those absolutely critical for target ligand binding with no possibility of mutation, e.g. side-chains making “lynchpin” interactions with the SU backbone, 2) those that are somewhat flexible, such as side-chains making interactions with SU appendages, and 3) those that are effectively uninvolved in SU binding, the resulting conformational change, or DNA binding.

The crystal structures allow targeting research efforts to type #2 positions of the protein. The principal types of improvements that were made from the structures were mutations to improve ligand-binding affinity and selectivity. Most importantly, improvements in affinity allow effective responses at lower concentrations of the inducer, both facilitating greater penetration of induction response into plant tissue with the same dosage, and ideally use of less chemical. The increase in repressor/inducer binding affinity over the many rounds of directed evolution is consistent with type #2 protein positions contributing strongly to binding affinity. Such contributions apparently manifest both as direct interactions with SU and by more indirect relationships, such as positions facilitating ligand-dependent conformational change.

For binding specificity for the target ligand(s), several types of improvements are possible. Primarily, increased specificity for a specific SU ligand over other SUs permits the creation of multiple, orthogonal repressor/SU pairs, such as select EsR and CsR variants, which effectively show no cross-talk between the repressor/inducer pairs, allowing them to be used in conjunction with each other. This permits either independent activation of two transgenes, or independent activation and silencing of a single transgene. A secondary application of selectivity modulation is to engineer the SU repressors to be less specific for single SUs over others, while maintaining the core repressor-sulfonylurea interactions. This would create a repressor that could be modularly used with a broad range of SU herbicides, which is useful as the SU molecules have different tissue-penetration and persistence properties, in the case of different SUs being applied to a given crop. In addition, use of a single repressor between crops would lower regulatory hurdles and streamline workflow of repressor/inducer dissemination.

### Example 6

### Enhancement of Ligand Selectivity Thru Structure Guided Mutagenesis

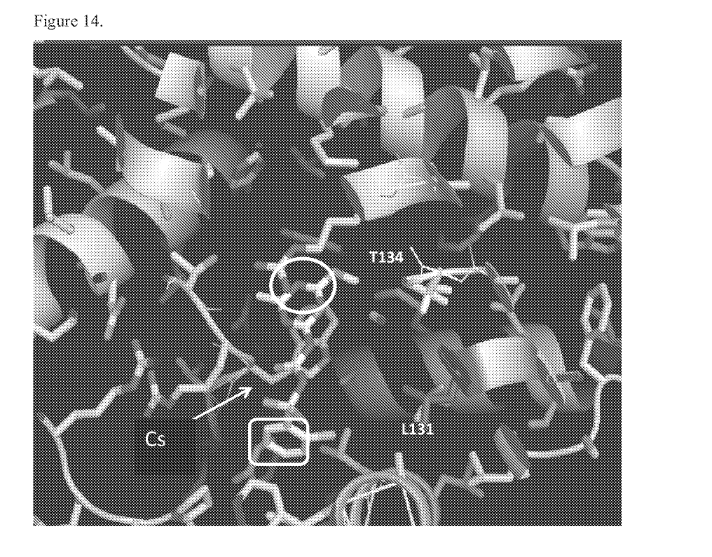

Chlorsulfuron (Cs) repressor CsL4.2-20 is approximately 2- and 30-fold more sensitive to Cs than Metsulfuron (Ms) and Ethametsulfuron (Es), respectively (Table 26). In order to develop non-overlapping SU herbicide responsive repressors it is desired to further separate their ligand spectrum. From the CsL4.2-20 structural model we determined that residues A56, T103, Y110, L117, L131, T134, R138, P161, M166, and A173 could potentially influence docking of related sulfonylurea compounds (e.g. note L131 and T134 in FIG. 14). Cs and Es differ in decoration of both the phenyl and triazine ring structures (circled in FIG. 14). Cs has a chloride (Cl) group in the ortho position on the phenyl ring whereas it is a carboxymethyl group in Es. In addition, the meta-positions of the triazine moiety on both molecules have different substitutions: methyl and methyl-ether on Cs vs secondary amine and ethyl-ether groups on Es. Metsulfuron is essentially a hybrid between these two herbicides in that it has the triazine moiety from Cs and the phenyl moiety from Es. Saturation mutagenesis primers for each residue target are shown below. Mutagenesis reactions were carried out using Phusion DNA polymerase (New England Biolabs) and the primers listed in Table 24 and Table 25. Reactions were transformed into E. coli assay strain Km3 and plated onto LB+50 ug/ml Carbenecillin. Colonies were re-arrayed into 384-well format and replica plated onto M9 X-gal assay medium with no inducer, 10 ppb Es, 200 ppb Es, and 25 ppb Ms. Mutants having shifted selectivity relative to parent Cs activity were re-arrayed into 96-well format for further study. Putative hits were tested for repression and induction with 1, 2.5, 5, and 10 ppb Cs; 25, 50, 100, and 200 ppb Ms; and 200, 250, 300, 350, 400, 450 and 500 ppb Es. The dose of each ligand required to elicit an equal response was then used to determine relative selectivity for each clone. The ratio of Cs to Es and Cs to Ms activities as well as the relative Cs activity for the top hits is presented in Table 25. These data show that positions L131 and T134 were especially useful in modifying ligand selectivity. Mutations L131K and T134W effectively blocked Es activation: 500 ppb Es gave a similar response to 1 ppb Cs. The latter substitution unfortunately reduces Cs activity by ˜2-fold. Other residue substitutions at these positions also impact selectivity to a lesser degree. Interestingly, some mutations increased the response to Cs such as L131C while reducing, but not eliminating, Es activity. Changes in selectivity towards Ms, while occurring in most of the L131 and T134 mutants, were more modest as Cs and Ms are more similar than Cs and Es in structure.

TABLE 24
Oligonucleotides used for saturation mutagenesis of residues
potentially involved in selectivity of different sulfonylurea
herbicides.

| Oligo | Sequence | SEQ ID NO |
|---|---|---|
| A56NNKT | GCTCTGCTAGACGCCTTGNNKATTGAGATGCATGATAGGC | 1929 |
| A56NNKB | GCCTATCATGCATCTCAATMNNCAAGGCGTCTAGCAGAGC | 1930 |
| T103NNKT | GCCAAGGTCTCCCTTGGTNNKCGGTGGACGGAGCAAC | 1931 |
| T103NNKB | GTTGCTCCGTCCACCGMNNACCAAGGGAGACCTTGGC | 1932 |
| Y110NNKT | GGTGGACGGAGCAACAGNNKGAAACTGCGGAGAAC | 1933 |
| Yl10NNKB | GTTCTCCGCAGTTTCMNNCTGTTGCTCCGTCCACC | 1934 |
| L117NNKT | GAAACTGCGGAGAACATGNNKGCCTTCCTGACCCAAC | 1935 |
| L117NNKB | GTTGGGTCAGGAAGGCMNNCATGTTCTCCGCAGTTTC | 1936 |
| L131NNKT | GGTTTCTCCCTTGAGAATGCCNNKTACGCAACAGATGC | 1937 |

TABLE 25
Oligonucleotides used for saturation mutagenesis of residues
potentially involved in selectivity of different sulfonylurea
herbicides.

| Oligo | Sequence | SEQ ID NO |
|---|---|---|
| L131NNKB | GCATCTGTTGCGTAMNNGGCATTCTCAAGGGAGAAACC | 1938 |
| T134NNKT | GAATGCCTTGTACGCANNKGATGCTGTGCGGGTTTTC | 1939 |
| T134NNKB | GAAAACCCGCACAGCATCMNNTGCGTACAAGGCATTC | 1940 |
| R138NNKT | GCAACAGATGCTGTGNNKGTTTTCACTCTGGGTGC | 1941 |
| R138NNKB | GCACCCAGAGTGAAAACMNNCACAGCATCTGTTGC | 1942 |
| P161NNKT | GAGGAGAGGGAAACANNKACTCCTGATAGTATGC | 1943 |
| P161NNKB | GCATACTATCAGGAGTMNNTGTTTCCCTCTCCTC | 1944 |
| M166NNKT | GAAACACCTACTCCTGATAGTNNKCCGCCACTGCTTC | 1945 |
| M166NNKB | GAAGCAGTGGCGGMNNACTATCAGGAGTAGGTGTTTC | 1946 |
| A173NNKT | GCCACTGCTTCGACAANNKTGGGAACTCAAAGTTC | 1947 |
| A173NNKB | GAACTTTGAGTTCCCAMNNTTGTCGAAGCAGTGGC | 1948 |

TABLE 26
Relative Cs, Es, and Ms selectivity of various hits based on
B-galactosidase assays.

| Residue | Relative B-galactosidase activity | | |
|---|---|---|---|
| Substitution | Cs | Cs/Es | Cs/Ms |
| None | 1.0 | 30 | 2.0 |
| L131K | 1.0 | 200 | 20.0 |
| L131H | 1.0 | 80 | 3.3 |
| L131A | 0.5 | 60 | 1.7 |
| L131C | 2.0 | 60 | 4.0 |
| T134S | 0.5 | 40 | 2.5 |
| T134W | 0.5 | 100 | 1.6 |

Relative B-galactosidase activity was determined at various doses of Cs, Es, and Ms.
The amount of each inducer required to achieve the same level of activity was used to determine relative ligand selectivity.

The article “a” and “an” are used herein to refer to one or more than one (i.e., to at least one) of the grammatical object of the article. By way of example, “an element” means one or more element.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

1. A polynucleotide construct comprising a nucleotide sequence encoding a polypeptide having a sulfonylurea (SU)-dependent stabilization domain.

2. The polynucleotide construct of claim 1, wherein said SU-dependent stabilization domain comprises
(a) a ligand binding domain of a SU chemically-regulated transcriptional regulator having at least one destabilization mutation;
(b) a DNA binding domain of a SU chemically-regulated transcriptional regulator having at least one destabilization mutation; or
(c) said SU-dependent stabilization domain comprises both (a) and (b).

3. The polynucleotide construct of claim 1, wherein the ligand binding domain of the SU chemically-regulated transcriptional regulator comprises a polypeptide having at least 80%, 85%, 90%, or 95% sequence identity to the ligand binding domain of an amino acid sequences sequence set forth in any one of SEQ ID NO:3-419, wherein said polypeptide further comprises at least one destabilization mutation.

4. The polynucleotide construct of claim 1, wherein the encoded polypeptide having the SU-dependent stabilization domain comprises a SU chemically-regulated transcriptional regulator.

5. The polynucleotide construct of claim 4, wherein the SU chemically-regulated transcriptional regulator comprise a reverse SU chemically-regulated transcriptional repressor (revSuR).

6. The polynucleotide construct of claim 4, wherein said SuR shares at least 80%, 85%, 90%, or 95% sequence identity to any one of the polypeptides set forth in SEQ ID NO:3-411, wherein said polypeptide further comprises at least one destabilization mutation.

7. The polynucleotide construct of claim 5, wherein said revSuR shares at least 80%, 85%, 90%, or 95% sequence identity to any one of the polypeptides set forth any one of SEQ ID NO:412-419, wherein said polypeptide further comprises at least one destabilization mutation.

8. The polynucleotide construct of claim 5, wherein the revSuR further comprises a transcriptional activator.

9. The polynucleotide construct of claim 2, wherein said destabilization mutation comprises the L17G mutation, the G96R mutation, or any combination thereof.

10. The polynucleotide construct of claim 8, wherein said destabilization mutation comprises the L17G mutation, the G96R mutation, or any combination thereof.

11. The polynucleotide construct of claim 1, wherein said nucleotide sequence encoding the polypeptide having the SU-dependent stabilization domain is operably linked to a polynucleotide encoding a polypeptide of interest.

12. The polynucleotide construct of claim 11, further comprises a nucleotide sequence encoding an intein.

13. The polynucleotide construct of claim 1, wherein said SU comprises a pyrimidinylsulfonylurea, a triazinylsulfonylurea, a thiadazolylurea, a chlorosulfuron, an ethametsulfuron, a thifensulfuron, a metsulfuron, a sulfometuron, a tribenuron, a chlorimuron, a nicosulfuron, or a rimsulfuron compound.

14. A DNA construct comprising the polynucleotide construct of claim 1, wherein said polynucleotide is operably linked to a promoter.

15-17. (canceled)

18. A cell having the recombinant polynucleotide of claim 1 or the DNA construct of claim 15.

19-21. (canceled)

22. A plant comprising the cell of claim 18.

23-24. (canceled)

25. A method to modulate the stability of a polypeptide of interest in a cell comprising:
a) providing a cell having a recombinant polynucleotide comprising a nucleotide sequence encoding a polypeptide having a sulfonylurea (SU)-dependent stabilization domain operably linked to a polynucleotide encoding the polypeptide of interest;
b) expressing the recombinant polynucleotide in the cell; and,
c) contacting the cell with an effective amount of a SU ligand, wherein the effective amount of the SU ligand increases the level the polypeptide of interest in the cell.

26-44. (canceled)

45. A cell comprising
a) a first recombinant construct comprising a first promoter operably linked to a SU chemically-regulated transcriptional regulator comprising a reverse SU repressor (revSuR) comprising a transcriptional activator domain, wherein said revSuR comprises a destabilization mutation; and,
b) a second recombinant construct comprising a first ligand responsive promoter comprising at least one, two or three cognate operators for said SU chemically-regulated transcriptional activator operably linked to a polynucleotide of interest.

46-57. (canceled)

58. A method to regulate expression in a plant, comprising
(a) providing a cell comprising
(i) a first recombinant construct comprising a first promoter operably linked to a SU chemically-regulated transcriptional regulator comprising a reverse SU repressor (revSuR) comprising a transcriptional activator domain, wherein said revSuR comprises a destabilization mutation; and,
(ii) a second recombinant construct comprising a first ligand responsive promoter comprising at least one, two or three cognate operators for said revSuR operably linked to a polynucleotide of interest; and,
(b) providing the cell with an effective amount of the SU ligand whereby the effective amount of the SU ligand increases the level of the revSuR and increases the level of polynucleotide of interest.

59. The method of claim 58, wherein said destabilization mutation is found within
(a) a ligand binding domain of the revSuR;
(b) a DNA binding domain of the revSuR; or
(c) both said ligand binding domain and said DNA binding domain.

60. The method of claim 58, wherein said revSuR shares at least 80%, 85%, 90%, or 95% sequence identity to any one of the polypeptides set forth any one of SEQ ID NO:412-419, wherein said polypeptide further comprises at least one destabilization mutation.

61. The method of claim 58, wherein said destabilization mutation comprises the L17G mutation, the G96R mutation, or any combination thereof.

62-69. (canceled)
